(19) United States
(12) Patent Application Publication
(10) Pub. No.: US 2003/0124579 A1
(43) Pub. Date: Jul. 3, 2003

(54) Methods of diagnosis of ovarian cancer, compositions and methods of screening for modulators of ovarian cancer

(75) Inventors: David H. Mack, Menlo Park, CA (US); Kurt C. Gish, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Eos Biotechnology, Inc., South San Francisco, CA

(21) Appl. No.: 10/235,399

(22) Filed: Sep. 4, 2002

Related U.S. Application Data
(60) Provisional application No. 60/372,246, filed on Apr. 12, 2002.
(60) Provisional application No. 60/350,666, filed on Nov. 13, 2001.
(60) Provisional application No. 60/317,544, filed on Sep. 5, 2001.

(51) Int. Cl. C12Q 1/68; C07H 21/04; C12N 9/00; C12P 21/02; C12N 5/06

(52) U.S. Cl. 435/6; 435/69.1; 435/183; 435/320.1; 435/325; 536/23.1

(57) ABSTRACT

Described herein are genes whose expression are up-regulated or down-regulated in ovarian cancer. Related methods and compositions that can be used for diagnosis and treatment of ovarian cancer are disclosed. Also described herein are methods that can be used to identify modulators of ovarian cancer.

### CROSS-REFERENCES TO RELATED APPLICATIONS

This application claims priority to U.S. S No. 60/317,544 filed Sep. 5, 2001, U.S. S No. 60/350,666 filed Nov. 13, 2001, and U.S. S No. 60/372,246 filed Apr. 12, 2002, each of which is incorporated herein by reference.

### FIELD OF THE INVENTION

The invention relates to the identification of nucleic acid and protein expression profiles and nucleic acids, products, and antibodies thereto that are involved in ovarian cancer; and to the use of such expression profiles and compositions in the diagnosis, prognosis and therapy of ovarian cancer. The invention further relates to methods for identifying and using agents and/or targets that inhibit ovarian cancer.

### BACKGROUND OF THE INVENTION

Ovarian cancer is the sixth most common cancer in women, accounting for 6% of all female cancers. It ranks fifth as the cause of cancer death in women. The American Cancer Society predicts that there will be about 23,100 new cases of ovarian cancer in this country in the year 2000 and about 14,000 women will die of the disease. Because many ovarian cancers cannot be detected early in their development, they account for a disproportionate number of fatal cancers, being responsible for almost half the deaths from cancer of the female genital tract; more deaths than any other reproductive organ cancer.

Most patients with epithelial ovarian cancer, the predominant form, are asymptomatic in early-stage disease and usually present with stage III or IV disease. Their five-year survival is less than 25%, with lower survival among African-American women. The minority of patients discovered with early-stage disease have a five-year survival rate of 80%-90% (Parker, S. L. et. al. Cancer statistics, 1997. CA 1997: 47: 5-27).

In the absence of a family history of ovarian cancer, lifetime risk of ovarian cancer is 1/70. Risk factors include familial cancer syndromes (risk of up to 82% by age 70 in women with hereditary breast/ovarian syndrome); family history (1.4% lifetime risk with no affected relatives, 5% with one affected relative, 7% with two affected relatives; Kerlikowske, K. et.al. Obstet Gynecol (1992) 80: 700-707) nulliparity; advancing age; obesity; personal history of breast, endometrial, or colorectal cancer; fewer pregnancies; or older age (>35 years) at first pregnancy. However, 95% of all ovarian cancers occur in women without risk factors. Use of hormonal contraceptives, oophorectomy, and tubal sterilization reduce risk of ovarian cancer (Kerlikowske, K. et. al. Obstet Gynecol (1992) 80: 700-707; Grimes, D. A. Am J. Obstet. Gynecol. (1992) 166: 1950-1954; Hankinson, S. E. et. al. (1993) JAMA 270: 2813-2818) however, even bilateral oophorectomy may not be completely effective in preventing ovarian cancer.

Treatment of ovarian cancer consists largely of surgical oophectemy, anti-hormone therapy, and/or chemotherapy. Although many ovarian cancer patients are effectively treated, the current therapies can all induce serious side effects which diminish quality of life. Deciding on a particular course of treatment is typically based on a variety of prognostic parameters and markers (Fitzgibbons et al., 2000, Arch. Pathol. Lab. Med. 124:966-978; Hamilton and Piccart, 2000, Ann. Oncol. 11:647-663), including genetic predispostion markers BRCA-1 and BRCA-2 (Robson, 2000, J. Clin. Oncol. 18:113sup-118sup).

The identification of novel therapeutic targets and diagnostic markers is essential for improving the current treatment of ovarian cancer patients. Recent advances in molecular medicine have increased the interest in tumor-specific cell surface antigens that could serve as targets for various immunotherapeutic or small molecule strategies. Antigens suitable for immunotherapeutic strategies should be highly expressed in cancer tissues and ideally not expressed in normal adult tissues. Expression in tissues that are dispensable for life, however, may be tolerated. Examples of such antigens include Her2/neu and the B-cell antigen CD20. Humanized monclonal antibodies directed to Her2/neu (Herceptin®/trastuzumab) are currently in use for the treatment of metastatic breast cancer (Ross and Fletcher, 1998, Stem Cells 16:413-428). Similarly, anti-CD20 monoclonal antibodies (Rituxin®/rituximab) are used to effectively treat non-Hodgekin's lymphoma (Maloney et al., 1997, Blood 90:2188-2195; Leget and Czuczman, 1998, Curr. Opin. Oncol. 10:548-551).

Potential immunotherapeutic targets have been identified for ovarian cancer. One such target is polymorphic epithelial mucin (MUC1). MUC1 is a transmembrane protein, present at the apical surface of glandular epithelial cells. It is often overexpressed in ovarian cancer, and typically exhibits an altered glycosylation pattern, resulting in an antigenically distinct molecule, and is in early clinical trials as a vaccine target (Gilewski et al., 2000, Clin. Cancer Res. 6:1693-1701; Scholl et al., 2000, J. Immunother. 23:570-580). The tumor-expressed protein is often cleaved into the circulation, where it is detectable as the tumor marker, CA 15-3 (Bon et al., 1997, Clin. Chem. 43:585-593). However, many patients have tumors that express neither HER2 nor MUC-1; therefore, it is clear that other targets need to be identified to manage localized and metastatic disease.

Mutations in both BRCA1 and BRCA2 are associated with increased susceptibility to ovarian cancer. Mutations in BRCA1 occur in approximately 5 percent (95 percent confidence interval, 3 to 8 percent) of women in whom ovarian cancer is diagnosed before the age of 70 years (John F. Stratton et al. (1997) N Engl J. Med. 336:1125-1130). And, in BRCA1 gene carriers, the risk for developing ovarian cancer is 0.63 (Am J. Hum Genet 56:267, 1995).

Other biochemical markers such as CA125 have been reported to be associated with ovarian cancer, but they are not absolute indicators of disease. Although roughly 85% of women with clinically apparent ovarian cancer have increased levels of CA125, CA125 is also increased during the first trimester of pregnancy, during menstruation, in the presence of non-cancerous illnesses and in cancers of other sites.

While industry and academia have identified novel sequences, there has not been an equal effort exerted to identify the function of these novel sequences. The elucidation of a role for novel proteins and compounds in disease states for identification of therapeutic targets and diagnostic markers is essential for improving the current treatment of ovarian cancer patients. Accordingly, provided herein are molecular targets for therapeutic intervention in ovarian and other cancers. Additionally, provided herein are methods that can be used in diagnosis and prognosis of ovarian cancer. Further provided are methods that can be used to screen candidate bioactive agents for the ability to modulate ovarian cancer.

### SUMMARY OF THE INVENTION

The present invention therefore provides nucleotide sequences of genes that are up- and down-regulated in ovarian cancer cells. Such genes are useful for diagnostic purposes, and also as targets for screening for therapeutic compounds that modulate ovarian cancer, such as hormones or antibodies. Other aspects of the invention will become apparent to the skilled artisan by the following description of the invention.

In one aspect, the present invention provides a method of detecting a ovarian cancer-associated transcript in a cell from a patient, the method comprising contacting a biological sample from the patient with a polynucleotide that selectively hybridizes to a sequence at least 80% identical to a sequence as shown in Tables 1-6.

In one embodiment, the present invention provides a method of determining the level of a ovarian cancer associated transcript in a cell from a patient.

In one embodiment, the present invention provides a method of detecting a ovarian cancer-associated transcript in a cell from a patient, the method comprising contacting a biological sample from the patient with a polynucleotide that selectively hybridizes to a sequence at least 80% identical to a sequence as shown in Tables 1-6.

In one embodiment, the polynucleotide selectively hybridizes to a sequence at least 95% identical to a sequence as shown in Tables 1-6.

In one embodiment, the biological sample is a tissue sample. In another embodiment, the biological sample comprises isolated nucleic acids, e.g., mRNA.

In one embodiment, the polynucleotide is labeled, e.g., with a fluorescent label.

In one embodiment, the polynucleotide is immobilized on a solid surface.

In one embodiment, the patient is undergoing a therapeutic regimen to treat ovarian cancer. In another embodiment, the patient is suspected of having metastatic ovarian cancer.

In one embodiment, the patient is a human.

In one embodiment, the ovarian cancer associated transcript is mRNA.

In one embodiment, the method further comprises the step of amplifying nucleic acids before the step of contacting the biological sample with the polynucleotide.

In another aspect, the present invention provides a method of monitoring the efficacy of a therapeutic treatment of ovarian cancer, the method comprising the steps of: (i) providing a biological sample from a patient undergoing the therapeutic treatment; and (ii) determining the level of a ovarian cancer-associated transcript in the biological sample by contacting the biological sample with a polynucleotide that selectively hybridizes to a sequence at least 80% identical to a sequence as shown in Tables 1-6, thereby monitoring the efficacy of the therapy. In a further embodiment, the patient has metastatic ovarian cancer. In a further embodiment, the patient has a drug resistant form of ovarian cancer.

In one embodiment, the method further comprises the step of: (iii) comparing the level of the ovarian cancer-associated transcript to a level of the ovarian cancer-associated transcript in a biological sample from the patient prior to, or earlier in, the therapeutic treatment.

Additionally, provided herein is a method of evaluating the effect of a candidate ovarian cancer drug comprising administering the drug to a patient and removing a cell sample from the patient. The expression profile of the cell is then determined. This method may further comprise comparing the expression profile to an expression profile of a healthy individual. In a preferred embodiment, said expression profile includes a gene of Tables 1-6.

In one aspect, the present invention provides an isolated nucleic acid molecule consisting of a polynucleotide sequence as shown in Tables 1-6.

In one embodiment, an expression vector or cell comprises the isolated nucleic acid.

In one aspect, the present invention provides an isolated polypeptide which is encoded by a nucleic acid molecule having polynucleotide sequence as shown in Tables 1-6.

In another aspect, the present invention provides an antibody that specifically binds to an isolated polypeptide which is encoded by a nucleic acid molecule having polynucleotide sequence as shown in Tables 1-6.

In one embodiment, the antibody is conjugated to an effector component, e.g., a fluorescent label, a radioisotope or a cytotoxic chemical.

In one embodiment, the antibody is an antibody fragment. In another embodiment, the antibody is humanized.

In one aspect, the present invention provides a method of detecting a ovarian cancer cell in a biological sample from a patient, the method comprising contacting the biological sample with an antibody as described herein.

In another aspect, the present invention provides a method of detecting antibodies specific to ovarian cancer in a patient, the method comprising contacting a biological sample from the patient with a polypeptide encoded by a nucleic acid comprising a sequence from Tables 1-6.

In another aspect, the present invention provides a method for identifying a compound that modulates a ovarian cancer-associated polypeptide, the method comprising the steps of: (i) contacting the compound with a ovarian cancer-associated polypeptide, the polypeptide encoded by a polynucleotide that selectively hybridizes to a sequence at least 80% identical to a sequence as shown in Tables 1-6; and (ii) determining the functional effect of the compound upon the polypeptide.

In one embodiment, the functional effect is a physical effect, an enzymatic effect, or a chemical effect.

In one embodiment, the polypeptide is expressed in a eukaryotic host cell or cell membrane. In another embodiment, the polypeptide is recombinant.

In one embodiment, the functional effect is determined by measuring ligand binding to the polypeptide.

In another aspect, the present invention provides a method of inhibiting proliferation of a ovarian cancer-associated cell to treat ovarian cancer in a patient, the method comprising the step of administering to the subject a therapeutically effective amount of a compound identified as described herein.

In one embodiment, the compound is an antibody.

In another aspect, the present invention provides a drug screening assay comprising the steps of: (i) administering a test compound to a mammal having ovarian cancer or to a cell sample isolated therefrom; (ii) comparing the level of gene expression of a polynucleotide that selectively hybridizes to a sequence at least 80% identical to a sequence as shown in Tables 1-6 in a treated cell or mammal with the level of gene expression of the polynucleotide in a control cell sample or mammal, wherein a test compound that modulates the level of expression of the polynucleotide is a candidate for the treatment of ovarian cancer.

In one embodiment, the control is a mammal with ovarian cancer or a cell sample therefrom that has not been treated with the test compound. In another embodiment, the control is a normal cell or mammal.

In one embodiment, the test compound is administered in varying amounts or concentrations. In another embodiment, the test compound is administered for varying time periods. In another embodiment, the comparison can occur after addition or removal of the drug candidate.

In one embodiment, the levels of a plurality of polynucleotides that selectively hybridize to a sequence at least 80% identical to a sequence as shown in Tables 1-6 are individually compared to their respective levels in a control cell sample or mammal. In a preferred embodiment the plurality of polynucleotides is from three to ten.

In another aspect, the present invention provides a method for treating a mammal having ovarian cancer comprising administering a compound identified by the assay described herein.

In another aspect, the present invention provides a pharmaceutical composition for treating a mammal having ovarian cancer, the composition comprising a compound identified by the assay described herein and a physiologically acceptable excipient.

In one aspect, the present invention provides a method of screening drug candidates by providing a cell expressing a gene that is up- and down-regulated as in a ovarian cancer. In one embodiment, a gene is selected from Tables 1-6. The method further includes adding a drug candidate to the cell and determining the effect of the drug candidate on the expression of the expression profile gene.

In one embodiment, the method of screening drug candidates includes comparing the level of expression in the absence of the drug candidate to the level of expression in the presence of the drug candidate, wherein the concentration of the drug candidate can vary when present, and wherein the comparison can occur after addition or removal of the drug candidate. In a preferred embodiment, the cell expresses at least two expression profile genes. The profile genes may show an increase or decrease.

Also provided is a method of evaluating the effect of a candidate ovarian cancer drug comprising administering the drug to a transgenic animal expressing or over-expressing the ovarian cancer modulatory protein, or an animal lacking the ovarian cancer modulatory protein, for example as a result of a gene knockout.

Moreover, provided herein is a biochip comprising one or more nucleic acid segments of Tables 1-6, wherein the biochip comprises fewer than 1000 nucleic acid probes. Preferably, at least two nucleic acid segments are included. More preferably, at least three nucleic acid segments are included.

Furthermore, a method of diagnosing a disorder associated with ovarian cancer is provided. The method comprises determining the expression of a gene of Tables 1-6 in a first tissue type of a first individual, and comparing the distribution to the expression of the gene from a second normal tissue type from the first individual or a second unaffected individual. A difference in the expression indicates that the first individual has a disorder associated with ovarian cancer.

In a further embodiment, the biochip also includes a polynucleotide sequence of a gene that is not up- and down-regulated in ovarian cancer.

In one embodiment a method for screening for a bioactive agent capable of interfering with the binding of a ovarian cancer modulating protein (ovarian cancer modulatory protein) or a fragment thereof and an antibody which binds to said ovarian cancer modulatory protein or fragment thereof. In a preferred embodiment, the method comprises combining a ovarian cancer modulatory protein or fragment thereof, a candidate bioactive agent and an antibody which binds to said ovarian cancer modulatory protein or fragment thereof. The method further includes determining the binding of said ovarian cancer modulatory protein or fragment thereof and said antibody. Wherein there is a change in binding, an agent is identified as an interfering agent. The interfering agent can be an agonist or an antagonist. Preferably, the agent inhibits ovarian cancer.

Also provided herein are methods of eliciting an immune response in an individual. In one embodiment a method provided herein comprises administering to an individual a composition comprising a ovarian cancer modulating protein, or a fragment thereof. In another embodiment, the protein is encoded by a nucleic acid selected from those of Tables 1-6.

Further provided herein are compositions capable of eliciting an immune response in an individual. In one embodiment, a composition provided herein comprises a ovarian cancer modulating protein, preferably encoded by a nucleic acid of Table 1-6 or a fragment thereof, and a pharmaceutically acceptable carrier. In another embodiment, said composition comprises a nucleic acid comprising a sequence encoding a ovarian cancer modulating protein, preferably selected from the nucleic acids of Tables 1-6, and a pharmaceutically acceptable carrier.

Also provided are methods of neutralizing the effect of a ovarian cancer protein, or a fragment thereof, comprising contacting an agent specific for said protein with said protein in an amount sufficient to effect neutralization. In another embodiment, the protein is encoded by a nucleic acid selected from those of Tables 1-6.

In another aspect of the invention, a method of treating an individual for ovarian cancer is provided. In one embodiment, the method comprises administering to said individual an inhibitor of a ovarian cancer modulating protein. In another embodiment, the method comprises administering to a patient having ovarian cancer an antibody to a ovarian cancer modulating protein conjugated to a therapeutic moiety. Such a therapeutic moiety can be a cytotoxic agent or a radioisotope.

### DETAILED DESCRIPTION OF THE INVENTION

In accordance with the objects outlined above, the present invention provides novel methods for diagnosis and prognosis evaluation for ovarian cancer (PC), including metastatic ovarian cancer, as well as methods for screening for compositions which modulate ovarian cancer. Also provided are methods for treating ovarian cancer.

Tables 1-6 provide unigene cluster identification numbers for the nucleotide sequence of genes that exhibit increased or decreased expression in ovarian cancer samples. Tables 1-6 also provide an exemplar accession number that provides a nucleotide sequence that is part of the unigene cluster.

Definitions

The term “ovarian cancer protein” or “ovarian cancer polynucleotide” or “ovarian cancer-associated transcript” refers to nucleic acid and polypeptide polymorphic variants, alleles, mutants, and interspecies homologues that: (1) have a nucleotide sequence that has greater than about 60% nucleotide sequence identity, 65%, 70%, 75%, 80%, 85%, 90%, preferably 91%, 92%, 93%, 94%, 95%, 96%, 97%, 98% or 99% or greater nucleotide sequence identity, preferably over a region of over a region of at least about 25, 50, 100, 200, 500, 1000, or more nucleotides, to a nucleotide sequence of or associated with a gene of Tables 1-6; (2) bind to antibodies, e.g., polyclonal antibodies, raised against an immunogen comprising an amino acid sequence encoded by a nucleotide sequence of or associated with a gene of Tables 1-6, and conservatively modified variants thereof; (3) specifically hybridize under stringent hybridization conditions to a nucleic acid sequence, or the complement thereof of Tables 1-6 and conservatively modified variants thereof or (4) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino sequence identity, preferably over a region of over a region of at least about 25, 50, 100, 200, 500, 1000, or more amino acid, to an amino acid sequence encoded by a nucleotide sequence of or associated with a gene of Tables 1-6. A polynucleotide or polypeptide sequence is typically from a mammal including, but not limited to, primate, e.g., human; rodent, e.g., rat, mouse, hamster; cow, pig, horse, sheep, or other mammal. A “ovarian cancer polypeptide” and a “ovarian cancer polynucleotide,” include both naturally occurring or recombinant forms.

A “full length” ovarian cancer protein or nucleic acid refers to a ovarian cancer polypeptide or polynucleotide sequence, or a variant thereof, that contains all of the elements normally contained in one or more naturally occurring, wild type ovarian cancer polynucleotide or polypeptide sequences. The “full length” may be prior to, or after, various stages of post-translation processing or splicing, including alternative splicing.

“Biological sample” as used herein is a sample of biological tissue or fluid that contains nucleic acids or polypeptides, e.g., of a ovarian cancer protein, polynucleotide or transcript. Such samples include, but are not limited to, tissue isolated from primates, e.g., humans, or rodents, e.g., mice, and rats. Biological samples may also include sections of tissues such as biopsy and autopsy samples, frozen sections taken for histologic purposes, blood, plasma, serum, sputum, stool, tears, mucus, hair, skin, etc. Biological samples also include explants and primary and/or transformed cell cultures derived from patient tissues. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

“Providing a biological sample” means to obtain a biological sample for use in methods described in this invention. Most often, this will be done by removing a sample of cells from an animal, but can also be accomplished by using previously isolated cells (e.g., isolated by another person, at another time, and/or for another purpose), or by performing the methods of the invention in vivo. Archival tissues, having treatment or outcome history, will be particularly useful.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

A “host cell” is a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as CHO, HeLa, and the like (see, e.g., the American Type Culture Collection catalog or web site, www.atcc.org).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.typically conservative substitutions for one another: 1) Alanine (A), Glycine (G); 2) Aspartic acid (D), Glutamic acid (E); 3) Asparagine (N), Glutamine (O); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor & Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. A nucleic acid of the present invention will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g. to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A variety of references disclose such nucleic acid analogs, including, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched basepairs. DNA and RNA typically exhibit a 2-4° C. drop in Tₘ for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each containing a base, are referred to herein as a nucleoside.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The labels may be incorporated into the ovarian cancer nucleic acids, proteins and antibodies at any position. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effector” can be a variety of molecules including, e.g., detection moieties including radioactive compounds, fluorescent compounds, an enzyme or substrate, tags such as epitope tags, a toxin; activatable moieties, a chemotherapeutic agent; a lipase; an antibiotic; or a radioisotope emitting “hard” e.g., beta radiation.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, method using high affinity interactions may achieve the same results where one of a pair of binding partners binds to the other, e.g., biotin, streptavidin.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not functionally interfere with hybridization. Thus, e.g., probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence. Diagnosis or prognosis may be based at the genomic level, or at the level of RNA or protein expression.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

Nucleic acids that do not hybridize to each other under stringent conditions are still substantially identical if the polypeptides which they encode are substantially identical. This occurs, e.g., when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

The phrase “functional effects” in the context of assays for testing compounds that modulate activity of a ovarian cancer protein includes the determination of a parameter that is indirectly or directly under the influence of the ovarian cancer protein or nucleic acid, e.g., a functional, physical, or chemical effect, such as the ability to decrease ovarian cancer. It includes ligand binding activity; cell growth on soft agar; anchorage dependence; contact inhibition and density limitation of growth; cellular proliferation; cellular transformation; growth factor or serum dependence; tumor specific marker levels; invasiveness into Matrigel; tumor growth and metastasis in vivo; mRNA and protein expression in cells undergoing metastasis, and other characteristics of ovarian cancer cells. “Functional effects” include in vitro, in vivo, and ex vivo activities.

By “determining the functional effect” is meant assaying for a compound that increases or decreases a parameter that is indirectly or directly under the influence of a ovarian cancer protein sequence, e.g., functional, enzymatic, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of the ovarian cancer protein; measuring binding activity or binding assays, e.g. binding to antibodies or other ligands, and measuring cellular proliferation. Determination of the functional effect of a compound on ovarian cancer can also be performed using ovarian cancer assays known to those of skill in the art such as an in vitro assays, e.g., cell growth on soft agar; anchorage dependence; contact inhibition and density limitation of growth; cellular proliferation; cellular transformation; growth factor or serum dependence; tumor specific marker levels; invasiveness into Matrigel; tumor growth and metastasis in vivo; mRNA and protein expression in cells undergoing metastasis, and other characteristics of ovarian cancer cells. The functional effects can be evaluated by many means known to those skilled in the art, e.g., microscopy for quantitative or qualitative measures of alterations in morphological features, measurement of changes in RNA or protein levels for ovarian cancer-associated sequences, measurement of RNA stability, identification of downstream or reporter gene expression (CAT, luciferase, β-gal, GFP and the like), e.g., via chemiluminescence, fluorescence, colorimetric reactions, antibody binding, inducible markers, and ligand binding assays.

“Inhibitors”, “activators”, and “modulators” of ovarian cancer polynucleotide and polypeptide sequences are used to refer to activating, inhibitory, or modulating molecules or compounds identified using in vitro and in vivo assays of ovarian cancer polynucleotide and polypeptide sequences. Inhibitors are compounds that, e.g., bind to, partially or totally block activity, decrease, prevent, delay activation, inactivate, desensitize, or down regulate the activity or expression of ovarian cancer proteins, e.g., antagonists. Antisense nucleic acids may seem to inhibit expression and subsequent function of the protein. “Activators” are compounds that increase, open, activate, facilitate, enhance activation, sensitize, agonize, or up regulate ovarian cancer protein activity. Inhibitors, activators, or modulators also include genetically modified versions of ovarian cancer proteins, e.g., versions with altered activity, as well as naturally occurring and synthetic ligands, antagonists, agonists, antibodies, small chemical molecules and the like. Such assays for inhibitors and activators include, e.g., expressing the ovarian cancer protein in vitro, in cells, or cell membranes, applying putative modulator compounds, and then determining the functional effects on activity, as described above. Activators and inhibitors of ovarian cancer can also be identified by incubating ovarian cancer cells with the test compound and determining increases or decreases in the expression of 1 or more ovarian cancer proteins, e.g., 1, 2, 3, 4, 5, 10, 15, 20, 25, 30, 40, 50 or more ovarian cancer proteins, such as ovarian cancer proteins encoded by the sequences set out in Tables 1-6.

Samples or assays comprising ovarian cancer proteins that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of a polypeptide is achieved when the activity value relative to the control is about 80%, preferably 50%, more preferably 25-0%. Activation of a ovarian cancer polypeptide is achieved when the activity value relative to the control (untreated with activators) is 110%, more preferably 150%, more preferably 200-500% (i.e., two to five fold higher relative to the control), more preferably 1000-3000% higher.

The phrase “changes in cell growth” refers to any change in cell growth and proliferation characteristics in vitro or in vivo, such as formation of foci, anchorage independence, semi-solid or soft agar growth, changes in contact inhibition and density limitation of growth, loss of growth factor or serum requirements, changes in cell morphology, gaining or losing immortalization, gaining or losing tumor specific markers, ability to form or suppress tumors when injected into suitable animal hosts, and/or immortalization of the cell. See, e.g., Freshney, Culture of Animal Cells a Manual of Basic Technique pp. 231-241 (3^{rd} ed. 1994).

“Tumor cell” refers to precancerous, cancerous, and normal cells in a tumor.

“Cancer cells,” “transformed” cells or “transformation” in tissue culture, refers to spontaneous or induced phenotypic changes that do not necessarily involve the uptake of new genetic material. Although transformation can arise from infection with a transforming virus and incorporation of new genomic DNA, or uptake of exogenous DNA, it can also arise spontaneously or following exposure to a carcinogen, thereby mutating an endogenous gene. Transformation is associated with phenotypic changes, such as immortalization of cells, aberrant growth control, nonmorphological changes, and/or malignancy (see, Freshney, Culture of Animal Cells a Manual of Basic Technique (3^{rd} ed. 1994)).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody or its functional equivalent will be most critical in specificity and affinity of binding. See Paul, Fundamental Immunology.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, e.g., pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985); Coligan, Current Protocols in Immunology (1991); Harlow & Lane, Antibodies, A Laboratory Manual (1988); and Goding, Monoclonal Antibodies: Principles and Practice (2d ed. 1986)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

Identification of Ovarian Cancer-Associated Sequences

In one aspect, the expression levels of genes are determined in different patient samples for which diagnosis information is desired, to provide expression profiles. An expression profile of a particular sample is essentially a “fingerprint” of the state of the sample; while two states may have any particular gene similarly expressed, the evaluation of a number of genes simultaneously allows the generation of a gene expression profile that is characteristic of the state of the cell. That is, normal tissue (e.g., normal ovarian or other tissue) may be distinguished from cancerous or metastatic cancerous tissue of the ovarian, or ovarian cancer tissue or metastatic ovarian cancerous tissue can be compared with tissue samples of ovarian and other tissues from surviving cancer patients. By comparing expression profiles of tissue in known different ovarian cancer states, information regarding which genes are important (including both up- and down-regulation of genes) in each of these states is obtained.

The identification of sequences that are differentially expressed in ovarian cancer versus non-ovarian cancer tissue allows the use of this information in a number of ways. For example, a particular treatment regime may be evaluated: does a chemotherapeutic drug act to down-regulate ovarian cancer, and thus tumor growth or recurrence, in a particular patient. Similarly, diagnosis and treatment outcomes may be done or confirmed by comparing patient samples with the known expression profiles. Metastatic tissue can also be analyzed to determine the stage of ovarian cancer in the tissue. Furthermore, these gene expression profiles (or individual genes) allow screening of drug candidates with an eye to mimicking or altering a particular expression profile; e.g., screening can be done for drugs that suppress the ovarian cancer expression profile. This may be done by making biochips comprising sets of the important ovarian cancer genes, which can then be used in these screens. These methods can also be done on the protein basis; that is, protein expression levels of the ovarian cancer proteins can be evaluated for diagnostic purposes or to screen candidate agents. In addition, the ovarian cancer nucleic acid sequences can be administered for gene therapy purposes, including the administration of antisense nucleic acids, or the ovarian cancer proteins (including antibodies and other modulators thereof) administered as therapeutic drugs.

Thus the present invention provides nucleic acid and protein sequences that are differentially expressed in ovarian cancer, herein termed “ovarian cancer sequences.” As outlined below, ovarian cancer sequences include those that are up-regulated (i.e., expressed at a higher level) in ovarian cancer, as well as those that are down-regulated (i.e., expressed at a lower level). In a preferred embodiment, the ovarian cancer sequences are from humans; however, as will be appreciated by those in the art, ovarian cancer sequences from other organisms may be useful in animal models of disease and drug evaluation; thus, other ovarian cancer sequences are provided, from vertebrates, including mammals, including rodents (rats, mice, hamsters, guinea pigs, etc.), primates, farm animals (including sheep, goats, pigs, cows, horses, etc.) and pets, e.g., (dogs, cats, etc.). Ovarian cancer sequences from other organisms may be obtained using the techniques outlined below.

Ovarian cancer sequences can include both nucleic acid and amino acid sequences. As will be appreciated by those in the art and is more fully outlined below, ovarian cancer nucleic acid sequences are useful in a variety of applications, including diagnostic applications, which will detect naturally occurring nucleic acids, as well as screening applications; e.g., biochips comprising nucleic acid probes or PCR microtiter plates with selected probes to the ovarian cancer sequences can be generated.

A ovarian cancer sequence can be initially identified by substantial nucleic acid and/or amino acid sequence homology to the ovarian cancer sequences outlined herein. Such homology can be based upon the overall nucleic acid or amino acid sequence, and is generally determined as outlined below, using either homology programs or hybridization conditions.

For identifying ovarian cancer-associated sequences, the ovarian cancer screen typically includes comparing genes identified in different tissues, e.g., normal and cancerous tissues, or tumor tissue samples from patients who have metastatic disease vs. non metastatic tissue. Other suitable tissue comparisons include comparing ovarian cancer samples with metastatic cancer samples from other cancers, such as lung, ovarian, gastrointestinal cancers, ovarian, etc. Samples of different stages of ovarian cancer, e.g., survivor tissue, drug resistant states, and tissue undergoing metastasis, are applied to biochips comprising nucleic acid probes. The samples are first microdissected, if applicable, and treated as is known in the art for the preparation of mRNA. Suitable biochips are commercially available, e.g. from Affymetrix. Gene expression profiles as described herein are generated and the data analyzed.

In one embodiment, the genes showing changes in expression as between normal and disease states are compared to genes expressed in other normal tissues, preferably normal ovarian, but also including, and not limited to lung, heart, brain, liver, ovarian, kidney, muscle, colon, small intestine, large intestine, spleen, bone and placenta. In a preferred embodiment, those genes identified during the ovarian cancer screen that are expressed in any significant amount in other tissues are removed from the profile, although in some embodiments, this is not necessary. That is, when screening for drugs, it is usually preferable that the target be disease specific, to minimize possible side effects.

In a preferred embodiment, ovarian cancer sequences are those that are up-regulated in ovarian cancer; that is, the expression of these genes is higher in the ovarian cancer tissue as compared to non-cancerous tissue. “Up-regulation” as used herein often means at least about a two-fold change, preferably at least about a three fold change, with at least about five-fold or higher being preferred. All unigene cluster identification numbers and accession numbers herein are for the GenBank sequence database and the sequences of the accession numbers are hereby expressly incorporated by reference. GenBank is known in the art, see, e.g., Benson, DA, et al., Nucleic Acids Research 26:1-7 (1998) and http://www.ncbi.nlm.nih.gov/. Sequences are also available in other databases, e.g., European Molecular Biology Laboratory (EMBL) and DNA Database of Japan (DDBJ). U.S. patent application Ser. No. 09/687,576, with the same assignee as the present application, further discloses related sequences, compositions, and methods of diagnosis and treatment of ovarian cancer is hereby expressly incorporated by reference.

In another preferred embodiment, ovarian cancer sequences are those that are down-regulated in the ovarian cancer; that is, the expression of these genes is lower in ovarian cancer tissue as compared to non-cancerous tissue. “Down-regulation” as used herein often means at least about a two-fold change, preferably at least about a three fold change, with at least about five-fold or higher being preferred.

Informatics

The ability to identify genes that are over or under expressed in ovarian cancer can additionally provide high-resolution, high-sensitivity datasets which can be used in the areas of diagnostics, therapeutics, drug development, pharmacogenetics, protein structure, biosensor development, and other related areas. For example, the expression profiles can be used in diagnostic or prognostic evaluation of patients with ovarian cancer. Or as another example, subcellular toxicological information can be generated to better direct drug structure and activity correlation (see Anderson, Pharmaceutical Proteomics: Targets, Mechanism, and Function, paper presented at the IBC Proteomics conference, Coronado, Calif. (Jun. 11-12, 1998)). Subcellular toxicological information can also be utilized in a biological sensor device to predict the likely toxicological effect of chemical exposures and likely tolerable exposure thresholds (see U.S. Pat. No. 5,811,231). Similar advantages accrue from datasets relevant to other biomolecules and bioactive agents (e.g., nucleic acids, saccharides, lipids, drugs, and the like).

Thus, in another embodiment, the present invention provides a database that includes at least one set of assay data. The data contained in the database is acquired, e.g., using array analysis either singly or in a library format. The database can be in substantially any form in which data can be maintained and transmitted, but is preferably an electronic database. The electronic database of the invention can be maintained on any electronic device allowing for the storage of and access to the database, such as a personal computer, but is preferably distributed on a wide area network, such as the World Wide Web.

The focus of the present section on databases that include peptide sequence data is for clarity of illustration only. It will be apparent to those of skill in the art that similar databases can be assembled for any assay data acquired using an assay of the invention.

The compositions and methods for identifying and/or quantitating the relative and/or absolute abundance of a variety of molecular and macromolecular species from a biological sample undergoing ovarian cancer, i.e., the identification of ovarian cancer-associated sequences described herein, provide an abundance of information, which can be correlated with pathological conditions, predisposition to disease, drug testing, therapeutic monitoring, gene-disease causal linkages, identification of correlates of immunity and physiological status, among others. Although the data generated from the assays of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures.

See also Mount et al., Bioinformatics (2001); Biological Sequence Analysis: Probabilistic Models of Proteins and Nucleic Acids (Durbin et al., eds., 1999); Bioinformatics: A Practical Guide to the Analysis of Genes and Proteins (Baxevanis & Oeullette eds., 1998)); Rashidi & Buehler, Bioinformatics: Basic Applications in Biological Science and Medicine (1999); Introduction to Computational Molecular Biology (Setubal et al., eds 1997); Bioinformatics: Methods and Protocols (Misener & Krawetz, eds, 2000); Bioinformatics: Sequence, Structure, and Databanks: A Practical Approach (Higgins & Taylor, eds., 2000); Brown, Bioinformatics: A Biologist's Guide to Biocomputing and the Internet (2001); Han & Kamber, Data Mining: Concepts and Techniques (2000); and Waterman, Introduction to Computational Biology: Maps, Sequences, and Genomes (1995).

The present invention provides a computer database comprising a computer and software for storing in computer-retrievable form assay data records cross-tabulated, e.g., with data specifying the source of the target-containing sample from which each sequence specificity record was obtained.

In an exemplary embodiment, at least one of the sources of target-containing sample is from a control tissue sample known to be free of pathological disorders. In a variation, at least one of the sources is a known pathological tissue specimen, e.g., a neoplastic lesion or another tissue specimen to be analyzed for ovarian cancer. In another variation, the assay records cross-tabulate one or more of the following parameters for each target species in a sample: (1) a unique identification code, which can include, e.g., a target molecular structure and/or characteristic separation coordinate (e.g., electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a peptide or nucleic acid, the invention preferably provides a method for identifying related peptide or nucleic acid sequences, comprising performing a computerized comparison between a peptide or nucleic acid sequence assay record stored in or retrieved from a computer storage device or database and at least one other sequence. The comparison can include a sequence analysis or comparison algorithm or computer program embodiment thereof (e.g., FASTA, TFASTA, GAP, BESTFIT) and/or the comparison may be of the relative amount of a peptide or nucleic acid sequence in a pool of sequences determined from a polypeptide or nucleic acid sample of a specimen.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program can be transferred to secondary memory, which is typically random access memory (e.g., DRAM, SRAM, SGRAM, or SDRAM). Targets are ranked according to the degree of correspondence between a selected assay characteristic (e.g., binding to a selected affinity moiety) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison target, such as a query target; and (4) a program for alignment and comparison, typically with rank-ordering of comparison results on the basis of computed similarity values.

Characteristics of Ovarian Cancer-Associated Proteins

Ovarian cancer proteins of the present invention may be classified as secreted proteins, transmembrane proteins or intracellular proteins. In one embodiment, the ovarian cancer protein is an intracellular protein. Intracellular proteins may be found in the cytoplasm and/or in the nucleus. Intracellular proteins are involved in all aspects of cellular function and replication (including, e.g., signaling pathways); aberrant expression of such proteins often results in unregulated or disregulated cellular processes (see, e.g., Molecular Biology of the Cell (Alberts, ed., 3rd ed., 1994). For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate. One useful database is Pfam (protein families), which is a large collection of multiple sequence alignments and hidden Markov models covering many common protein domains. Versions are available via the internet from Washington University in St. Louis, the Sanger Center in England, and the Karolinska Institute in Sweden (see, e.g., Bateman et al., Nuc. Acids Res. 28:263-266 (2000); Sonnhammer et al., Proteins 28:405-420 (1997); Bateman et al., Nuc. Acids Res. 27:260-262 (1999); and Sonnhammer et al., Nuc. Acids Res. 26:320-322-(1998)).

In another embodiment, the ovarian cancer sequences are transmembrane proteins. Transmembrane proteins are molecules that span a phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors such as G protein coupled receptors (GPCRs) are classified as “seven transmembrane domain” proteins, as they contain 7 membrane spanning regions. Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted (see, e.g. PSORT web site http://psort.nibb.ac.jp/). Important transmembrane protein receptors include, but are not limited to the insulin receptor, insulin-like growth factor receptor, human growth hormone receptor, glucose transporters, transferrin receptor, epidermal growth factor receptor, low density lipoprotein receptor, epidermal growth factor receptor, leptin receptor, interleukin receptors, e.g. IL-1 receptor, IL-2 receptor,

The extracellular domains of transmembrane proteins are diverse; however, conserved motifs are found repeatedly among various extracellular domains. Conserved structure and/or functions have been ascribed to different extracellular motifs. Many extracellular domains are involved in binding to other molecules. In one aspect, extracellular domains are found on receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands can be tethered to the cell, e.g., via a glycosylphosphatidylinositol (GPI) anchor, or may themselves be transmembrane proteins. Extracellular domains also associate with the extracellular matrix and contribute to the maintenance of the cell structure.

Ovarian cancer proteins that are transmembrane are particularly preferred in the present invention as they are readily accessible targets for immunotherapeutics, as are described herein. In addition, as outlined below, transmembrane proteins can be also useful in imaging modalities. Antibodies may be used to label such readily accessible proteins in situ. Alternatively, antibodies can also label intracellular proteins, in which case samples are typically permeablized to provide access to intracellular proteins.

It will also be appreciated by those in the art that a transmembrane protein can be made soluble by removing transmembrane sequences, e.g., through recombinant methods. Furthermore, transmembrane proteins that have been made soluble can be made to be secreted through recombinant means by adding an appropriate signal sequence.

In another embodiment, the ovarian cancer proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. Ovarian cancer proteins that are secreted proteins are particularly preferred in the present invention as they serve as good targets for diagnostic markers, e.g., for blood, plasma, serum, or stool tests.

Use of Ovarian Cancer Nucleic Acids

As described above, ovarian cancer sequence is initially identified by substantial nucleic acid and/or amino acid sequence homology or linkage to the ovarian cancer sequences outlined herein. Such homology can be based upon the overall nucleic acid or amino acid sequence, and is generally determined as outlined below, using either homology programs or hybridization conditions. Typically, linked sequences on a mRNA are found on the same molecule.

The ovarian cancer nucleic acid sequences of the invention, e.g., the sequences in Table 1-6, can be fragments of larger genes, i.e., they are nucleic acid segments. “Genes” in this context includes coding regions, non-coding regions, and mixtures of coding and non-coding regions. Accordingly, as will be appreciated by those in the art, using the sequences provided herein, extended sequences, in either direction, of the ovarian cancer genes can be obtained, using techniques well known in the art for cloning either longer sequences or the full length sequences; see Ausubel, et al., supra. Much can be done by informatics and many sequences can be clustered to include multiple sequences corresponding to a single gene, e.g., systems such as UniGene (see, http://www.ncbi.nlm.nih.gov/UniGene/).

Once the ovarian cancer nucleic acid is identified, it can be cloned and, if necessary, its constituent parts recombined to form the entire ovarian cancer nucleic acid coding regions or the entire mRNA sequence. Once isolated from its natural source, e.g., contained within a plasmid or other vector or excised therefrom as a linear nucleic acid segment, the recombinant ovarian cancer nucleic acid can be further-used as a probe to identify and isolate other ovarian cancer nucleic acids, e.g., extended coding regions. It can also be used as a “precursor” nucleic acid to make modified or variant ovarian cancer nucleic acids and proteins.

The ovarian cancer nucleic acids of the present invention are used in several ways. In a first embodiment, nucleic acid probes to the ovarian cancer nucleic acids are made and attached to biochips to be used in screening and diagnostic methods, as outlined below, or for administration, e.g., for gene therapy, vaccine, and/or antisense applications. Alternatively, the ovarian cancer nucleic acids that include coding regions of ovarian cancer proteins can be put into expression vectors for the expression of ovarian cancer proteins, again for screening purposes or for administration to a patient.

In a preferred embodiment, nucleic acid probes to ovarian cancer nucleic acids (both the nucleic acid sequences outlined in the figures and/or the complements thereof) are made. The nucleic acid probes attached to the biochip are designed to be substantially complementary to the ovarian cancer nucleic acids, i.e. the target sequence (either the target sequence of the sample or to other probe sequences, e.g., in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally whole genes are not used. In some embodiments, much longer nucleic acids can be used, up to hundreds of bases.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e., have some sequence in common), or separate. In some cases, PCR primers may be used to amplify signal for higher sensitivity.

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can typically be covalent or non-covalent. By “non-covalent binding” and grammatical equivalents herein is meant one or more of electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant a material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, etc. In general, the substrates allow optical detection and do not appreciably fluoresce. A preferred substrate is described in copending application entitled Reusable Low Fluorescent Plastic Biochip, U.S. application Ser. No. 09/270,214, filed Mar. 15, 1999, herein incorporated by reference in its entirety.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In a preferred embodiment, the surface of the biochip and the probe may be derivatized with chemical functional groups for subsequent attachment of the two. Thus, e.g., the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, e.g. using linkers as are known in the art; e.g., homo-or hetero-bifunctional linkers as are well known (see 1994 Pierce Chemical Company catalog, technical section on cross-linkers, pages 155-200). In addition, in some cases, additional linkers, such as alkyl groups (including substituted and heteroalkyl groups) may be used.

In this embodiment, oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In another embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affimetrix GeneChip™ technology.

Often, amplification-based assays are performed to measure the expression level of ovarian cancer-associated sequences. These assays are typically performed in conjunction with reverse transcription. In such assays, a ovarian cancer-associated nucleic acid sequence acts as a template in an amplification reaction (e.g., Polymerase Chain Reaction, or PCR). In a quantitative amplification, the amount of amplification product will be proportional to the amount of template in the original sample. Comparison to appropriate controls provides a measure of the amount of ovarian cancer-associated RNA. Methods of quantitative amplification are well known to those of skill in the art. Detailed protocols for quantitative PCR are provided, e.g., in Innis et al., PCR Protocols, A Guide to Methods and Applications (1990).

In some embodiments, a TaqMan based assay is used to measure expression. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, e.g., literature provided by Perkin-Elmer, e.g., www2.perkin-elmer.com).

Other suitable amplification methods include, but are not limited to, ligase chain reaction (LCR) (see Wu & Wallace, Genomics 4:560 (1989), Landegren et al., Science 241:1077 (1988), and Barringer et al., Gene 89:117 (1990)), transcription amplification (Kwoh et al., Proc. Natl. Acad. Sci. USA 86:1173 (1989)), self-sustained sequence replication (Guatelli et al., Proc. Nat. Acad. Sci. USA 87:1874 (1990)), dot PCR, and linker adapter PCR, etc.

Expression of Ovarian Cancer Proteins from Nucleic Acids

In a preferred embodiment, ovarian cancer nucleic acids, e.g., encoding ovarian cancer proteins are used to make a variety of expression vectors to express ovarian cancer proteins which can then be used in screening assays, as described below. Expression vectors and recombinant DNA technology are well known to those of skill in the art (see, e.g., Ausubel, supra, and Gene Expression Systems (Fernandez & Hoeffler, eds, 1999)) and are used to express proteins. The expression vectors may be either self-replicating extrachromosomal vectors or vectors which integrate into a host genome. Generally, these expression vectors include transcriptional and translational regulatory nucleic acid operably linked to the nucleic acid encoding the ovarian cancer protein. The term “control sequences” refers to DNA sequences used for the expression of an operably linked coding sequence in a particular host organism. Control sequences that are suitable for prokaryotes, e.g., include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is typically accomplished by ligation at convenient restriction sites. If such sites do not exist, synthetic oligonucleotide adaptors or linkers are used in accordance with conventional practice. Transcriptional and translational regulatory nucleic acid will generally be appropriate to the host cell used to express the ovarian cancer protein. Numerous types of appropriate expression vectors, and suitable regulatory sequences are known in the art for a variety of host cells.

In general, transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, an expression vector may comprise additional elements. For example, the expression vector may have two replication systems, thus allowing it to be maintained in two organisms, e.g. in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art (e.g., Fernandez & Hoeffler, supra).

In addition, in a preferred embodiment, the expression vector contains a selectable marker gene to allow the selection of transformed host cells. Selection genes are well known in the art and will vary with the host cell used.

The ovarian cancer proteins of the present invention are produced by culturing a host cell transformed with an expression vector containing nucleic acid encoding a ovarian cancer protein, under the appropriate conditions to induce or cause expression of the ovarian cancer protein. Conditions appropriate for ovarian cancer protein expression will vary with the choice of the expression vector and the host cell, and will be easily ascertained by one skilled in the art through routine experimentation or optimization. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect and animal cells, including mammalian cells. Of particular interest are Saccharomyces cerevisiae and other yeasts, E. coli, Bacillus subtilis, Sf9 cells, C129 cells, 293 cells, Neurospora, BHK, CHO, COS, HeLa cells, HUVEC (human umbilical vein endothelial cells), THP1 cells (a macrophage cell line) and various other human cells and cell lines.

In a preferred embodiment, the ovarian cancer proteins are expressed in mammalian cells. Mammalian expression systems are also known in the art, and include retroviral and adenoviral systems. One expression vector system is a retroviral vector system such as is generally described in PCT/US97/01019 and PCT/US97/01048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter (see, e.g., Fernandez & Hoeffler, supra). Typically, transcription termination and polyadenylation sequences recognized by mammalian cells are regulatory regions located 3′ to the translation stop codon and thus, together with the promoter elements, flank the coding sequence. Examples of transcription terminator and polyadenlyation signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, ovarian cancer proteins are expressed in bacterial systems. Bacterial expression systems are well known in the art. Promoters from bacteriophage may also be used and are known in the art. In addition, synthetic promoters and hybrid promoters are also useful; e.g., the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the ovarian cancer protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others (e.g., Fernandez & Hoeffler, supra). The bacterial expression vectors are transformed into bacterial host cells using techniques well known in the art, such as calcium chloride treatment, electroporation, and others.

In one embodiment, ovarian cancer proteins are produced in insect cells. Expression vectors for the transformation of insect cells, and in particular, baculovirus-based expression vectors, are well known in the art.

In a preferred embodiment, ovarian cancer protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

The ovarian cancer protein may also be made as a fusion protein, using techniques well known in the art. Thus, e.g., for the creation of monoclonal antibodies, if the desired epitope is small, the ovarian cancer protein may be fused to a carrier protein to form an immunogen. Alternatively, the ovarian cancer protein may be made as a fusion protein to increase expression, or for other reasons. For example, when the ovarian cancer protein is a ovarian cancer peptide, the nucleic acid encoding the peptide may be linked to other nucleic acid for expression purposes.

In a preferred embodiment, the ovarian cancer protein is purified or isolated after expression. Ovarian cancer proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the ovarian cancer protein may be purified using a standard anti-ovarian cancer protein antibody column. Ultrafiltration and diafiltration techniques, in conjunction with protein concentration, are also useful. For general guidance in suitable purification techniques, see Scopes, Protein Purification (1982). The degree of purification necessary will vary depending on the use of the ovarian cancer protein. In some instances no purification will be necessary.

Once expressed and purified if necessary, the ovarian cancer proteins and nucleic acids are useful in a number of applications. They may be used as immunoselection reagents, as vaccine reagents, as screening agents, etc.

Variants of Ovarian Cancer Proteins

In one embodiment, the ovarian cancer proteins are derivative or variant ovarian cancer proteins as compared to the wild-type sequence. That is, as outlined more fully below, the derivative ovarian cancer peptide will often contain at least one amino acid substitution, deletion or insertion, with amino acid substitutions being particularly preferred. The amino acid substitution, insertion or deletion may occur at any residue within the ovarian cancer peptide.

Also included within one embodiment of ovarian cancer proteins of the present invention are amino acid sequence variants. These variants typically fall into one or more of three classes: substitutional, insertional or deletional variants. These variants ordinarily are prepared by site specific mutagenesis of nucleotides in the DNA encoding the ovarian cancer protein, using cassette or PCR mutagenesis or other techniques well known in the art, to produce DNA encoding the variant, and thereafter expressing the DNA in recombinant cell culture as outlined above. However, variant ovarian cancer protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the ovarian cancer protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed ovarian cancer variants screened for the optimal combination of desired activity. Techniques for making substitution mutations at predetermined sites in DNA having a known sequence are well known, e.g., M13 primer mutagenesis and PCR mutagenesis. Screening of the mutants is done using assays of ovarian cancer protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the ovarian cancer protein are desired, substitutions are generally made in accordance with the amino acid substitution relationships provided in the definition section.

The variants typically exhibit the same qualitative biological activity and will elicit the same immune response as the naturally-occurring analog, although variants also are selected to modify the characteristics of the ovarian cancer proteins as needed. Alternatively, the variant may be designed such that the biological activity of the ovarian cancer protein is altered. For example, glycosylation sites may be altered or removed.

Substantial changes in function or immunological identity are made by selecting substitutions that are less conservative than those described above. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

Covalent modifications of ovarian cancer polypeptides are included within the scope of this invention. One type of covalent modification includes reacting targeted amino acid residues of a ovarian cancer polypeptide with an organic derivatizing agent that is capable of reacting with selected side chains or the N-or C-terminal residues of a ovarian cancer polypeptide. Derivatization with bifunctional agents is useful, for instance, for crosslinking ovarian cancer polypeptides to a water-insoluble support matrix or surface for use in the method for purifying anti-ovarian cancer polypeptide antibodies or screening assays, as is more fully described below. Commonly used crosslinking agents include, e.g., 1,1-bis(diazoacetyl)-2-phenylethane, glutaraldehyde, N-hydroxysuccinimide esters, e.g., esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-((p-azidophenyl)dithio)propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the amino groups of the lysine, arginine, and histidine side chains (Creighton, Proteins: Structure and Molecular Properties, pp. 79-86 (1983)), acetylation of the N-terminal amine, and amidation of any C-terminal carboxyl group.

Another type of covalent modification of the ovarian cancer polypeptide included within the scope of this invention comprises altering the native glycosylation pattern of the polypeptide. “Altering the native glycosylation pattern” is intended for purposes herein to mean deleting one or more carbohydrate moieties found in native sequence ovarian cancer polypeptide, and/or adding one or more glycosylation sites that are not present in the native sequence ovarian cancer polypeptide. Glycosylation patterns can be altered in many ways. For example the use of different cell types to express ovarian cancer-associated sequences can result in different glycosylation patterns.

Addition of glycosylation sites to ovarian cancer polypeptides may also be accomplished by altering the amino acid sequence thereof. The alteration may be made, e.g., by the addition of, or substitution by, one or more serine or threonine residues to the native sequence ovarian cancer polypeptide (for O-linked glycosylation sites). The ovarian cancer amino acid sequence may optionally be altered through changes at the DNA level, particularly by mutating the DNA encoding the ovarian cancer polypeptide at preselected bases such that codons are generated that will translate into the desired amino acids.

Another means of increasing the number of carbohydrate moieties on the ovarian cancer polypeptide is by chemical or enzymatic coupling of glycosides to the polypeptide. Such methods are described in the art, e.g., in WO 87/05330, and in Aplin & Wriston, CRC Crit. Rev. Biochem., pp. 259-306 (1981).

Removal of carbohydrate moieties present on the ovarian cancer polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of ovarian cancer comprises linking the ovarian cancer polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Ovarian cancer polypeptides of the present invention may also be modified in a way to form chimeric molecules comprising a ovarian cancer polypeptide fused to another, heterologous polypeptide or amino acid sequence. In one embodiment, such a chimeric molecule comprises a fusion of a ovarian cancer polypeptide with a tag polypeptide which provides an epitope to which an anti-tag antibody can selectively bind. The epitope tag is generally placed at the amino-or carboxyl-terminus of the ovarian cancer polypeptide. The presence of such epitope-tagged forms of a ovarian cancer polypeptide can be detected using an antibody against the tag polypeptide. Also, provision of the epitope tag enables the ovarian cancer polypeptide to be readily purified by affinity purification using an anti-tag antibody or another type of affinity matrix that binds to the epitope tag. In an alternative embodiment, the chimeric molecule may comprise a fusion of a ovarian cancer polypeptide with an immunoglobulin or a particular region of an immunoglobulin. For a bivalent form of the chimeric molecule, such a fusion could be to the Fe region of an IgG molecule.

Various tag polypeptides and their respective antibodies are well known in the art. Examples include poly-histidine (poly-his) or poly-histidine-glycine (poly-his-gly) tags; HIS6 and metal chelation tags, the flu HA tag polypeptide and its antibody 12CA5 (Field et al., Mol. Cell. Biol. 8:2159-2165 (1988)); the c-myc tag and the 8F9, 3C7, 6E10, G4, B7 and 9E10 antibodies thereto (Evan et al., Molecular and Cellular Biology 5:3610-3616 (1985)); and the Herpes Simplex virus glycoprotein D (gD) tag and its antibody (Paborsky et al., Protein Engineering 3(6):547-553 (1990)). Other tag polypeptides include the Flag-peptide (Hopp et al., BioTechnology 6:1204-1210 (1988)); the KT3 epitope peptide (Martin et al., Science 255:192-194 (1992)); tubulin epitope peptide (Skinner et al., J. Biol. Chem. 266:15163-15166 (1991)); and the T7 gene 10 protein peptide tag (Lutz-Freyermuth et al., Proc. Natl. Acad. Sci. USA 87:6393-6397 (1990)).

Also included are other ovarian cancer proteins of the ovarian cancer family, and ovarian cancer proteins from other organisms, which are cloned and expressed as outlined below. Thus, probe or degenerate polymerase chain reaction (PCR) primer sequences may be used to find other related ovarian cancer proteins from humans or other organisms. As will be appreciated by those in the art, particularly useful probe and/or PCR primer sequences include the unique areas of the ovarian cancer nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art (e.g., Innis, PCR Protocols, supra).

Antibodies to Ovarian Cancer Proteins

In a preferred embodiment, when the ovarian cancer protein is to be used to generate antibodies, e.g., for immunotherapy or immunodiagnosis, the ovarian cancer protein should share at least one epitope or determinant with the full length protein. By “epitope” or “determinant” herein is typically meant a portion of a protein which will generate and/or bind an antibody or T-cell receptor in the context of MHC. Thus, in most instances, antibodies made to a smaller ovarian cancer protein will be able to bind to the full-length protein, particularly linear epitopes. In a preferred embodiment, the epitope is unique; that is, antibodies generated to a unique epitope show little or no cross-reactivity.

Methods of preparing polyclonal antibodies are known to the skilled artisan (e.g., Coligan, supra; and Harlow & Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler & Milstein, Nature 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1-6 or fragment thereof, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, pp. 59-103 (1986)). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are monoclonal, preferably human or humanized, antibodies that have binding specificities for at least two different antigens or that have binding specificities for two epitopes on the same antigen. In one embodiment, one of the binding specificities is for a protein encoded by a nucleic acid Table 1-6 or a fragment thereof, the other one is for any other antigen, and preferably for a cell-surface protein or receptor or receptor subunit, preferably one that is tumor specific. Alternatively, tetramer-type technology may create multivalent reagents.

In a preferred embodiment, the antibodies to ovarian cancer protein are capable of reducing or eliminating a biological function of a ovarian cancer protein, as is described below. That is, the addition of anti-ovarian cancer protein antibodies (either polyclonal or preferably monoclonal) to ovarian cancer tissue (or cells containing ovarian cancer) may reduce or eliminate the ovarian cancer. Generally, at least a 25% decrease in activity, growth, size or the like is preferred, with at least about 50% being particularly preferred and about a 95-100% decrease being especially preferred.

In a preferred embodiment the antibodies to the ovarian cancer proteins are humanized antibodies (e.g., Xenerex Biosciences, Mederex, Inc., Abgenix, Inc., Protein Design Labs, Inc.) Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

Human antibodies can also be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

By immunotherapy is meant treatment of ovarian cancer with an antibody raised against ovarian cancer proteins. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen, leading to an immune response.

In a preferred embodiment the ovarian cancer proteins against which antibodies are raised are secreted proteins as described above. Without being bound by theory, antibodies used for treatment, bind and prevent the secreted protein from binding to its receptor, thereby inactivating the secreted ovarian cancer protein.

In another preferred embodiment, the ovarian cancer protein to which antibodies are raised is a transmembrane protein. Without being bound by theory, antibodies used for treatment, bind the extracellular domain of the ovarian cancer protein and prevent it from binding to other proteins, such as circulating ligands or cell-associated molecules. The antibody may cause down-regulation of the transmembrane ovarian cancer protein. As will be appreciated by one of ordinary skill in the art, the antibody may be a competitive, non-competitive or uncompetitive inhibitor of protein binding to the extracellular domain of the ovarian cancer protein. The antibody is also an antagonist of the ovarian cancer protein. Further, the antibody prevents activation of the transmembrane ovarian cancer protein. In one aspect, when the antibody prevents the binding of other molecules to the ovarian cancer protein, the antibody prevents growth of the cell. The antibody may also be used to target or sensitize the cell to cytotoxic agents, including, but not limited to TNF-α, TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity or antigen-dependent cytotoxicity (ADCC). Thus, ovarian cancer is treated by administering to a patient antibodies directed against the transmembrane ovarian cancer protein. Antibody-labeling may activate a co-toxin, localize a toxin payload, or otherwise provide means to locally ablate cells.

In another preferred embodiment, the antibody is conjugated to an effector moiety. The effector moiety can be any number of molecules, including labelling moieties such as radioactive labels or fluorescent labels, or can be a therapeutic moiety. In one aspect the therapeutic moiety is a small molecule that modulates the activity of the ovarian cancer protein. In another aspect the therapeutic moiety modulates the activity of molecules associated with or in close proximity to the ovarian cancer protein. The therapeutic moiety may inhibit enzymatic activity such as protease or collagenase or protein kinase activity associated with ovarian cancer.

In a preferred embodiment, the therapeutic moiety can also be a cytotoxic agent. In this method, targeting the cytotoxic agent to ovarian cancer tissue or cells, results in a reduction in the number of afflicted cells, thereby reducing symptoms associated with ovarian cancer. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against ovarian cancer proteins, or binding of a radionuclide to a chelating agent that has been covalently attached to the antibody. Targeting the therapeutic moiety to transmembrane ovarian cancer proteins not only serves to increase the local concentration of therapeutic moiety in the ovarian cancer afflicted area, but also serves to reduce deleterious side effects that may be associated with the therapeutic moiety.

In another preferred embodiment, the ovarian cancer protein against which the antibodies are raised is an intracellular protein. In this case, the antibody may be conjugated to a protein which facilitates entry into the cell. In one case, the antibody enters the cell by endocytosis. In another embodiment, a nucleic acid encoding the antibody is administered to the individual or cell. Moreover, wherein the ovarian cancer protein can be targeted within a cell, i.e., the nucleus, an antibody thereto contains a signal for that target localization, i.e., a nuclear localization signal.

The ovarian cancer antibodies of the invention specifically bind to ovarian cancer proteins. By “specifically bind” herein is meant that the antibodies bind to the protein with a K_{d} of at least about 0.1 mM, more usually at least about 1 μM, preferably at least about 0.1 μM or better, and most preferably, 0.01 μM or better. Selectivity of binding is also important.

Detection of Ovarian Cancer Sequence for Diagnostic and Therapeutic Applications

In one aspect, the RNA expression levels of genes are determined for different cellular states in the ovarian cancer phenotype. Expression levels of genes in normal tissue (i.e., not undergoing ovarian cancer) and in ovarian cancer tissue (and in some cases, for varying severities of ovarian cancer that relate to prognosis, as outlined below) are evaluated to provide expression profiles. An expression profile of a particular cell state or point of development is essentially a “fingerprint” of the state. While two states may have any particular gene similarly expressed, the evaluation of a number of genes simultaneously allows the generation of a gene expression profile that is reflective of the state of the cell. By comparing expression profiles of cells in different states, information regarding which genes are important (including both up- and down-regulation of genes) in each of these states is obtained. Then, diagnosis may be performed or confirmed to determine whether a tissue sample has the gene expression profile of normal or cancerous tissue. This will provide for molecular diagnosis of related conditions.

“Differential expression,” or grammatical equivalents as used herein, refers to qualitative or quantitative differences in the temporal and/or cellular gene expression patterns within and among cells and tissue. Thus, a differentially expressed gene can qualitatively have its expression altered, including an activation or inactivation, in, e.g., normal versus ovarian cancer tissue. Genes may be turned on or turned off in a particular state, relative to another state thus permitting comparison of two or more states. A qualitatively regulated gene will exhibit an expression pattern within a state or cell type which is detectable by standard techniques. Some genes will be expressed in one state or cell type, but not in both. Alternatively, the difference in expression may be quantitative, e.g., in that expression is increased or decreased; i.e., gene expression is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology 14:1675-1680 (1996), hereby expressly incorporated by reference. Other techniques include, but are not limited to, quantitative reverse transcriptase PCR, northern analysis and RNase protection. As outlined above, preferably the change in expression (i.e., upregulation or downregulation) is at least about 50%, more preferably at least about 100%, more preferably at least about 150%, more preferably at least about 200%, with from 300 to at least 1000% being especially preferred.

Evaluation may be at the gene transcript, or the protein level. The amount of gene expression may be monitored using nucleic acid probes to the DNA or RNA equivalent of the gene transcript, and the quantification of gene expression levels, or, alternatively, the final gene product itself (protein) can be monitored, e.g., with antibodies to the ovarian cancer protein and standard immunoassays (ELISAs, etc.) or other techniques, including mass spectroscopy assays, 2D gel electrophoresis assays, etc. Proteins corresponding to ovarian cancer genes, i.e., those identified as being important in a ovarian cancer phenotype, can be evaluated in a ovarian cancer diagnostic test.

In a preferred embodiment, gene expression monitoring is performed simultaneously on a number of genes. Multiple protein expression monitoring can be performed as well. Similarly, these assays may be performed on an individual basis as well.

In this embodiment, the ovarian cancer nucleic acid probes are attached to biochips as outlined herein for the detection and quantification of ovarian cancer sequences in a particular cell. The assays are further described below in the example. PCR techniques can be used to provide greater sensitivity.

In a preferred embodiment nucleic acids encoding the ovarian cancer protein are detected. Although DNA or RNA encoding the ovarian cancer protein may be detected, of particular interest are methods wherein an mRNA encoding a ovarian cancer protein is detected. Probes to detect mRNA can be a nucleotide/deoxynucleotide probe that is complementary to and hybridizes with the mRNA and includes, but is not limited to, oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a ovarian cancer protein is detected by binding the digoxygenin with an anti-digoxygenin secondary antibody and developed with nitro blue tetrazolium and 5-bromo-4-chloro-3-indoyl phosphate.

In a preferred embodiment, various proteins from the three classes of proteins as described herein (secreted, transmembrane or intracellular proteins) are used in diagnostic assays. The ovarian cancer proteins, antibodies, nucleic acids, modified proteins and cells containing ovarian cancer sequences are used in diagnostic assays. This can be performed on an individual gene or corresponding polypeptide level. In a preferred embodiment, the expression profiles are used, preferably in conjunction with high throughput screening techniques to allow monitoring for expression profile genes and/or corresponding polypeptides.

As described and defined herein, ovarian cancer proteins, including intracellular, transmembrane or secreted proteins, find use as markers of ovarian cancer. Detection of these proteins in putative ovarian cancer tissue allows for detection or diagnosis of ovarian cancer. In one embodiment, antibodies are used to detect ovarian cancer proteins. A preferred method separates proteins from a sample by electrophoresis on a gel (typically a denaturing and reducing protein gel, but may be another type of gel, including isoelectric focusing gels and the like). Following separation of proteins, the ovarian cancer protein is detected, e.g., by immunoblotting with antibodies raised against the ovarian cancer protein. Methods of immunoblotting are well known to those of ordinary skill in the art.

In another preferred method, antibodies to the ovarian cancer protein find use in in situ imaging techniques, e.g., in histology (e.g., Methods in Cell Biology: Antibodies in Cell Biology, volume 37 (Asai, ed. 1993)). In this method cells are contacted with from one to many antibodies to the ovarian cancer protein(s). Following washing to remove non-specific antibody binding, the presence of the antibody or antibodies is detected. In one embodiment the antibody is detected by incubating with a secondary antibody that contains a detectable label. In another method the primary antibody to the ovarian cancer protein(s) contains a detectable label, e.g. an enzyme marker that can act on a substrate. In another preferred embodiment each one of multiple primary antibodies contains a distinct and detectable label. This method finds particular use in simultaneous screening for a plurality of ovarian cancer proteins. As will be appreciated by one of ordinary skill in the art, many other histological imaging techniques are also provided by the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing ovarian cancer from blood, serum, plasma, stool, and other samples. Such samples, therefore, are useful as samples to be probed or tested for the presence of ovarian cancer proteins. Antibodies can be used to detect a ovarian cancer protein by previously described immunoassay techniques including ELISA, immunoblotting (western blotting), immunoprecipitation, BIACORE technology and the like. Conversely, the presence of antibodies may indicate an immune response against an endogenous ovarian cancer protein.

In a preferred embodiment, in situ hybridization of labeled ovarian cancer nucleic acid probes to tissue arrays is done. For example, arrays of tissue samples, including ovarian cancer tissue and/or normal tissue, are made. In situ hybridization (see, e.g., Ausubel, supra) is then performed. When comparing the fingerprints between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or may be predictive of outcomes.

In a preferred embodiment, the ovarian cancer proteins, antibodies, nucleic acids, modified proteins and cells containing ovarian cancer sequences are used in prognosis assays. As above, gene expression profiles can be generated that correlate to ovarian cancer, in terms of long term prognosis. Again, this may be done on either a protein or gene level, with the use of genes being preferred. As above, ovarian cancer probes may be attached to biochips for the detection and quantification of ovarian cancer sequences in a tissue or patient. The assays proceed as outlined above for diagnosis. PCR method may provide more sensitive and accurate quantification.

Assays for Therapeutic Compounds

In a preferred embodiment members of the proteins, nucleic acids, and antibodies as described herein are used in drug screening assays. The ovarian cancer proteins, antibodies, nucleic acids, modified proteins and cells containing ovarian cancer sequences are used in drug screening assays or by evaluating the effect of drug candidates on a “gene expression profile” or expression profile of polypeptides. In a preferred embodiment, the expression profiles are used, preferably in conjunction with high throughput screening techniques to allow monitoring for expression profile genes after treatment with a candidate agent (e.g., Zlokarnik, et al., Science 279:84-8 (1998); Heid, Genome Res 6:986-94, 1996).

In a preferred embodiment, the ovarian cancer proteins, antibodies, nucleic acids, modified proteins and cells containing the native or modified ovarian cancer proteins are used in screening assays. That is, the present invention provides novel methods for screening for compositions which modulate the ovarian cancer phenotype or an identified physiological function of a ovarian cancer protein. As above, this can be done on an individual gene level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the differentially expressed genes herein, a variety of assays may be executed. In a preferred embodiment, assays may be run on an individual gene or protein level. That is, having identified a particular gene as up regulated in ovarian cancer, test compounds can be screened for the ability to modulate gene expression or for binding to the ovarian cancer protein. “Modulation” thus includes both an increase and a decrease in gene expression. The preferred amount of modulation will depend on the original change of the gene expression in normal versus tissue undergoing ovarian cancer, with changes of at least 10%, preferably 50%, more preferably 100-300%, and in some embodiments 300-1000% or greater. Thus, if a gene exhibits a 4-fold increase in ovarian cancer tissue compared to normal tissue, a decrease of about four-fold is often desired; similarly, a 10-fold decrease in ovarian cancer tissue compared to normal tissue often provides a target value of a 10-fold increase in expression to be induced by the test compound.

The amount of gene expression may be monitored using nucleic acid probes and the quantification of gene expression levels, or, alternatively, the gene product itself can be monitored, e.g., through the use of antibodies to the ovarian cancer protein and standard immunoassays. Proteomics and separation techniques may also allow quantification of expression.

In a preferred embodiment, gene expression or protein monitoring of a number of entities, i.e., an expression profile, is monitored simultaneously. Such profiles will typically involve a plurality of those entities described herein.

In this embodiment, the ovarian cancer nucleic acid probes are attached to biochips as outlined herein for the detection and quantification of ovarian cancer sequences in a particular cell. Alternatively, PCR may be used. Thus, a series, e.g., of microtiter plate, may be used with dispensed primers in desired wells. A PCR reaction can then be performed and analyzed for each well.

Expression monitoring can be performed to identify compounds that modify the expression of one or more ovarian cancer-associated sequences, e.g., a polynucleotide sequence set out in Tables 1-6. Generally, in a preferred embodiment, a test modulator is added to the cells prior to analysis. Moreover, screens are also provided to identify agents that modulate ovarian cancer, modulate ovarian cancer proteins, bind to a ovarian cancer protein, or interfere with the binding of a ovarian cancer protein and an antibody or other binding partner.

The term “test compound” or “drug candidate” or “modulator” or grammatical equivalents as used herein describes any molecule, e.g., protein, oligopeptide, small organic molecule, polysaccharide, polynucleotide, etc., to be tested for the capacity to directly or indirectly alter the ovarian cancer phenotype or the expression of a ovarian cancer sequence, e.g., a nucleic acid or protein sequence. In preferred embodiments, modulators alter expression profiles, or expression profile nucleic acids or proteins provided herein. In one embodiment, the modulator suppresses a ovarian cancer phenotype, e.g. to a normal tissue fingerprint. In another embodiment, a modulator induced a ovarian cancer phenotype. Generally, a plurality of assay mixtures are run in parallel with different agent concentrations to obtain a differential response to the various concentrations. Typically, one of these concentrations serves as a negative control, i.e., at zero concentration or below the level of detection.

Drug candidates encompass numerous chemical classes, though typically they are organic molecules, preferably small organic compounds having a molecular weight of more than 100 and less than about 2,500 daltons. Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

In one aspect, a modulator will neutralize the effect of a ovarian cancer protein. By “neutralize” is meant that activity of a protein is inhibited or blocked and the consequent effect on the cell.

In certain embodiments, combinatorial libraries of potential modulators will be screened for an ability to bind to a ovarian cancer polypeptide or to modulate activity. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al., J. Med. Chem. 37(9):1233-1251 (1994)).

Preparation and screening of combinatorial chemical libraries is well known to those of skill in the art. Such combinatorial chemical libraries include, but are not limited to, peptide libraries (see, e.g., U.S. Pat. No. 5,010,175, Furka, Pept. Prot. Res. 37:487-493 (1991), Houghton et al., Nature, 354:84-88 (1991)), peptoids (PCT Publication No WO 91/19735), encoded peptides (PCT Publication WO 93/20242), random bio-oligomers (PCT Publication WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with a Beta-D-Glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho, et al., Science 261:1303 (1993)), and/or peptidyl phosphonates (Campbell et al., J. Org. Chem. 59:658 (1994)). See, generally, Gordon et al., J. Med. Chem. 37:1385 (1994), nucleic acid libraries (see, e.g., Strategene, Corp.), peptide nucleic acid libraries (see, e.g., U.S. Pat. No. 5,539,083), antibody libraries (see, e.g., Vaughn et al., Nature Biotechnology 14(3):309-314 (1996), and PCT/US96/10287), carbohydrate libraries (see, e.g., Liang et al., Science 274:1520-1522 (1996), and U.S. Pat. No. 5,593,853), and small organic molecule libraries (see, e.g., benzodiazepines, Baum, C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514; and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

The assays to identify modulators are amenable to high throughput screening. Preferred assays thus detect enhancement or inhibition of ovarian cancer gene transcription, inhibition or enhancement of polypeptide expression, and inhibition or enhancement of polypeptide activity.

High throughput assays for the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures, including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

In one embodiment, modulators are proteins, often naturally occurring proteins or fragments of naturally occurring proteins. Thus, e.g., cellular extracts containing proteins, or random or directed digests of proteinaceous cellular extracts, may be used. In this way libraries of proteins may be made for screening in the methods of the invention. Particularly preferred in this embodiment are libraries of bacterial, fungal, viral, and mammalian proteins, with the latter being preferred, and human proteins being especially preferred. Particularly useful test compound will be directed to the class of proteins to which the target belongs, e.g., substrates for enzymes or ligands and receptors.

In a preferred embodiment, modulators are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, e.g., of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

Modulators of ovarian cancer can also be nucleic acids, as defined above.

As described above generally for proteins, nucleic acid modulating agents may be naturally occurring nucleic acids, random nucleic acids, or “biased” random nucleic acids. For example, digests of procaryotic or eucaryotic genomes may be used as is outlined above for proteins.

In a preferred embodiment, the candidate compounds are organic chemical moieties, a wide variety of which are available in the literature.

After the candidate agent has been added and the cells allowed to incubate for some period of time, the sample containing a target sequence to be analyzed is added to the biochip. If required, the target sequence is prepared using known techniques. For example, the sample may be treated to lyse the cells, using known lysis buffers, electroporation, etc., with purification and/or amplification such as PCR performed as appropriate. For example, an in vitro transcription with labels covalently attached to the nucleotides is performed. Generally, the nucleic acids are labeled with biotin-FITC or PE, or with cy3 or cy5.

In a preferred embodiment, the target sequence is labeled with, e.g., a fluorescent, a chemiluminescent, a chemical, or a radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. Unbound labeled streptavidin is typically removed prior to analysis.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways. Components of the reaction may be added simultaneously, or sequentially, in different orders, with preferred embodiments outlined below. In addition, the reaction may include a variety of other reagents. These include salts, buffers, neutral proteins, e.g. albumin, detergents, etc. which may be used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

The assay data are analyzed to determine the expression levels, and changes in expression levels as between states, of individual genes, forming a gene expression profile.

Screens are performed to identify modulators of the ovarian cancer phenotype. In one embodiment, screening is performed to identify modulators that can induce or suppress a particular expression profile, thus preferably generating the associated phenotype. In another embodiment, e.g., for diagnostic applications, having identified differentially expressed genes important in a particular state, screens can be performed to identify modulators that alter expression of individual genes. In an another embodiment, screening is performed to identify modulators that alter a biological function of the expression product of a differentially expressed gene. Again, having identified the importance of a gene in a particular state, screens are performed to identify agents that bind and/or modulate the biological activity of the gene product.

In addition screens can be done for genes that are induced in response to a candidate agent. After identifying a modulator based upon its ability to suppress a ovarian cancer expression pattern leading to a normal expression pattern, or to modulate a single ovarian cancer gene expression profile so as to mimic the expression of the gene from normal tissue, a screen as described above can be performed to identify genes that are specifically modulated in response to the agent. Comparing expression profiles between normal tissue and agent treated ovarian cancer tissue reveals genes that are not expressed in normal tissue or ovarian cancer tissue, but are expressed in agent treated tissue. These agent-specific sequences can be identified and used by methods described herein for ovarian cancer genes or proteins. In particular these sequences and the proteins they encode find use in marking or identifying agent treated cells. In addition, antibodies can be raised against the agent induced proteins and used to target novel therapeutics to the treated ovarian cancer tissue sample.

Thus, in one embodiment, a test compound is administered to a population of ovarian cancer cells, that have an associated ovarian cancer expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) may be put into a viral construct such as an adenoviral or retroviral construct, and added to the cell, such that expression of the peptide agent is accomplished, e.g., PCT US97/01019. Regulatable gene therapy systems can also be used.

Once the test compound has been administered to the cells, the cells can be washed if desired and are allowed to incubate under preferably physiological conditions for some period of time. The cells are then harvested and a new gene expression profile is generated, as outlined herein.

Thus, e.g., ovarian cancer tissue may be screened for agents that modulate, e.g., induce or suppress the ovarian cancer phenotype. A change in at least one gene, preferably many, of the expression profile indicates that the agent has an effect on ovarian cancer activity. By defining such a signature for the ovarian cancer phenotype, screens for new drugs that alter the phenotype can be devised. With this approach, the drug target need not be known and need not be represented in the original expression screening platform, nor does the level of transcript for the target protein need to change.

In a preferred embodiment, as outlined above, screens may be done on individual genes and gene products (proteins). That is, having identified a particular differentially expressed gene as important in a particular state, screening of modulators of either the expression of the gene or the gene product itself can be done. The gene products of differentially expressed genes are sometimes referred to herein as “ovarian cancer proteins” or a “ovarian cancer modulatory protein”. The ovarian cancer modulatory protein may be a fragment, or alternatively, be the full length protein to the fragment encoded by the nucleic acids of the Tables. Preferably, the ovarian cancer modulatory protein is a fragment. In a preferred embodiment, the ovarian cancer amino acid sequence which is used to determine sequence identity or similarity is encoded by a nucleic acid of the Tables. In another embodiment, the sequences are naturally occurring allelic variants of a protein encoded by a nucleic acid of the Tables. In another embodiment, the sequences are sequence variants as further described herein.

Preferably, the ovarian cancer modulatory protein is a fragment of approximately 14 to 24 amino acids long. More preferably the fragment is a soluble fragment. Preferably, the fragment includes a non-transmembrane region. In a preferred embodiment, the fragment has an N-terminal Cys to aid in solubility. In one embodiment, the C-terminus of the fragment is kept as a free acid and the N-terminus is a free amine to aid in coupling, i.e., to cysteine.

In one embodiment the ovarian cancer proteins are conjugated to an immunogenic agent as discussed herein. In one embodiment the ovarian cancer protein is conjugated to BSA.

Measurements of ovarian cancer polypeptide activity, or of ovarian cancer or the ovarian cancer phenotype can be performed using a variety of assays. For example, the effects of the test compounds upon the function of the ovarian cancer polypeptides can be measured by examining parameters described above. A suitable physiological change that affects activity can be used to assess the influence of a test compound on the polypeptides of this invention. When the functional consequences are determined using intact cells or animals, one can also measure a variety of effects such as, in the case of ovarian cancer associated with tumors, tumor growth, tumor metastasis, neovascularization, hormone release, transcriptional changes to both known and uncharacterized genetic markers (e.g., northern blots), changes in cell metabolism such as cell growth or pH changes, and changes in intracellular second messengers such as cGMP. In the assays of the invention, mammalian ovarian cancer polypeptide is typically used, e.g., mouse, preferably human.

Assays to identify compounds with modulating activity can be performed in vitro. For example, a ovarian cancer polypeptide is first contacted with a potential modulator and incubated for a suitable amount of time, e.g., from 0.5 to 48 hours. In one embodiment, the ovarian cancer polypeptide levels are determined in vitro by measuring the level of protein or mRNA. The level of protein is measured using immunoassays such as western blotting, ELISA and the like with an antibody that selectively binds to the ovarian cancer polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, a reporter gene system can be devised using the ovarian cancer protein promoter operably linked to a reporter gene such as luciferase, green fluorescent protein, CAT, or β-gal. The reporter construct is typically transfected into a cell. After treatment with a potential modulator, the amount of reporter gene transcription, translation, or activity is measured according to standard techniques known to those of skill in the art.

In a preferred embodiment, as outlined above, screens may be done on individual genes and gene products (proteins). That is, having identified a particular differentially expressed gene as important in a particular state, screening of modulators of the expression of the gene or the gene product itself can be done. The gene products of differentially expressed genes are sometimes referred to herein as “ovarian cancer proteins.” The ovarian cancer protein may be a fragment, or alternatively, be the full length protein to a fragment shown herein.

In one embodiment, screening for modulators of expression of specific genes is performed. Typically, the expression of only one or a few genes are evaluated. In another embodiment, screens are designed to first find compounds that bind to differentially expressed proteins. These compounds are then evaluated for the ability to modulate differentially expressed activity. Moreover, once initial candidate compounds are identified, variants can be further screened to better evaluate structure activity relationships.

In a preferred embodiment, binding assays are done. In general, purified or isolated gene product is used; that is, the gene products of one or more differentially expressed nucleic acids are made. For example, antibodies are generated to the protein gene products, and standard immunoassays are run to determine the amount of protein present. Alternatively, cells comprising the ovarian cancer proteins can be used in the assays.

Thus, in a preferred embodiment, the methods comprise combining a ovarian cancer protein and a candidate compound, and determining the binding of the compound to the ovarian cancer protein. Preferred embodiments utilize the human ovarian cancer protein, although other mammalian proteins may also be used, e.g. for the development of animal models of human disease. In some embodiments, as outlined herein, variant or derivative ovarian cancer proteins may be used.

Generally, in a preferred embodiment of the methods herein, the ovarian cancer protein or the candidate agent is non-diffusably bound to an insoluble support having isolated sample receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the ovarian cancer protein is bound to the support, and a test compound is added to the assay. Alternatively, the candidate agent is bound to the support and the ovarian cancer protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the test modulating compound to the ovarian cancer protein may be done in a number of ways. In a preferred embodiment, the compound is labeled, and binding determined directly, e.g., by attaching all or a portion of the ovarian cancer protein to a solid support, adding a labeled candidate agent (e.g., a fluorescent label), washing off excess reagent, and determining whether the label is present on the solid support. Various blocking and washing steps may be utilized as appropriate.

In some embodiments, only one of the components is labeled, e.g., the proteins (or proteinaceous candidate compounds) can be labeled. Alternatively, more than one component can be labeled with different labels, e.g., ¹²⁵I for the proteins and a fluorophor for the compound. Proximity reagents, e.g., quenching or energy transfer reagents are also useful.

In one embodiment, the binding of the test compound is determined by competitive binding assay. The competitor is a binding moiety known to bind to the target molecule (i.e., a ovarian cancer protein), such as an antibody, peptide, binding partner, ligand, etc. Under certain circumstances, there may be competitive binding between the compound and the binding moiety, with the binding moiety displacing the compound. In one embodiment, the test compound is labeled. Either the compound, or the competitor, or both, is added first to the protein for a time sufficient to allow binding, if present. Incubations may be performed at a temperature which facilitates optimal activity, typically between 4 and 40° C. Incubation periods are typically optimized, e.g., to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the test compound. Displacement of the competitor is an indication that the test compound is binding to the ovarian cancer protein and thus is capable of binding to, and potentially modulating, the activity of the ovarian cancer protein. In this embodiment, either component can be labeled. Thus, e.g., if the competitor is labeled, the presence of label in the wash solution indicates displacement by the agent. Alternatively, if the test compound is labeled, the presence of the label on the support indicates displacement.

In an alternative embodiment, the test compound is added first, with incubation and washing, followed by the competitor. The absence of binding by the competitor may indicate that the test compound is bound to the ovarian cancer protein with a higher affinity. Thus, if the test compound is labeled, the presence of the label on the support, coupled with a lack of competitor binding, may indicate that the test compound is capable of binding to the ovarian cancer protein.

In a preferred embodiment, the methods comprise differential screening to identity agents that are capable of modulating the activity of the ovarian cancer proteins. In this embodiment, the methods comprise combining a ovarian cancer protein and a competitor in a first sample. A second sample comprises a test compound, a ovarian cancer protein, and a competitor. The binding of the competitor is determined for both samples, and a change, or difference in binding between the two samples indicates the presence of an agent capable of binding to the ovarian cancer protein and potentially modulating its activity. That is, if the binding of the competitor is different in the second sample relative to the first sample, the agent is capable of binding to the ovarian cancer protein.

Alternatively, differential screening is used to identify drug candidates that bind to the native ovarian cancer protein, but cannot bind to modified ovarian cancer proteins. The structure of the ovarian cancer protein may be modeled, and used in rational drug design to synthesize agents that interact with that site. Drug candidates that affect the activity of a ovarian cancer protein are also identified by screening drugs for the ability to either enhance or reduce the activity of the protein.

Positive controls and negative controls may be used in the assays. Preferably control and test samples are performed in at least triplicate to obtain statistically significant results. Incubation of all samples is for a time sufficient for the binding of the agent to the protein. Following incubation, samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc. which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in an order that provides for the requisite binding.

In a preferred embodiment, the invention provides methods for screening for a compound capable of modulating the activity of a ovarian cancer protein. The methods comprise adding a test compound, as defined above, to a cell comprising ovarian cancer proteins. Preferred cell types include almost any cell. The cells contain a recombinant nucleic acid that encodes a ovarian cancer protein. In a preferred embodiment, a library of candidate agents are tested on a plurality of cells.

In one aspect, the assays are evaluated in the presence or absence or previous or subsequent exposure of physiological signals, e.g. hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, compounds that modulate ovarian cancer agents are identified. Compounds with pharmacological activity are able to enhance or interfere with the activity of the ovarian cancer protein. Once identified, similar structures are evaluated to identify critical structural feature of the compound.

In one embodiment, a method of inhibiting ovarian cancer cell division is provided. The method comprises administration of a ovarian cancer inhibitor. In another embodiment, a method of inhibiting ovarian cancer is provided. The method comprises administration of a ovarian cancer inhibitor. In a further embodiment, methods of treating cells or individuals with ovarian cancer are provided. The method comprises administration of a ovarian cancer inhibitor.

In one embodiment, a ovarian cancer inhibitor is an antibody as discussed above. In another embodiment, the ovarian cancer inhibitor is an antisense molecule.

A variety of cell growth, proliferation, and metastasis assays are known to those of skill in the art, as described below.

Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate normal phenotype and require a solid substrate to attach and grow. Soft agar growth or colony formation in suspension assays can be used to identify modulators of ovarian cancer sequences, which when expressed in host cells, inhibit abnormal cellular proliferation and transformation. A therapeutic compound would reduce or eliminate the host cells' ability to grow in stirred suspension culture or suspended in semi-solid media, such as semi-solid or soft.

Techniques for soft agar growth or colony formation in suspension assays are described in Freshney, Culture of Animal Cells a Manual of Basic Technique (3^{rd} ed., 1994), herein incorporated by reference. See also, the methods section of Garkavtsev et al. (1996), supra, herein incorporated by reference.

Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This can be detected morphologically by the formation of a disoriented monolayer of cells or rounded cells in foci within the regular pattern of normal surrounding cells. Alternatively, labeling index with (³H)-thymidine at saturation density can be used to measure density limitation of growth. See Freshney (1994), supra. The transformed cells, when transfected with tumor suppressor genes, regenerate a normal phenotype and become contact inhibited and would grow to a lower density.

In this assay, labeling index with (³H)-thymidine at saturation density is a preferred method of measuring density limitation of growth. Transformed host cells are transfected with a ovarian cancer-associated sequence and are grown for 24 hours at saturation density in non-limiting medium conditions. The percentage of cells labeling with (³H)-thymidine is determined autoradiographically. See, Freshney (1994), supra.

Growth Factor or Serum Dependence

Transformed cells have a lower serum dependence than their normal counterparts (see, e.g., Temin, J. Natl. Cancer Insti. 37:167-175 (1966); Eagle et al., J. Exp. Med. 131:836-879 (1970)); Freshney, supra. This is in part due to release of various growth factors by the transformed cells. Growth factor or serum dependence of transformed host cells can be compared with that of control.

Tumor Specific Markers Levels

Tumor cells release an increased amount of certain factors (hereinafter “tumor specific markers”) than their normal counterparts. For example, plasminogen activator (PA) is released from human glioma at a higher level than from normal brain cells (see, e.g., Gullino, Angiogenesis, tumor vascularization, and potential interference with tumor growth. in Biological Responses in Cancer, pp. 178-184 (Mihich (ed.) 1985)). Similarly, Tumor angiogenesis factor (TAF) is released at a higher level in tumor cells than their normal counterparts. See, e.g., Folkman, Angiogenesis and Cancer, Sem Cancer Biol. (1992)).

Various techniques which measure the release of these factors are described in Freshney (1994), supra. Also, see, Unkless et al. , J. Biol. Chem. 249:4295-4305 (1974); Strickland & Beers, J. Biol. Chem. 251:5694-5702 (1976); Whur et al., Br. J. Cancer 42:305-312 (1980); Gullino, Angiogenesis, tumor vascularization, and potential interference with tumor growth. in Biological Responses in Cancer, pp. 178-184 (Mihich (ed.) 1985); Freshney Anticancer Res. 5:111-130 (1985).

Invasiveness into Matrigel

The degree of invasiveness into Matrigel or some other extracellular matrix constituent can be used as an assay to identify compounds that modulate ovarian cancer-associated sequences. Tumor cells exhibit a good correlation between malignancy and invasiveness of cells into Matrigel or some other extracellular matrix constituent. In this assay, tumorigenic cells are typically used as host cells. Expression of a tumor suppressor gene in these host cells would decrease invasiveness of the host cells.

Techniques described in Freshney (1994), supra, can be used. Briefly, the level of invasion of host cells can be measured by using filters coated with Matrigel or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish. See, e.g., Freshney (1984), supra.

Tumor Growth in vivo

Effects of ovarian cancer-associated sequences on cell growth can be tested in transgenic or immune-suppressed mice. Knock-out transgenic mice can be made, in which the ovarian cancer gene is disrupted or in which a ovarian cancer gene is inserted. Knock-out transgenic mice can be made by insertion of a marker gene or other heterologous gene into the endogenous ovarian cancer gene site in the mouse genome via homologous recombination. Such mice can also be made by substituting the endogenous ovarian cancer gene with a mutated version of the ovarian cancer gene, or by mutating the endogenous ovarian cancer gene, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

Alternatively, various immune-suppressed or immune-deficient host animals can be used. For example, genetically athymic “nude” mouse (see, e.g., Giovanella et al., J. Natl. Cancer Inst. 52:921 (1974)), a SCID mouse, a thymectomized mouse, or an irradiated mouse (see, e.g., Bradley et al., Br. J. Cancer 38:263 (1978); Selby et al., Br. J. Cancer 41:52 (1980)) can be used as a host. Transplantable tumor cells (typically about 10⁶ cells) injected into isogenic hosts will produce invasive tumors in a high proportions of cases, while normal cells of similar origin will not. In hosts which developed invasive tumors, cells expressing a ovarian cancer-associated sequences are injected subcutaneously. After a suitable length of time, preferably 4-8 weeks, tumor growth is measured (e.g., by volume or by its two largest dimensions) and compared to the control. Tumors that have statistically significant reduction (using, e.g., Student's T test) are said to have inhibited growth.

Polynucleotide Modulators of Ovarian Cancer

Antisense Polynucleotides

In certain embodiments, the activity of a ovarian cancer-associated protein is down-regulated, or entirely inhibited, by the use of antisense polynucleotide, i.e., a nucleic acid complementary to, and which can preferably hybridize specifically to, a coding mRNA nucleic acid sequence, e.g., a ovarian cancer protein mRNA, or a subsequence thereof. Binding of the antisense polynucleotide to the mRNA reduces the translation and/or stability of the mRNA.

In the context of this invention, antisense polynucleotides can comprise naturally-occurring nucleotides, or synthetic species formed from naturally-occurring subunits or their close homologs. Antisense polynucleotides may also have altered sugar moieties or inter-sugar linkages. Exemplary among these are the phosphorothioate and other sulfur containing species which are known for use in the art. Analogs are comprehended by this invention so long as they function effectively to hybridize with the ovarian cancer protein mRNA. See, e.g., Isis Pharmaceuticals, Carlsbad, Calif.; Sequitor, Inc., Natick, Mass.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

Antisense molecules as used herein include antisense or sense oligonucleotides. Sense oligonucleotides can, e.g., be employed to block transcription by binding to the anti-sense strand. The antisense and sense oligonucleotide comprise a single-stranded nucleic acid sequence (either RNA or DNA) capable of binding to target mRNA (sense) or DNA (antisense) sequences for ovarian cancer molecules. A preferred antisense molecule is for a ovarian cancer sequences in Tables 1-6, or for a ligand or activator thereof. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, e.g., Stein & Cohen (Cancer Res. 48:2659 (1988 and van der Krol et al. (BioTechniques 6:958 (1988)).

Ribozymes

In addition to antisense polynucleotides, ribozymes can be used to target and inhibit transcription of ovarian cancer-associated nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes (see, e.g., Castanotto et al., Adv. in Pharmacology 25: 289-317 (1994) for a general review of the properties of different ribozymes).

The general features of hairpin ribozymes are described, e.g., in Hampel et al., Nucl. Acids Res. 18:299-304 (1990); European Patent Publication No. 0 360 257; U.S. Pat. No. 5,254,678. Methods of preparing are well known to those of skill in the art (see, e.g., WO 94/26877; Ojwang et al., Proc. Natl. Acad. Sci. USA 90:6340-6344 (1993); Yamada et al., Human Gene Therapy 1:39-45 (1994); Leavitt et al., Proc. Natl. Acad. Sci. USA 92:699-703 (1995); Leavitt et al., Human Gene Therapy 5:1151-120 (1994); and Yamada et al., Virology 205: 121-126 (1994)).

Polynucleotide modulators of ovarian cancer may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a polynucleotide modulator of ovarian cancer may be introduced into a cell containing the target nucleic acid sequence, e.g., by formation of an polynucleotide-lipid complex, as described in WO 90/10448. It is understood that the use of antisense molecules or knock out and knock in models may also be used in screening assays as discussed above, in addition to methods of treatment.

Thus, in one embodiment, methods of modulating ovarian cancer in cells or organisms are provided. In one embodiment, the methods comprise administering to a cell an anti-ovarian cancer antibody that reduces or eliminates the biological activity of an endogenous ovarian cancer protein. Alternatively, the methods comprise administering to a cell or organism a recombinant nucleic acid encoding a ovarian cancer protein. This may be accomplished in any number of ways. In a preferred embodiment, e.g. when the ovarian cancer sequence is down-regulated in ovarian cancer, such state may be reversed by increasing the amount of ovarian cancer gene product in the cell. This can be accomplished, e.g., by overexpressing the endogenous ovarian cancer gene or administering a gene encoding the ovarian cancer sequence, using known gene-therapy techniques, e.g. In a preferred embodiment, the gene therapy techniques include the incorporation of the exogenous gene using enhanced homologous recombination (EHR), e.g. as described in PCT/US93/03868, hereby incorporated by reference in its entirety. Alternatively, e.g. when the ovarian cancer sequence is up-regulated in ovarian cancer, the activity of the endogenous ovarian cancer gene is decreased, e.g. by the administration of a ovarian cancer antisense nucleic acid.

In one embodiment, the ovarian cancer proteins of the present invention may be used to generate polyclonal and monoclonal antibodies to ovarian cancer proteins. Similarly, the ovarian cancer proteins can be coupled, using standard technology, to affinity chromatography columns. These columns may then be used to purify ovarian cancer antibodies useful for production, diagnostic, or therapeutic purposes. In a preferred embodiment, the antibodies are generated to epitopes unique to a ovarian cancer protein; that is, the antibodies show little or no cross-reactivity to other proteins. The ovarian cancer antibodies may be coupled to standard affinity chromatography columns and used to purify ovarian cancer proteins. The antibodies may also be used as blocking polypeptides, as outlined above, since they will specifically bind to the ovarian cancer protein.

Methods of Identifying Variant Ovarian Cancer-Associated Sequences

Without being bound by theory, expression of various ovarian cancer sequences is correlated with ovarian cancer. Accordingly, disorders based on mutant or variant ovarian cancer genes may be determined. In one embodiment, the invention provides methods for identifying cells containing variant ovarian cancer genes, e.g., determining all or part of the sequence of at least one endogenous ovarian cancer genes in a cell. This may be accomplished using any number of sequencing techniques. In a preferred embodiment, the invention provides methods of identifying the ovarian cancer genotype of an individual, e.g., determining all or part of the sequence of at least one ovarian cancer gene of the individual. This is generally done in at least one tissue of the individual, and may include the evaluation of a number of tissues or different samples of the same tissue. The method may include comparing the sequence of the sequenced ovarian cancer gene to a known ovarian cancer gene, i.e., a wild-type gene.

The sequence of all or part of the ovarian cancer gene can then be compared to the sequence of a known ovarian cancer gene to determine if any differences exist. This can be done using any number of known homology programs, such as Bestfit, etc. In a preferred embodiment, the presence of a difference in the sequence between the ovarian cancer gene of the patient and the known ovarian cancer gene correlates with a disease state or a propensity for a disease state, as outlined herein.

In a preferred embodiment, the ovarian cancer genes are used as probes to determine the number of copies of the ovarian cancer gene in the genome.

In another preferred embodiment, the ovarian cancer genes are used as probes to determine the chromosomal localization of the ovarian cancer genes. Information such as chromosomal localization finds use in providing a diagnosis or prognosis in particular when chromosomal abnormalities such as translocations, and the like are identified in the ovarian cancer gene locus.

Administration of Pharmaceutical and Vaccine Compositions

In one embodiment, a therapeutically effective dose of a ovarian cancer protein or modulator thereof, is administered to a patient. By “therapeutically effective dose” herein is meant a dose that produces effects for which it is administered. The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)). As is known in the art, adjustments for ovarian cancer degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art. U.S. patent application Ser. No. 09/687,576, further discloses the use of compositions and methods of diagnosis and treatment in ovarian cancer is hereby expressly incorporated by reference.

A “patient” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

The administration of the ovarian cancer proteins and modulators thereof of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, e.g., in the treatment of wounds and inflammation, the ovarian cancer proteins and modulators may be directly applied as a solution or spray.

The pharmaceutical compositions of the present invention comprise a ovarian cancer protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that ovarian cancer protein modulators (e.g., antibodies, antisense constructs, ribozymes, small organic molecules, etc.) when administered orally, should be protected from digestion. This is typically accomplished either by complexing the molecule(s) with a composition to render it resistant to acidic and enzymatic hydrolysis, or by packaging the molecule(s) in an appropriately resistant carrier, such as a liposome or a protection barrier. Means of protecting agents from digestion are well known in the art.

The compositions for administration will commonly comprise a ovarian cancer protein modulator dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacologial Basis of Therapeutics (Hardman et al., eds., 1996)).

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art, e.g., Remington's Pharmaceutical Science and Goodman and Gillman, The Pharmacologial Basis of Therapeutics, supra.

The compositions containing modulators of ovarian cancer proteins can be administered for therapeutic or prophylactic treatments. In therapeutic applications, compositions are administered to a patient suffering from a disease (e.g., a cancer) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient. An amount of modulator that is capable of preventing or slowing the development of cancer in a mammal is referred to as a “prophylactically effective dose.” The particular dose required for a prophylactic treatment will depend upon the medical condition and history of the mammal, the particular cancer being prevented, as well as other factors such as age, weight, gender, administration route, efficiency, etc. Such prophylactic treatments may be used, e.g., in a mammal who has previously had cancer to prevent a recurrence of the cancer, or in a mammal who is suspected of having a significant likelihood of developing cancer.

It will be appreciated that the present ovarian cancer protein-modulating compounds can be administered alone or in combination with additional ovarian cancer modulating compounds or with other therapeutic agent, e.g., other anti-cancer agents or treatments.

In numerous embodiments, one or more nucleic acids, e.g., polynucleotides comprising nucleic acid sequences set forth in Tables 1-6, such as antisense polynucleotides or ribozymes, will be introduced into cells, in vitro or in vivo. The present invention provides methods, reagents, vectors, and cells useful for expression of ovarian cancer-associated polypeptides and nucleic acids using in vitro (cell-free), ex vivo or in vivo (cell or organism-based) recombinant expression systems.

The particular procedure used to introduce the nucleic acids into a host cell for expression of a protein or nucleic acid is application specific. Many procedures for introducing foreign nucleotide sequences into host cells may be used. These include the use of calcium phosphate transfection, spheroplasts, electroporation, liposomes, microinjection, plasma vectors, viral vectors and any of the other well known methods for introducing cloned genomic DNA, cDNA, synthetic DNA or other foreign genetic material into a host cell (see, e.g., Berger & Kimmel, Guide to Molecular Cloning Techniques, Methods in Enzymology volume 152 (Berger), Ausubel et al., eds., Current Protocols (supplemented through 1999), and Sambrook et al., Molecular Cloning—A Laboratory Manual (2nd ed., Vol. 1-3, 1989.

In a preferred embodiment, ovarian cancer proteins and modulators are administered as therapeutic agents, and can be formulated as outlined above. Similarly, ovarian cancer genes (including both the full-length sequence, partial sequences, or regulatory sequences of the ovarian cancer coding regions) can be administered in a gene therapy application. These ovarian cancer genes can include antisense applications, either as gene therapy (i.e. for incorporation into the genome) or as antisense compositions, as will be appreciated by those in the art.

Ovarian cancer polypeptides and polynucleotides can also be administered as vaccine compositions to stimulate HTL, CTL and antibody responses. Such vaccine compositions can include, e.g., lipidated peptides (see, e.g., Vitiello, A. et al., J. Clin. Invest. 95:341 (1995)), peptide compositions encapsulated in poly(DL-lactide-co-glycolide) (“PLG”) microspheres (see, e.g., Eldridge, et al., Molec. Immunol. 28:287-294, (1991); Alonso et al., Vaccine 12:299-306 (1994); Jones et al., Vaccine 13:675-681 (1995)), peptide compositions contained in immune stimulating complexes (ISCOMS) (see, e.g., Takahashi et al., Nature 344:873-875 (1990); Hu et al., Clin Exp Immunol. 113:235-243 (1998)), multiple antigen peptide systems (MAPs) (see, e.g., Tam, Proc. Natl. Acad. Sci. U.S.A. 85:5409-5413 (1988); Tam, J. Immunol. Methods 196:17-32 (1996)), peptides formulated as multivalent peptides; peptides for use in ballistic delivery systems, typically crystallized peptides, viral delivery vectors (Perkus, et al., In: Concepts in vaccine development (Kaufmann, ed., p. 379, 1996); Chakrabarti, et al., Nature 320:535 (1986); Hu et al., Nature 320:537 (1986); Kieny, et al., AIDS Bio/Technology 4:790 (1986); Top et al., J. Infect. Dis. 124:148 (1971); Chanda et al., Virology 175:535 (1990)), particles of viral or synthetic origin (see, e.g., Kofler et al., J. Immunol. Methods. 192:25 (1996); Eldridge et al., Sem. Hematol. 30:16 (1993); Falo et al., Nature Med. 7:649 (1995)), adjuvants (Warren et al., Annu. Rev. Immunol. 4:369 (1986); Gupta et al., Vaccine 11:293 (1993)), liposomes (Reddy et al., J. Immunol. 148:1585 (1992); Rock, Immunol. Today 17:131 (1996)), or, naked or particle absorbed cDNA (Ulmer, et al., Science 259:1745 (1993); Robinson et al., Vaccine 11:957 (1993); Shiver et al., In: Concepts in vaccine development (Kaufmann, ed., p. 423, 1996); Cease & Berzofsky, Annu. Rev. Immunol. 12:923 (1994) and Eldridge et al., Sem. Hematol. 30:16 (1993)). Toxin-targeted delivery technologies, also known as receptor mediated targeting, such as those of Avant Immunotherapeutics, Inc. (Needham, Mass.) may also be used.

Vaccine compositions often include adjuvants. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, e.g., Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Vaccines can be administered as nucleic acid compositions wherein DNA or RNA encoding one or more of the polypeptides, or a fragment thereof, is administered to a patient. This approach is described, for instance, in Wolff et. al., Science 247:1465 (1990) as well as U.S. Pat. Nos. 5,580,859; 5,589,466; 5,804,566; 5,739,118; 5,736,524; 5,679,647; WO 98/04720; and in more detail below. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

For therapeutic or prophylactic immunization purposes, the peptides of the invention can be expressed by viral or bacterial vectors. Examples of expression vectors include attenuated viral hosts, such as vaccinia or fowlpox. This approach involves the use of vaccinia virus, e.g., as a vector to express nucleotide sequences that encode ovarian cancer polypeptides or polypeptide fragments. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization e.g. adeno and adeno-associated virus vectors, retroviral vectors, Salmonella typhi vectors, detoxified anthrax toxin vectors, and the like, will be apparent to those skilled in the art from the description herein (see, e.g., Shata et al., Mol Med Today 6:66-71 (2000); Shedlock et al., J Leukoc Biol 68:793-806 (2000); Hipp et al., In Vivo 14:571-85 (2000)).

Methods for the use of genes as DNA vaccines are well known, and include placing a ovarian cancer gene or portion of a ovarian cancer gene under the control of a regulatable promoter or a tissue-specific promoter for expression in a ovarian cancer patient. The ovarian cancer gene used for DNA vaccines can encode full-length ovarian cancer proteins, but more preferably encodes portions of the ovarian cancer proteins including peptides derived from the ovarian cancer protein. In one embodiment, a patient is immunized with a DNA vaccine comprising a plurality of nucleotide sequences derived from a ovarian cancer gene. For example, ovarian cancer-associated genes or sequence encoding subfragments of a ovarian cancer protein are introduced into expression vectors and tested for their immunogenicity in the context of Class I MHC and an ability to generate cytotoxic T cell responses. This procedure provides for production of cytotoxic T cell responses against cells which present antigen, including intracellular epitopes.

In a preferred embodiment, the DNA vaccines include a gene encoding an adjuvant molecule with the DNA vaccine. Such adjuvant molecules include cytokines that increase the immunogenic response to the ovarian cancer polypeptide encoded by the DNA vaccine. Additional or alternative adjuvants are available.

In another preferred embodiment ovarian cancer genes find use in generating animal models of ovarian cancer. When the ovarian cancer gene identified is repressed or diminished in cancer tissue, gene therapy technology, e.g., wherein antisense RNA directed to the ovarian cancer gene will also diminish or repress expression of the gene. Animal models of ovarian cancer find use in screening for modulators of a ovarian cancer-associated sequence or modulators of ovarian cancer. Similarly, transgenic animal technology including gene knockout technology, e.g. as a result of homologous recombination with an appropriate gene targeting vector, will result in the absence or increased expression of the ovarian cancer protein. When desired, tissue-specific expression or knockout of the ovarian cancer protein may be necessary.

It is also possible that the ovarian cancer protein is overexpressed in ovarian cancer. As such, transgenic animals can be generated that overexpress the ovarian cancer protein. Depending on the desired expression level, promoters of various strengths can be employed to express the transgene. Also, the number of copies of the integrated transgene can be determined and compared for a determination of the expression level of the transgene. Animals generated by such methods find use as animal models of ovarian cancer and are additionally useful in screening for modulators to treat ovarian cancer.

Kits for Use in Diagnostic and/or Prognostic Applications

For use in diagnostic, research, and therapeutic applications suggested above, kits are also provided by the invention. In the diagnostic and research applications such kits may include any or all of the following: assay reagents, buffers, ovarian cancer-specific nucleic acids or antibodies, hybridization probes and/or primers, antisense polynucleotides, ribozymes, dominant negative ovarian cancer polypeptides or polynucleotides, small molecules inhibitors of ovarian cancer-associated sequences etc. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for screening for modulators of ovarian cancer-associated sequences. Such kits can be prepared from readily available materials and reagents. For example, such kits can comprise one or more of the following materials: a ovarian cancer-associated polypeptide or polynucleotide, reaction tubes, and instructions for testing ovarian cancer-associated activity. Optionally, the kit contains biologically active ovarian cancer protein. A wide variety of kits and components can be prepared according to the present invention, depending upon the intended user of the kit and the particular needs of the user. Diagnosis would typically involve evaluation of a plurality of genes or products. The genes will be selected based on correlations with important parameters in disease which may be identified in historical or outcome data.

### EXAMPLES

### Example 1

### Tissue Preparation, Labeling Chips, and Fingerprints

Purifying Total RNA from Tissue Sample Using TRIzol Reagent

The sample weight is first estimated. The tissue samples are homogenized in 1 ml of TRIzol per 50 mg of tissue using a homogenizer (e.g., Polytron 3100). The size of the generator/probe used depends upon the sample amount. A generator that is too large for the amount of tissue to be homogenized will cause a loss of sample and lower RNA yield. A larger generator (e.g., 20 mm) is suitable for tissue samples weighing more than 0.6 g. Fill tubes should not be overfilled. If the working volume is greater than 2 ml and no greater than 10 ml, a 15 ml polypropylene tube (Falcon 2059) is suitable for homogenization.

Tissues should be kept frozen until homogenized. The TRIzol is added directly to the frozen tissue before homogenization. Following homogenization, the insoluble material is removed from the homogenate by centrifugation at 7500×g for 15 min. in a Sorvall superspeed or 12,000×g for 10 min. in an Eppendorf centrifuge at 4° C. The cleared homogenate is then transferred to a new tube(s). Samples may be frozen and stored at −60 to −70° C. for at least one month or else continue with the purification.

The next process is phase separation. The homogenized samples are incubated for 5 minutes at room temperature. Then, 0.2 ml of chloroform per 1 ml of TRIzol reagent is added to the homogenization mixture. The tubes are securely capped and shaken vigorously by hand (do not vortex) for 15 seconds. The samples are then incubated at room temp. for 2-3 minutes and next centrifuged at 6500 rpm in a Sorvall superspeed for 30 min. at 4° C.

The next process is RNA Precipitation. The aqueous phase is transferred to a fresh tube. The organic phase can be saved if isolation of DNA or protein is desired. Then 0.5 ml of isopropyl alcohol is added per 1 ml of TRIzol reagent used in the original homogenization. Then, the tubes are securely capped and inverted to mix. The samples are then incubated at room temp. for 10 minutes an centrifuged at 6500 rpm in Sorvall for 20 min. at 4° C.

The RNA is then washed. The supernatant is poured off and the pellet washed with cold 75% ethanol. 1 ml of 75% ethanol is used per 1 ml of the TRIzol reagent used in the initial homogenization. The tubes are capped securely and inverted several times to loosen pellet without vortexing. They are next centrifuged at <8000 rpm (<7500×g) for 5 minutes at 4° C.

The RNA wash is decanted. The pellet is carefully transferred to an Eppendorf tube (sliding down the tube into the new tube by use of a pipet tip to help guide it in if necessary). Tube(s) sizes for precipitating the RNA depending on the working volumes. Larger tubes may take too long to dry. Dry pellet. The RNA is then resuspended in an appropriate volume (e.g., 2-5 ug/ul) of DEPC H₂O. The absorbance is then measured.

The poly A+ mRNA may next be purified from total RNA by other methods such as Qiagen's RNeasy kit. The poly A+ mRNA is purified from total RNA by adding the oligotex suspension which has been heated to 37° C. and mixing prior to adding to RNA. The Elution Buffer is incubated at 70° C. If there is precipitate in the buffer, warm up the 2×Binding Buffer at 65° C. The the total RNA is mixed with DEPC-treated water, 2×Binding Buffer, and Oligotex according to Table 2 on page 16 of the Oligotex Handbook and next incubated for 3 minutes at 65° C. and 10 minutes at room temperature.

The preparation is centrifuged for 2 minutes at 14,000 to 18,000 g, preferably, at a “soft setting,” The supernatant is removed without disturbing Oligotex pellet. A little bit of solution can be left behind to reduce the loss of Oligotex. The supernatant is saved until satisfactory binding and elution of poly A+ mRNA has been found.

Then, the preparation is gently resuspended in Wash Buffer OW2 and pipetted onto the spin column and centrifuged at full speed (soft setting if possible) for 1 minute.

Next, the spin column is transferred to a new collection tube and gently resuspended in Wash Buffer OW2 and centrifuged as described herein.

Then, the spin column is transferred to a new tube and eluted with 20 to 100 ul of preheated (70° C.) Elution Buffer. The Oligotex resin is gently resuspended by pipetting up and down. The centrifugation is repeated as above and the elution repeated with fresh elution buffer or first eluate to keep the elution volume low.

The absorbance is next read to determine the yield, using diluted Elution Buffer as the blank.

Before proceeding with cDNA synthesis, the mRNA is precipitated before proceeding with cDNA synthesis, as components leftover or in the Elution Buffer from the Oligotex purification procedure will inhibit downstream enzymatic reactions of the mRNA. 0.4 vol. of 7.5 M NH4OAc+2.5 vol. of cold 100% ethanol is added and the preparation precipitated at −20° C. 1 hour to overnight (or 20-30 min. at −70° C.), and centrifuged at 14,000-16,000×g for 30 minutes at 4° C. Next, the pellet is washed with 0.5 ml of 80% ethanol (−20° C.) and then centrifuged at 14,000-16,000×g for 5 minutes at room temperature. The 80% ethanol wash is then repeated. The last bit of ethanol from the pellet is then dried without use of a speed vacuum and the pellet is then resuspended in DEPC H₂O at 1 ug/ul concentration.

Alternatively the RNA may be Purified Using Other Methods (e.g., Qiagen's RNeasy Kit).

No more than 100 ug is added to the RNeasy column. The sample volume is adjusted to 100 ul with RNase-free water. 350 ul Buffer RLT and then 250 ul ethanol (100%) are added to the sample. The preparation is then mixed by pipetting and applied to an RNeasy mini spin column for centrifugation (15 sec at >10,000 rpm). If yield is low, reapply the flowthrough to the column and centrifuge again.

Then, transfer column to a new 2 ml collection tube and add 500 ul Buffer RPE and centrifuge for 15 sec at >10,000 rpm. The flowthrough is discarded. 500 ul Buffer RPE and is then added and the preparation is centriuged for 15 sec at >10,000 rpm. The flowthrough is discarded. and the column membrane dried by centrifuging for 2 min at maximum speed. The column is transferred to a new 1.5-ml collection tube. 30-50 ul of RNase-free water is applied directly onto column membrane. The column is then centrifuged for 1 min at >10,000 rpm and the elution step repeated.

The absorbance is then read to determine yield. If necessary, the material may be ethanol precipitated with ammonium acetate and 2.5×volume 100% ethanol.

First Strand cDNA Synthesis

The first strand can be make using using Gibco's “SuperScript Choice System for cDNA Synthesis” kit. The starting material is 5 ug of total RNA or 1 ug of polyA+ mRNA1. For total RNA, 2 ul of SuperScript RT is used; for polyA+ mRNA, 1 ul of SuperScript RT is used. The final volume of first strand synthesis mix is 20 ul. The RNA should be in a volume no greater than 10 ul. The RNA is incubated with 1 ul of 100 pmol T7-T24 oligo for 10 min at 70° C. followed by addition on ice of 7 ul of: 4 ul 5×1^{st} Strand Buffer, 2 ul of 0.1M DTT, and 1 ul of 10 mM dNTP mix. The preparation is then incubated at 37° C. for 2 min before addition of the SuperScript RT followed by incubation at 37° C. for 1 hour.

Second Strand Synthesis

For the second strand synthesis, place 1st strand reactions on ice and add: 91 ul DEPC H₂O; 30 ul 5×2nd Strand Buffer; 3 ul 10 mM dNTP mix; 1 ul 10 U/ul E.coli DNA Ligase; 4 ul 10 U/ul E.coli DNA Polymerase; and 1 ul 2 U/ul RNase H. Mix and incubate 2 hours at 16° C. Add 2 ul T4 DNA Polymerase. Incubate 5 min at 16° C. Add 10 ul of 0.5M EDTA.

Cleaning up cDNA

The cDNA is purified using Phenol:Chloroform:Isoamyl Alcohol (25:24:1) and Phase-Lock gel tubes. The PLG tubes are centrifuged for 30 sec at maximum speed. The cDNA mix is then transferred to PLG tube. An equal volume of phenol:chloroform:isamyl alcohol is then added, the preparation shaken vigorously (no vortexing), and centrifuged for 5 minutes at maximum speed. The top aqueous solution is transferred to a new tube and ethanol precipitated by adding 7.5×5M NH4OAc and 2.5×volume of 100% ethanol. Next, it is centrifuged immediately at room temperature for 20 min, maximum speed. The supernatant is removed, and the pellet washed with 2×with cold 80% ethanol. As much ethanol wash as possible should be removed before air drying the pellet; and resuspending it in 3 ul RNase-free water.

In vitro Transcription (IVT) and Labeling with Biotin

In vitro Transcription (IVT) and labeling with biotin is performed as follows: Pipet 1.5 ul of cDNA into a thin-wall PCR tube. Make NTP labeling mix by combining 2 ul T7 10×ATP (75 mM) (Ambion); 2 ul T7 10×GTP (75 mM) (Ambion); 1.5 ul T7 10×CTP (75 mM) (Ambion); 1.5 ul T7 10×UTP (75 mM) (Ambion); 3.75 ul 10 mM Bio-1 1-UTP (Boehringer-Mannheim/Roche or Enzo); 3.75 ul 10 mM Bio-16-CTP (Enzo); 2 ul 10×T7 transcription buffer (Ambion); and 2 ul 10×T7 enzyme mix (Ambion). The final volume is 20 ul. Incubate 6 hours at 37° C. in a PCR machine. The RNA can be furthered cleaned. Clean-up follows the previous instructions for RNeasy columns or Qiagen's RNeasy protocol handbook. The cRNA often needs to be ethanol precipitated by resuspension in a volume compatible with the fragmentation step.

Fragmentation is performed as follows. 15 ug of labeled RNA is usually fragmented. Try to minimize the fragmentation reaction volume; a 10 ul volume is recommended but 20 ul is all right. Do not go higher than 20 ul because the magnesium in the fragmentation buffer contributes to precipitation in the hybridization buffer. Fragment RNA by incubation at 94 C for 35 minutes in 1×Fragmentation buffer (5×Fragmentation buffer is 200 mM Tris-acetate, pH 8.1; 500 mM KOAc; 150 mM MgOAc). The labeled RNA transcript can be analyzed before and after fragmentation. Samples can be heated to 65° C. for 15 minutes and electrophoresed on 1% agarose/TBE gels to get an approximate idea of the transcript size range For hybridization, 200 ul (10 ug cRNA) of a hybridization mix is put on the chip. If multiple hybridizations are to be done (such as cycling through a 5 chip set), then it is recommended that an initial hybridization mix of 300 ul or more be made. The hybridization mix is: fragment labeled RNA (50 ng/ul final conc.); 50 pM 948-b control oligo; 1.5 pM BioB; 5 pM BioC; 25 pM BioD; 100 pM CRE; 0.1 mg/ml herring sperm DNA; 0.5 mg/ml acetylated BSA; and 300 ul with 1×MES hyb buffer.

The hybridization reaction is conducted with non-biotinylated IVT (purified by RNeasy columns) (see example 1 for steps from tissue to IVT): The following mixture is prepared:

| | |
|---|---|
| IVT antisense RNA; 4 μg: | μl |
| Random Hexamers (1 μg/μl): | 4 μl |
| H₂O: | μl |

Incubate the above 14 μl mixture at 70° C. for 10 min.; then put on ice.

The Reverse transcription procedure uses the following mixture:

| | |
|---|---|
| 0.1 M DTT: | 3 μl |
| 50X dNTP mix: | 0.6 μl |
| H₂O: | 2.4 μl |
| Cy3 or Cy5 dUTP (1mM): | 3 μl |
| SS RT II (BRL): | 1 μl |
| | 16 μl |

The above solution is added to the hybridization reaction and incubated for 30 min., 42° C. Then, 1 μl SSII is added and incubated for another hour before being placed on ice.

The 50×dNTP mix contains 25 mM of cold dATP, dCTP, and dGTP, 10 mM of dTTP and is made by adding 25 μl each of 100 mM dATP, dCTP, and dGTP; 10 μl of 100 mM dTTP to 15 μl H₂O.]

RNA degradation is performed as follows. Add 86 μl H₂O, 1.5 μl 1M NaOH/2 mM EDTA and incubate at 65° C., 10 min. For U-Con 30, 500 μl TE/sample spin at 7000 g for 10 min, save flow through for purification. For Qiagen purification, suspend u-con recovered material in 500 μl buffer PB and proceed using Qiagen protocol. For DNAse digestion, add 1 ul of 1/100 dilution of DNAse/30 ul Rx and incubate at 37° C. for 15 min. Incubate at 5 min 95° C. to denature the DNAse.

Sample Preparation

For sample preparation, add Cot-1 DNA, 10 μl; 50×dNTPs, 1 μl; 20×SSC, 2.3 μl; Na pyro phosphate, 7.5 μl; 10 mg/ml Herring sperm DNA; 1 ul of 1/10 dilution to 21.8 final vol. Dry in speed vac. Resuspend in 15 μl H₂O. Add 0.38 μl 10% SDS. Heat 95° C., 2 min and slow cool at room temp. for 20 min. Put on slide and hybridize overnight at 64° C. Washing after the hybridization: 3×SSC/0.03% SDS: 2 min., 37.5 ml 20×SSC+0.75 ml 10% SDS in 250 ml H₂O; 1×SSC: 5 min., 12.5 mls 20×SSC in 250 ml H₂O; 0.2×SSC: 5 min., 2.5 ml 20×SSC in 250 ml H₂O. Dry slides and scan at appropiate PMT's and channels.

TABLE 1
695 UP-REGULATED GENES, OVARIAN CANCER VERSUS NORMAL ADULT TISSUES

| | | | | ratio: |
|---|---|---|---|---|
| | | | | tumor |
| Primekey | Exemplar | UniGene | | vs. |
| tissues | Accession | ID | Title | normal |
| 452838 | U65011 | Hs.30743 | Preferentially expressed antigen in melanoma | 70.4 |
| 438817 | AI023799 | Hs.163242 | ESTs | 62.8 |
| 432938 | T27013 | Hs.3132 | steroidogenic acute regulatory protein | 57.8 |
| 421478 | AI683243 | Hs.97258 | ESTs | 45.7 |
| 415989 | AI267700 | Hs.111128 | ESTs | 42.7 |
| 418179 | X51630 | Hs.1145 | Wilms tumor 1 | 36.0 |
| 449034 | AI624049 | | gb:ts41a09.x1 NCI_CGAP_Ut1 Homo sapiens cDNA clone | 34.0 |
| 428579 | NM_005756 | Hs.184942 | G protein-coupled receptor 64 | 30.5 |
| 428153 | AW513143 | Hs.98367 | hypothetical protein FLJ22252 similar to SRY-box c | 30.1 |
| 436982 | AB018305 | Hs.5378 | spondin 1 , (f-spondin) extracellular matrix protei | 29.4 |
| 427585 | D31152 | Hs.179729 | collagen; type X; alpha 1 (Schmid metaphyseal chon | 27.0 |
| 435094 | AI560129 | Hs.277523 | EST | 26.2 |
| 430691 | C14187 | Hs.103538 | ESTs | 26.2 |
| 430491 | AL109791 | Hs.241559 | Homo sapiens mRNA full length insert cDNA clone EU | 26.1 |
| 415511 | AI732617 | Hs.182362 | ESTs | 24.8 |
| 448243 | AW369771 | Hs.77496 | ESTs | 24.7 |
| 428187 | AI687303 | Hs.285529 | ESTs | 23.9 |
| 408081 | AW451597 | Hs.167409 | ESTs | 21.9 |
| 418007 | M13509 | Hs.83169 | Matrix metalloprotease 1 (interstitial collagenase | 20.6 |
| 400292 | AA250737 | Hs.72472 | BMPR-Ib; bone morphogenetic protein receptor; typ | 20.6 |
| 422956 | BE545072 | Hs.122579 | ESTs | 20.0 |
| 413335 | AI613318 | Hs.48442 | ESTs | 19.9 |
| 423739 | AA398155 | Hs.97600 | ESTs | 18.9 |
| 410929 | H47233 | Hs.30643 | ESTs | 18.5 |
| 424086 | AI351010 | Hs.102267 | lysyl oxidase | 17.7 |
| 424905 | NM_002497 | Hs.153704 | NIMA (never in mitosis gene a)-related kinase 2 | 17.4 |
| 427356 | AW023482 | Hs.97849 | ESTs | 17.4 |
| 407168 | R45175 | | gb:yg40f01.s1 Scares infant brain 1NIB Homo sapien | 17.1 |
| 407638 | AJ404672 | Hs.288693 | EST | 17.1 |
| 427469 | AA403084 | Hs.269347 | ESTs | 17.0 |
| 438993 | AA828995 | | integrin; beta 8 | 16.7 |
| 428664 | AK001666 | Hs.189095 | similar to SALL1 (sal (Drosophila)-like | 16.5 |
| 439820 | AL360204 | Hs.283853 | Homo sapiens mRNA full length insert cDNA clone EU | 16.5 |
| 421155 | H87879 | Hs.102267 | lysyl oxidase | 16.1 |
| 426635 | BE395109 | Hs.129327 | ESTs | 15.9 |
| 431989 | AW972870 | Hs.291069 | ESTs | 15.9 |
| 422805 | AA436989 | Hs.121017 | H2A histone family; member A | 15.9 |
| 444783 | AK001468 | Hs.62180 | ESTs | 15.8 |
| 424581 | M62062 | Hs.150917 | catenin (cadherin-associated protein), alpha 2 | 15.7 |
| 453197 | AI916269 | Hs.109057 | ESTs, Weakly similar to ALU5_HUMAN ALU SUBFAMIL | 15.7 |
| 459325 | AW088369 | Hs.282184 | ESTs | 15.6 |
| 428976 | AL037824 | Hs.194695 | ras homolog gene family, member I | 15.1 |
| 416209 | AA236776 | Hs.79078 | MAD2 (mitotic arrest deficient, yeast, homolog)-li | 15.0 |
| 408660 | AA525775 | Hs.292523 | ESTs | 15.0 |
| 410247 | AF181721 | Hs.61345 | RU2S | 15.0 |
| 418738 | AW388633 | Hs.6682 | solute carrier family 7, member 11 | 15.0 |
| 459583 | AI907673 | | gb:IL-BT152-080399-004 BT152 Homo sapiens cDNA, mR | 14.8 |
| 413623 | AA825721 | Hs.246973 | ESTs | 14.8 |
| 439706 | AW872527 | Hs.59761 | ESTs | 14.7 |
| 409041 | AB033025 | Hs.50081 | KIAA1199 protein | 14.6 |
| 451110 | AI955040 | Hs.301584 | ESTs | 14.5 |
| 436775 | AA731111 | Hs.291891 | ESTs | 14.3 |
| 443211 | AI128388 | Hs.143655 | ESTs | 14.3 |
| 445258 | AI635931 | Hs.147613 | ESTs | 14.2 |
| 447350 | AI375572 | Hs.172634 | ESTs; HER4 (c-erb-B4) | 14.2 |
| 428227 | AA321649 | Hs.2248 | INTERFERON-GAMMA INDUCED PROTEIN PRECURS | 14.1 |
| 453392 | U23752 | Hs.32964 | SRY (sex determining region Y)-box 11 | 13.9 |
| 447033 | AI357412 | Hs.157601 | EST - not in UniGene | 13.7 |
| 423811 | AW299598 | Hs.50895 | homeo box C4 | 13.7 |
| 452461 | N78223 | Hs.108106 | transcription factor | 13.7 |
| 451106 | BE382701 | Hs.25960 | N-myc | 13.6 |
| 416208 | AW291168 | Hs.41295 | ESTs | 13.5 |
| 452249 | BE394412 | Hs.61252 | ESTs | 13.4 |
| 452055 | AI377431 | Hs.293772 | ESTs | 13.2 |
| 439243 | AA593254 | Hs.191349 | ESTs | 13.1 |
| 420149 | AA255920 | Hs.88095 | ESTs | 12.9 |
| 429125 | AA446854 | Hs.271004 | ESTs | 12.9 |
| 413597 | AW302885 | Hs.117183 | ESTs | 12.8 |
| 416566 | NM_003914 | Hs.79378 | cyclin Al | 12.8 |
| 442438 | AA995998 | | gb:os26b03.sl NCI_CGAP_Kid5 Homo sapiens cDNA clon | 12.7 |
| 407710 | AW022727 | Hs.23616 | ESTs | 12.6 |
| 416661 | AA634543 | Hs.79440 | IGF-II mRNA-binding protein 3 | 12.6 |
| 428392 | HI0233 | Hs.2265 | secretory granule, neuroendocrine protein 1 (7B2 p | 12.4 |
| 431725 | X65724 | Hs.2839 | Norrie disease (pseudoghoma) | 12.3 |
| 447700 | AI420183 | Hs.171077 | ESTs, Weakly similar to similar to serine/threonin | 12.2 |
| 458027 | L49054 | Hs.85195 | ESTs, Highly similar to t(3;5)(q25.1;p34) fusion g | 12.2 |
| 408460 | AA054726 | Hs.285574 | ESTs | 12.2 |
| 424735 | U31875 | Hs.152677 | short-chain alcohol dehydrogenase family member | 12.0 |
| 415263 | AA948033 | Hs.130853 | ESTs | 11.9 |
| 400298 | AA032279 | Hs.61635 | STEAP1 | 11.8 |
| 452096 | BE394901 | Hs.226785 | ESTs | 11.7 |
| 421451 | AA291377 | Hs.50831 | ESTs | 11.6 |
| 435496 | AW840171 | Hs.265398 | ESTs, Weakly similar to transformation-related pro | 11.6 |
| 443715 | AI583187 | Hs.9700 | cyclin El | 11.5 |
| 402606 | #(NOCAT) | | | 11.5 |
| 436954 | AA740151 | Hs.130425 | ESTs | 11.5 |
| 413472 | BE242870 | Hs.75379 | solute carrier family 1 (glial high affinity gluta | 11.5 |
| 410102 | AW248508 | Hs.279727 | ESTs; | 11.4 |
| 408562 | AI436323 | Hs.31141 | Homo sapiens mRNA for KIAA1568 protein, partial cd | 11.4 |
| 452030 | AL137578 | Hs.27607 | Homo sapiens mRNA; cDNA DKFZp564N2464 (from clon | 11.4 |
| 442353 | BE379594 | Hs.49136 | ESTs | 11.3 |
| 427344 | NM_000869 | Hs.2142 | 5-hydroxytryptamine (serotonin) receptor 3A | 11.2 |
| 453160 | AI263307 | Hs.146228 | ESTs | 11.2 |
| 426427 | M86699 | Hs.169840 | TTK protein kinase | 11.1 |
| 449433 | AI672096 | Hs.9012 | ESTs | 11.1 |
| 412723 | AA648459 | Hs179912 | ESTs | 11.1 |
| 400250 | | | 0 | 11.1 |
| 419752 | AA249573 | Hs.152618 | ESTs | 11.1 |
| 438167 | R28363 | Hs.24286 | ESTs | 11.1 |
| 434539 | AW748078 | Hs.214410 | ESTs | 10.9 |
| 429918 | AW873986 | Hs.119383 | ESTs | 10.8 |
| 450375 | AA009647 | Hs.8850 | a disintegrin and metalloproteinase domain 12 (mel | 10.8 |
| 400289 | X07820 | Hs.2258 | Matrix Metalloproteinase 10 (Stromolysin 2) | 10.8 |
| 420900 | AL045633 | Hs.44269 | ESTs | 10.8 |
| 428758 | AA433988 | Hs.98502 | Homo sapiens cDNA FLJ14303 fis, clone PLACE2000132 | 10.8 |
| 446142 | AI754693 | Hs.145968 | ESTs | 10.7 |
| 421285 | NM_000102 | Hs.1363 | cytochrome P450, subfamily XVII (steroid 17-alpha- | 10.6 |
| 433496 | AF064254 | Hs.49765 | VERY-LONG-CHAIN ACYL-COA SYNTHETASE | 10.6 |
| 418506 | AA084248 | Hs.85339 | G protein-coupled receptor 39 | 10.5 |
| 433447 | U29195 | Hs.3281 | neuronal pentraxin II | 10.4 |
| 424188 | AW954552 | Hs.142634 | zinc finger protein | 10.4 |
| 414245 | BE148072 | Hs.75850 | WAS protein family, member 1 | 10.3 |
| 426462 | U59111 | Hs.169993 | dermatan sulphate proteoglycan 3 | 10.3 |
| 418601 | AA279490 | Hs.86368 | calmegin | 10.3 |
| 444170 | AW613879 | Hs.102408 | ESTs | 10.3 |
| 453616 | NM_003462 | Hs.33846 | dynein, axonemal, light intermediate polypeptide | 10.3 |
| 407378 | AA299264 | | gb:EST11752 Uterus Homo sapiens cDNA 5′ end simula | 10.2 |
| 440901 | AA909358 | Hs.128612 | ESTs | 10.2 |
| 407366 | AF026942 | | gb:Homo sapiens cig33 mRNA, partial sequence. | 10.2 |
| 415227 | AW821113 | Hs.72402 | ESTs | 10.2 |
| 409269 | AA576953 | Hs.22972 | Homo sapiens cDNAFLJ13352 fis, clone OVARC1002165 | 10.1 |
| 450480 | X82125 | Hs.25040 | zinc finger protein 239 | 10.1 |
| 419088 | AI538323 | Hs.77496 | ESTs | 10.0 |
| 453922 | AF053306 | Hs.36708 | budding uninhibited by benzimidazoles 1 (yeast horn | 9.9 |
| 428253 | AL133640 | Hs.183357 | Homo sapiens mRNA; cDNA DKFZp586C1021 (from clone | 9.8 |
| 426471 | M22440 | Hs.170009 | transforming growth factor, alpha | 9.8 |
| 407881 | AW072003 | Hs.40968 | heparan sulfate (glucosamine) 3-O-sulfotransferase | 9.7 |
| 452291 | AFO15592 | Hs.28853 | CDC7 (cell division cycle 7, S. cerevisiae, homolo | 9.7 |
| 445537 | AJ245671 | Hs.12844 | EGF-like-domain; multiple 6 | 9.7 |
| 442875 | BE623003 | Hs.23625 | Homo sapiens clone TCCCTA00142 mRNA sequence | 9.6 |
| 423992 | AW898292 | Hs.137206 | Homo sapiens mRNA; cDNA DKFZp564H1663 (from clon | 9.6 |
| 412140 | AA219691 | Hs.73625 | RAB6 interacting, kinesin-like (rabkinesm6) | 9.6 |
| 407721 | Y12735 | Hs.38018 | dual-specificity tyrosine-(Y)-phosphorylation regu | 9.6 |
| 438209 | AL120659 | Hs.6111 | KIAA0307 gene product | 9.5 |
| 429782 | NM_005754 | Hs.220689 | Ras-GTPase-activating protein SH3-domain-binding p | 9.5 |
| 424945 | AI221919 | Hs.173438 | hypothetical protein FLJ10582 | 9.5 |
| 414972 | BE263782 | Hs.77695 | KIAA0008 gene product | 9.4 |
| 439262 | AA832333 | Hs.124399 | ESTs | 9.4 |
| 403381 | #(NOCAT) | | 0 | 9.3 |
| 424834 | AK001432 | Hs.153408 | Homo sapiens cDNA FLJ10570 fis, clone NT2RP20031 17 | 9.3 |
| 435509 | AI458679 | Hs.181915 | ESTs | 9.3 |
| 445413 | AA151342 | Hs.12677 | CGI-147 protein | 9.2 |
| 414083 | AL121282 | Hs.257786 | ESTs | 9.2 |
| 421373 | AA808229 | Hs.167771 | ESTs | 9.2 |
| 430510 | AW162916 | Hs.241576 | hypothetical protein PRO2577 | 9.1 |
| 446999 | AA151520 | Hs.279525 | hypothetical protein PRO2605 | 9.1 |
| 459587 | AA031956 | | gb:zk15e04.s1 Soares_pregnant_urerus_NbHPU Homo sa | 9.1 |
| 414569 | AF109298 | Hs.118258 | Prostate cancer associated protein 1 | 9.1 |
| 406687 | M31126 | Hs.272620 | pregnancy specific beta-1-glycoprotein 9 | 9.0 |
| 428479 | Y00272 | Hs.184572 | cell division cycle 2, G1 to S and G2 to M | 9.0 |
| 408908 | BE296227 | Hs.48915 | serine/threonine kinase 15 | 9.0 |
| 431548 | AI834273 | Hs.9711 | Homo sapiens cDNA FLJ13018 fis, clone NT2RP3000685 | 9.0 |
| 433764 | AW753676 | Hs.39982 | ESTs | 9.0 |
| 434636 | AA083764 | Hs.241334 | ESTs | 8.9 |
| 451807 | W52854 | Hs.27099 | DKFZP564J0863 protein | 8.8 |
| 437872 | AK002015 | Hs.5887 | RNA binding motif protein 7 | 8.8 |
| 443054 | AI745185 | Hs.8939 | yes-associated protein 65 kDa | 8.8 |
| 420092 | AA814043 | Hs.88045 | ESTs | 8.8 |
| 420159 | AI572490 | Hs.99785 | ESTs | 8.8 |
| 447164 | AF026941 | Hs.17518 | Homo sapiens cig5 mRNA, partial sequence | 8.8 |
| 451254 | AI571016 | Hs.172967 | ESTs | 8.8 |
| 432677 | NM_004482 | Hs.278611 | UDP-N-acetyl-alpha-D-galactosamine:polypeptide N-a | 8.7 |
| 450434 | AA166950 | Hs.18645 | ESTs, Weakly similar to partial CDS [C. elegans] | 8.7 |
| 400301 | X03635 | Hs.1657 | Estrogen receptor 1 | 8.7 |
| 408829 | NM_006042 | Hs.48384 | heparan sulfate (glucosamine) 3-O-sulfotransferase | 8.7 |
| 434891 | AA814309 | Hs.123583 | ESTs | 8.7 |
| 436812 | AW298067 | | gb:UI-H-BW0-ajp-g-09-0-UI.s1 NCI_CGAP_Sub6 Homo s | 8.7 |
| 438885 | AI886558 | Hs.184987 | ESTs | 8.7 |
| 449765 | N92293 | Hs.206832 | EST, Moderately similar to ALU8_HUMAN ALU SUBFAM | 8.7 |
| 447342 | A1199268 | Hs.19322 | ESTs; Weakly similar to !!!! ALU SUBFAMILY J WARN1 | 8.6 |
| 434424 | AI811202 | Hs.125365 | Homo sapiens cDNA: FLJ23523 fis, clone LNG05548 | 8.6 |
| 438078 | AI016377 | Hs.131693 | ESTs | 8.6 |
| 437212 | AI765021 | Hs.210775 | ESTs | 8.5 |
| 417728 | AW138437 | Hs.24790 | KIAA1573 protein | 8.5 |
| 438081 | H49546 | Hs.298964 | ESTs | 8.5 |
| 411571 | AA122393 | Hs.70811 | hypothetical protein FLJ20516 | 8.4 |
| 435663 | AI023707 | Hs.134273 | ESTs | 8.4 |
| 424717 | H03754 | Hs.152213 | wingless-type MMTV integration site family, member | 8.4 |
| 425734 | AF056209 | Hs.159396 | peptidylglycine alpha-amidating monooxygenase COOH | 8.4 |
| 450505 | NM_004572 | Hs.25051 | plakophilin 2 | 8.4 |
| 436211 | AK001581 | Hs.80961 | polymerase (DNA directed), gamma | 8.3 |
| 436396 | AI683487 | Hs.299112 | Homo sapiens cDNA FLJ1 1441 fis, clone HEMBA1001323 | 8.3 |
| 425695 | NM_005401 | Hs.159238 | protein tyrosine phosphatase, non-receptor type 14 | 8.3 |
| 438180 | AA808189 | Hs.272151 | ESTs | 8.2 |
| 447268 | AI370413 | Hs.36563 | Homo sapiens cDNA: FLJ2241 8 fis, clone HRC08590 | 8.2 |
| 433159 | AB035898 | Hs.150587 | kinesin-like protein 2 | 8.1 |
| 400195 | | | 0 | 8.1 |
| 424906 | AI566086 | Hs.153716 | Homo sapiens mRNA for Hmob33 protein, 3′ untransla | 8.1 |
| 438202 | AW169287 | Hs.22588 | ESTs | 8.1 |
| 438915 | AA280174 | Hs.23282 | ESTs | 8.1 |
| 448776 | BE302464 | Hs.30057 | transporter similar to yeast MRS2 | 8.1 |
| 453884 | AA355925 | Hs.36232 | KIAA0186 gene product | 8.0 |
| 420757 | X78592 | Hs.99915 | androgen receptor (dihydrotestosterone receptor; t | 8.0 |
| 439759 | AL359055 | Hs.67709 | Homo sapiens mRNA full length insert cDNA clone EU | 8.0 |
| 453102 | NM_007197 | Hs.31664 | frizzled (Drosophila) homolog 10 | 8.0 |
| 424001 | W67883 | Hs.137476 | KIAA1051 protein | 8.0 |
| 434415 | BE177494 | | gb:RC6-HT0596-270300-011-C05 HT0596 Homo sapiens c | 8.0 |
| 417576 | AA339449 | Hs.82285 | phosphoribosylglycinamide formyltransferase, phosp | 7.9 |
| 438966 | AW979074 | | gb:EST391 184 MAGE resequences, MAGP Homo sapiens c | 7.9 |
| 415245 | N59650 | Hs.27252 | ESTs | 7.9 |
| 422352 | AA766296 | Hs.99200 | ESTs | 7.9 |
| 425492 | AL021918 | Hs.158174 | zinc finger protein 1 84 (Kruppel-like) | 7.8 |
| 442655 | AW027457 | Hs.30323 | ESTs | 7.8 |
| 445657 | AW612141 | Hs.279575 | ESTs | 7.8 |
| 450221 | AA328102 | Hs.24641 | cytoskeleton associated protein 2 | 7.8 |
| 426320 | W47595 | Hs.169300 | transforming growth factor, beta 2 | 7.8 |
| 414142 | AW368397 | Hs.150042 | ESTs | 7.7 |
| 412170 | D16532 | Hs.73729 | very low density lipoprotein receptor | 7.6 |
| 410011 | AB020641 | Hs.57856 | PFTAIRE protein kinase 1 | 7.6 |
| 436476 | AA326108 | Hs.53631 | ESTs | 7.6 |
| 414132 | AI801235 | Hs.48480 | ESTs | 7.6 |
| 437789 | AI581344 | Hs.127812 | ESTs, Weakly similar to AF141326 1 RNA helicase HD | 7.6 |
| 450192 | AA263143 | Hs24596 | RAD51-interacting protein | 7.6 |
| 449328 | AI962493 | Hs.197647 | ESTs | 7.5 |
| 440238 | AW451970 | Hs.155644 | paired box gene 2 | 7.5 |
| 403657 | #(NOCAT) | | 0 | 7.5 |
| 408826 | AF216077 | Hs.48376 | Homo sapiens clone HB-2 mRNA sequence | 7.5 |
| 418735 | N48769 | Hs.44609 | ESTs | 7.5 |
| 413627 | BE182082 | Hs.246973 | ESTs | 7.4 |
| 446293 | AI420213 | Hs.149722 | ESTs | 7.4 |
| 441627 | AA947552 | Hs.58086 | ESTs | 7.4 |
| 425465 | LI8964 | Hs.1904 | protein kinase C; iota | 7.3 |
| 409242 | AL080170 | Hs.51692 | DKFZP434C091 protein | 7.3 |
| 450262 | AW409872 | Hs.271166 | ESTs, Moderately similar to ALU7 HUMAN ALU SUBFA | 7.3 |
| 440250 | AA876179 | Hs.134650 | ESTs | 7.3 |
| 451659 | BE379761 | Hs.14248 | ESTs, Weakly similar to ALU8_HUMAN ALU SUBFAM IL | 7.3 |
| 458861 | AI630223 | | gb:ad06g08.r1 Proliferating Erythroid Cells (LCB:a | 7.3 |
| 436032 | AA150797 | Hs.109276 | latexin protein | 7.2 |
| 407771 | AL138272 | Hs.62713 | ESTs | 7.2 |
| 435039 | AW043921 | Hs.130526 | ESTs | 7.2 |
| 444342 | NM_014398 | Hs.10887 | similar to lysosome-associated membrane glycoprote | 7.2 |
| 407829 | AA045084 | Hs.29725 | Homo sapiens cDNA FLJ13197 fis, clone NT2RP3004451 | 7.2 |
| 40973 1 | AA125985 | Hs.56145 | thymosin, beta, identified in neuroblastoma cells | 7.2 |
| 404253 | #(NOCAT) | | 0 | 7.1 |
| 424120 | T80579 | Hs.290270 | ESTs | 7.1 |
| 429126 | AW172356 | Hs.99083 | ESTs | 7.1 |
| 413573 | AI733859 | Hs.149089 | ESTs | 7.1 |
| 421464 | AA291553 | Hs.190086 | ESTs | 7.0 |
| 430388 | AA356923 | Hs.240770 | nuclear cap binding protein subunit 2, 20kD | 7.0 |
| 437938 | AI950087 | | ESTs; Weakly similar to Gag-Pol polyprotein [M. mus | 7.0 |
| 420362 | U79734 | Hs.97206 | huntingtin interacting protein 1 | 7.0 |
| 444743 | AA045648 | Hs.11817 | nudix (nucleoside diphosphate linked moiety X)-typ | 7.0 |
| 415138 | C18356 | Hs.78045 | tissue factor pathway inhibitor 2 TFPI2 | 6.9 |
| 410568 | AW162948 | Hs.64542 | pre-mRNA cleavage factor Im (68 kD) | 6.9 |
| 429418 | AI381028 | Hs.99283 | ESTs | 6.9 |
| 409178 | BE393948 | Hs.50915 | kallikrein 5 | 6.9 |
| 446608 | N75217 | Hs.257846 | ESTs | 6.9 |
| 425905 | AB032959 | Hs.161700 | KIAA1133 protein | 6.9 |
| 428532 | API57326 | Hs.184786 | TBP-interacting protein | 6.9 |
| 433426 | H69125 | Hs.133525 | ESTs | 6.9 |
| 431322 | AW970622 | | gb:EST382704 MAGE resequences, MAGK Homo sapiens | 6.8 |
| 437960 | AI669586 | Hs.222194 | ESTs | 6.8 |
| 423244 | AL039379 | Hs.209602 | ESTs, Weakly similar to ubiquitous TPR motif, Y is | 6.8 |
| 424085 | NM_002914 | Hs.139226 | replication factor C (activator 1) 2 (40 kD) | 6.8 |
| 448674 | W31178 | Hs.154140 | ESTs | 6.8 |
| 438122 | AI620270 | Hs.129837 | ESTs | 6.8 |
| 440048 | AA897461 | Hs.158469 | ESTs, Weakly similar to envelope protein [H. sapien | 6.7 |
| 418478 | U38945 | Hs.1174 | cyclin-dependent kinase inhibitor 2A (melanoma, pi | 6.7 |
| 407162 | N63855 | Hs.142634 | zinc finger protein | 6.7 |
| 410804 | U64820 | Hs.66521 | Machado-Joseph disease (spinocerebellar ataxia 3, | 6.7 |
| 424639 | AI917494 | Hs.131329 | ESTs | 6.7 |
| 432415 | T16971 | Hs.289014 | ESTs | 6.7 |
| 421470 | R27496 | Hs.1378 | annexin A3 | 6.7 |
| 445459 | AI478629 | Hs.158465 | ESTs | 6.7 |
| 418203 | X54942 | Hs.83758 | CDC28 protein kinase 2 | 6.6 |
| 432809 | AA565509 | Hs.131703 | ESTs | 6.6 |
| 409234 | AI879419 | Hs.27206 | ESTs | 6.6 |
| 438394 | BE379623 | Hs.27693 | CGI- 124 protein | 6.6 |
| 452097 | AB002364 | Hs.27916 | ADAM-TS3; a dismtegrin-like and metalloproteas | 6.6 |
| 453745 | AA952989 | Hs.63908 | Homo sapiens HSPC3I6 mRNA, partial cds | 6.6 |
| 414136 | AA812434 | Hs.178227 | ESTs | 6.6 |
| 423248 | AA380177 | Hs.125845 | ribulose-5-phosphate-3-epimerase | 6.6 |
| 454018 | AW016892 | Hs.241652 | ESTs | 6.6 |
| 452281 | T93500 | Hs.28792 | ESTs | 6.5 |
| 424620 | AA101043 | Hs.151254 | kallikrein 7 (chymotryptic; stratum corneum) | 6.5 |
| 452594 | AU076405 | Hs.29981 | solute carrier family 26 (sulfate transporter), me | 6.5 |
| 434149 | Z43829 | Hs.19574 | ESTs, Weakly similar to katanin p80 subunit [H. sap | 6.5 |
| 425776 | U25128 | Hs.159499 | parathyroid hormone receptor 2 | 6.4 |
| 418677 | S83308 | Hs.87224 | SRY (sex determining region Y)-box 5 | 6.4 |
| 409517 | X90780 | Hs.54668 | troponin I, cardiac | 6.4 |
| 432666 | AW204069 | Hs.129250 | ESTs, Weakly similar to unnamed protein product [H | 6.4 |
| 448706 | AW291095 | Hs.21814 | class II cytokine receptor ZCYTOR7 | 6.4 |
| 429163 | AA884766 | | gb:am20a10.s1 Soares_NFL_T_GBC_S1 Homo sapiens cDN | 6.4 |
| 413582 | AW295647 | Hs.71331 | Homo sapiens cDNA: FLJ21971 fis, clone HEP05790 | 6.4 |
| 419917 | AA320068 | Hs.93701 | Homo sapiens mRNA; cDNA DKFZp434E232 (from clone | 6.4 |
| 424153 | AA451737 | Hs.141496 | MAGE-like 2 | 6.4 |
| 434265 | AA846811 | Hs.130554 | Homo sapiens cDNA: FLJ23089 fis, clone LNG07061 | 6.4 |
| 435082 | AA664273 | Hs.186104 | Homo sapiens cDNA FLJ13803 fis, clone THYRO1000187 | 6.4 |
| 441081 | AI584019 | Hs.169006 | ESTs, Moderately similar to plakophilin 2b [H. sapi | 6.4 |
| 443539 | AI076182 | Hs.134074 | ESTs | 6.4 |
| 443830 | A1142095 | Hs.143273 | ESTs | 6.4 |
| 452606 | N45202 | Hs.90012 | Homo sapiens cDNA: FLJ23441 fis, clone HSI00612 | 6.4 |
| 418384 | AW149266 | Hs.25130 | ESTs | 6.3 |
| 425371 | D49441 | Hs.155981 | mesothelin | 6.3 |
| 429441 | AJ224172 | Hs.204096 | lipophilin B (uteroglobin family member), prostate | 6.3 |
| 449048 | Z45051 | Hs.22920 | similar to S68401 (cattle) glucose induced gene | 6.3 |
| 437117 | AL049256 | Hs.122593 | ESTs | 6.3 |
| 449579 | AW207260 | Hs.134014 | prostate cancer associated protein 6 | 6.3 |
| 453370 | AI470523 | Hs.182356 | ESTs, Moderately similar to translation initiation | 6.3 |
| 426514 | BE616633 | Hs.301122 | bone morphogenetic protein 7 (osteogenic protein 1 | 6.3 |
| 415076 | NM_000857 | Hs.77890 | guanylate cyclase 1 , soluble, beta 3 | 6.3 |
| 408155 | AB014528 | Hs.43133 | KIAA0628 gene product | 6.2 |
| 452904 | AL157581 | Hs.30957 | Homo sapiens mRNA; cDNA DKFZp434E0626 (from clone | 6.2 |
| 439138 | AI742605 | Hs.193696 | ESTs | 6.2 |
| 457030 | AI301740 | Hs.173381 | dihydropyrimidinase-like 2 | 6.2 |
| 436281 | AW411194 | Hs.120051 | ESTs | 6.1 |
| 407385 | AA610150 | Hs.272072 | ESTs, Moderately similar to ALU7_HUMAN ALU SUBFA | 6.1 |
| 406815 | AA833930 | Hs.288036 | tRNA isopentenylpyrophosphate transferase | 6.1 |
| 430437 | AI768801 | Hs.169943 | Homo sapiens cDNA FLJ13569 fis, clone PLACE1008369 | 6.1 |
| 428743 | AL080060 | Hs.301549 | Homo sapiens mRNA; cDNA DKFZp564H172 (from clone | 6.1 |
| 415139 | AW975942 | Hs.48524 | ESTs | 6.1 |
| 417404 | NM_007350 | Hs.82101 | pleckstrin homology-like domain, family A, member | 6.1 |
| 433527 | AW235613 | Hs.133020 | ESTs | 6.1 |
| 449448 | D60730 | Hs.57471 | ESTs | 6.1 |
| 457733 | AW974812 | Hs.291971 | ESTs | 6.1 |
| 457979 | AA776655 | Hs.270942 | ESTs | 6.1 |
| 422867 | L32137 | Hs.1584 | cartilage oligomeric matrix protein | 6.0 |
| 423554 | M90516 | Hs.1674 | glutamine-fructose-6-phosphate transaminase 1 | 6.0 |
| 421502 | AF111856 | Hs.105039 | solute carrier family 34 (sodium phosphate), membe | 6.0 |
| 412733 | AA984472 | Hs.74554 | KIAA0080 protein | 6.0 |
| 422095 | AI868872 | Hs.288966 | ceruloplasmin (ferroxidase) | 6.0 |
| 449347 | AV649748 | Hs.295901 | ESTs | 6.0 |
| 440870 | AI687284 | Hs.150539 | Homo sapiens cDNA FLJ13793 fis, clone THYRO1000085 | 6.0 |
| 437478 | AL390172 | Hs.118811 | ESTs | 6.0 |
| 411598 | BE336654 | Hs.70937 | H3 histone family, member K | 6.0 |
| 418134 | AA397769 | Hs.86617 | ESTs | 6.0 |
| 418845 | AA852985 | Hs.89232 | chromobox homolog 5 (Drosophila HP1 alpha) | 6.0 |
| 452039 | AI922988 | Hs.172510 | ESTs | 6.0 |
| 410555 | U92649 | Hs.64311 | a dismtegrin and metalloproteinase domain 1 7 (turn | 5.9 |
| 412719 | AW016610 | Hs.129911 | ESTs | 5.9 |
| 410566 | AA373210 | Hs.43047 | Homo sapiens cDNA FLJ13585 fis, clone PLACE1009150 | 5.9 |
| 437099 | N77793 | Hs.48659 | ESTs, Highly similar to LMA1_HUMAN LAMININ ALPH | 5.9 |
| 453431 | AF094754 | Hs.32973 | glycine receptor, beta | 5.9 |
| 408920 | AL120071 | Hs.48998 | fibronectin leucine rich transmembrane protein 2 | 5.9 |
| 417866 | AW067903 | Hs.82772 | “collagen, type XI, alpha 1” | 5.9 |
| 420440 | NM_002407 | Hs.97644 | mammaglobin 2 | 5.9 |
| 430291 | AV660345 | Hs.238126 | CGI-49 protein | 5.9 |
| 405547 | #(NOCAT) | | 0 | 5.9 |
| 427510 | Z47542 | Hs.179312 | small nuclear RNA activating complex, polypeptide | 5.9 |
| 435793 | AB037734 | Hs.4993 | ESTs | 5.8 |
| 427975 | AI536065 | Hs.122460 | ESTs | 5.8 |
| 428949 | AA442153 | Hs.104744 | ESTs, Weakly similar to AF208855 1 BM-013 [H. sapie | 5.8 |
| 452693 | T79153 | Hs.48589 | zinc finger protein 228 | 5.8 |
| 440138 | AB033023 | Hs.6982 | hypothetical protein FLJ10201 | 5.8 |
| 421246 | AW582962 | Hs.300961 | ESTs, Highly similar to AF151805 1 CGI-47 protein | 5.8 |
| 445424 | AB028945 | Hs.12696 | cortactin SH3 domain-binding protein | 5.8 |
| 448186 | AA262105 | Hs.4094 | Homo sapiens cDNA FLJ14208 fis, clone NT2RP3003264 | 5.8 |
| 425154 | NM_001851 | Hs.154850 | collagen, type IX, alpha 1 | 5.7 |
| 419335 | AW960146 | Hs.284137 | Homo sapiens cDNA FLJ12888 fis, clone NT2RP2004081 | 5.7 |
| 420637 | AW976153 | | gb:EST388262 MAGE resequences, MAGN Homo sapiens | 5.7 |
| 431924 | AK000850 | Hs.272203 | Homo sapiens cDNA FLJ20843 fis, clone ADKA01954 | 5.7 |
| 446868 | AV660737 | Hs.135100 | ESTs | 5.7 |
| 452971 | AI873878 | Hs.91789 | ESTs | 5.7 |
| 428927 | AA441837 | Hs.90250 | ESTs | 5.7 |
| 425282 | AW163518 | Hs.155485 | huntingtin interacting protein 2 | 5.7 |
| 419247 | S65791 | Hs.89764 | fragile X mental retardation 1 | 5.7 |
| 445640 | AW969626 | Hs.31704 | ESTs, Weakly similar to KJAA0227 [H. sapiens] | 5.7 |
| 422938 | NM_001809 | Hs.1594 | centromere protein A (17kD) | 5.6 |
| 447078 | AW885727 | Hs.301570 | ESTs | 5.6 |
| 421247 | BE391727 | Hs.102910 | general transcription factor IIH, polypeptide 4 (5 | 5.6 |
| 407896 | D76435 | Hs.41154 | Zic family member 1 (odd-paired Drosophila homolog | 5.6 |
| 436556 | AI364997 | Hs.7572 | ESTs | 5.6 |
| 417830 | AW504786 | Hs.132808 | epithelial cell transforming sequence 2 oncogene | 5.6 |
| 429826 | N93266 | Hs.40747 | ESTs | 5.6 |
| 432030 | AI908400 | Hs.143789 | ESTs | 5.6 |
| 443270 | NM_004272 | Hs.9192 | Homer, neuronal immediate early gene, 1B | 5.5 |
| 453900 | AW003582 | Hs.226414 | ESTs, Weakly similar to ALU8_HUMAN ALU SUBFAMIL | 5.5 |
| 411096 | U80034 | Hs.68583 | mitochondrial intermediate peptidase | 5.5 |
| 419558 | AW953679 | Hs.278394 | ESTs | 5.5 |
| 427386 | AW836261 | Hs.177486 | amyloid beta (A4) precursor protein (protease nexi | 5.5 |
| 427961 | AW293165 | Hs.143134 | ESTs | 5.5 |
| 404561 | #(NOCAT) | | 0 | 5.5 |
| 429682 | NM_006306 | Hs.211602 | SMC1 (structural maintenance of chromosomes 1, yea | 5.5 |
| 407216 | N91773 | Hs.102267 | lysyl oxidase | 5.5 |
| 410658 | AW105231 | Hs.192035 | ESTs | 5.5 |
| 413930 | M86153 | Hs.75618 | RAB11 A, member RAS oncogene family | 5.5 |
| 414315 | Z24878 | | gb:HSB65D052 STRATAGENE Human skeletal muscle cD | 5.5 |
| 427878 | C05766 | Hs.181022 | CGI-07 protein | 5.5 |
| 431041 | AA490967 | Hs.105276 | ESTs | 5.5 |
| 441645 | AI222279 | Hs.201555 | ESTs | 5.5 |
| 428071 | AF212848 | Hs.182339 | transcription factor ESE-3B | 5.4 |
| 436406 | AW105723 | Hs.125346 | ESTs | 5.4 |
| 429181 | AW979104 | Hs.294009 | ESTs | 5.4 |
| 410909 | AW898161 | Hs.53112 | ESTs, Weakly similar to ALU8_HUMAN ALU SUBFAMIL | 5.4 |
| 424345 | AK001380 | Hs.145479 | Homo sapiens cDNA FLJ10518 fis, clone NT2RP20008 14 | 5.4 |
| 451996 | AW514021 | Hs.245510 | ESTs | 5.4 |
| 449318 | AW236021 | Hs.108788 | ESTs, Weakly similar to zeste [D. melanogaster] | 5.4 |
| 441433 | AA933809 | Hs.42746 | ESTs | 5.4 |
| 445495 | BE622641 | Hs.38489 | ESTs | 5.4 |
| 410153 | BE311926 | Hs.15830 | Homo sapiens cDNA FLJ12691 fis, clone NT2RM4002571 | 5.4 |
| 442611 | BE077155 | Hs.177537 | ESTs | 5.4 |
| 452401 | NM_007115 | Hs.29352 | tumor necrosis factor, alpha-induced protein 6 | 5.4 |
| 453161 | AA628608 | Hs.61656 | ESTs | 5.4 |
| 419948 | AB041035 | Hs.93847 | NADPH oxidase 4 | 5.3 |
| 427718 | AI798680 | Hs.25933 | ESTs | 5.3 |
| 453867 | AI929383 | Hs.108196 | HSPC037 protein | 5.3 |
| 422634 | NM_016010 | Hs.118821 | CGI-62 protein | 5.3 |
| 444478 | W07318 | Hs.240 | M-phase phosphoprotein 1 | 5.3 |
| 428002 | AA418703 | | gb:zv98c03.s1 Soares_NhHMPu S1 Homo sapiens cDNA c | 5.3 |
| 443486 | NM_003428 | Hs.9450 | zinc finger protein 84 (HPF2) | 5.3 |
| 451177 | AI969716 | Hs.13034 | ESTs | 5.3 |
| 408298 | AI745325 | Hs.271923 | ESTs; Moderately similar to !!!! ALU SUBFAMILY SB2 | 5.3 |
| 435867 | AA954229 | Hs.114052 | ESTs | 5.3 |
| 423698 | AA329796 | Hs.1098 | DKFZp434J1813 protein | 5.3 |
| 448543 | AW897741 | Hs.21380 | Homo sapiens mRNA; cDNA DKFZp586Pl 124 (from clone | 5.3 |
| 427660 | AI741320 | Hs.114121 | Homo sapiens cDNA: FLJ23228 fis, clone CAE06654 | 5.3 |
| 430345 | AK000282 | Hs.239681 | hypothetical protein FLJ20275 | 5.3 |
| 433222 | AW514472 | Hs.238415 | ESTs, Moderately similar to ALU8 HUMAN ALU SUBFA | 5.3 |
| 449532 | W74653 | Hs.271593 | ESTs | 5.3 |
| 452822 | X85689 | Hs.288617 | Homo sapiens cDNA: FLJ22621 fis, clone HSI05658 | 5.3 |
| 437641 | AA811452 | Hs.291911 | ESTs | 5.2 |
| 418379 | AA218940 | Hs.137516 | fidgetin-like 1 | 5.2 |
| 416530 | U62801 | Hs.79361 | kallikrein 6 (neurosin, zyme) | 5.2 |
| 433589 | AA886530 | Hs.188912 | ESTs | 5.2 |
| 409143 | AW025980 | Hs.138965 | ESTs | 5.2 |
| 410303 | AA324597 | Hs.21851 | Homo sapiens cDNA FLJ12900 fis, clone NT2RP2004321 | 5.2 |
| 413384 | NM_000401 | Hs.75334 | exostoses (multiple) 2 | 5.2 |
| 424698 | AA164366 | Hs.151973 | hypothetical protein FLJ10378 | 5.2 |
| 431229 | AA496479 | | gb:zv37h05.r1 Scares ovary tumor NbHOT Homo sapien | 5.2 |
| 433377 | AI752713 | Hs.43845 | ESTs | 5.2 |
| 445236 | AK001676 | Hs.12457 | hypothetical protein FLJ10814 | 5.2 |
| 406367 | #(NOCAT) | | 0 | 5.2 |
| 442500 | AI819068 | Hs.209122 | ESTs | 5.2 |
| 450101 | AV649989 | Hs.24385 | Human hbc647 mRNA sequence | 5.2 |
| 419140 | AI982647 | Hs.215725 | ESTs | 5.2 |
| 411078 | AI222020 | Hs.182364 | ESTs, Weakly similar to 25 kDa trypsin inhibitor [ | 5.2 |
| 423020 | AA383092 | Hs.1608 | replication protein A3 (14 kD) | 5.2 |
| 427061 | AB032971 | Hs.173392 | KIA All 45 protein | 5.2 |
| 439042 | AW979172 | | gb:EST391282 MAGE resequences, MAGP Homo sapiens c | 5.2 |
| 452930 | AW195285 | Hs.194097 | ESTs | 5.2 |
| 417791 | AW965339 | Hs.111471 | ESTs | 5.1 |
| 433277 | W27266 | Hs.151010 | ESTs | 5.1 |
| 447835 | AW591623 | Hs.164129 | ESTs | 5.1 |
| 434401 | AI864131 | Hs.71119 | Putative prostate cancer tumor suppressor | 5.1 |
| 437496 | AA452378 | Hs.170144 | Homo sapiens mRNA; cDNA DKFZp547J125 (from clone D | 5.1 |
| 418849 | AW474547 | Hs.53565 | ESTs, Weakly similar to B0491.1 [C. elegans] | 5.1 |
| 428093 | AW594506 | Hs.104830 | ESTs | 5.1 |
| 408621 | AI970672 | Hs.46638 | chromosome 11 open reading frame 8; fetal brain ( | 5.1 |
| 453096 | AW294631 | Hs.11325 | ESTs | 5.1 |
| 418852 | BE537037 | Hs.273294 | hypothetical protein FLJ20069 | 5.1 |
| 436787 | AA908554 | Hs.192756 | ESTs | 5.1 |
| 446577 | AB040933 | Hs.15420 | KIAA1500 protein | 5.1 |
| 437267 | AW511443 | Hs.258110 | ESTs | 5.0 |
| 419423 | D26488 | Hs.90315 | KIAA0007 protein | 5.0 |
| 404939 | | | 0 | 5.0 |
| 439052 | AF085917 | Hs.37921 | ESTs | 5.0 |
| 447020 | T27308 | Hs.16986 | hypothetical protein FLJ11046 | 5.0 |
| 453878 | AW964440 | Hs.19025 | ESTs | 5.0 |
| 410824 | AW994813 | Hs.33264 | ESTs | 5.0 |
| 427701 | AA411101 | Hs.221750 | ESTs | 5.0 |
| 424602 | AK002055 | Hs.301129 | Homo sapiens clone 23859 mRNA sequence | 5.0 |
| 430044 | AA464510 | Hs.152812 | EST cluster (not in UniGene) | 5.0 |
| 417423 | AA197341 | Hs.111164 | ESTs | 5.0 |
| 421477 | AI904743 | Hs.104650 | hypothetical protein FLJ10292 | 5.0 |
| 433384 | AI021992 | Hs.124244 | ESTs | 5.0 |
| 434160 | BE551196 | Hs.114275 | ESTs | 5.0 |
| 443555 | N71710 | Hs.21398 | ESTs, Moderately similar to GNPI HUMAN GLUCOSAM | 5.0 |
| 416198 | H27332 | Hs.99598 | ESTs | 4.9 |
| 424539 | L02911 | Hs.150402 | activin A receptor, type I | 4.9 |
| 436645 | AW023424 | Hs.156520 | ESTs | 4.9 |
| 417251 | AWO15242 | Hs.99488 | ESTs; Weakly similar to ORF YKR074w [S. cerevisiae] | 4.9 |
| 447207 | AA442233 | Hs.17731 | hypothetical protein FLJ12892 | 4.9 |
| 416565 | AW000960 | Hs.44970 | ESTs | 4.9 |
| 425292 | NM_005824 | Hs.155545 | 37 kDa leucine-rich repeat (LRR) protein | 4.9 |
| 435420 | AI928513 | Hs.59203 | ESTs | 4.9 |
| 435532 | AW291488 | Hs.117305 | ESTs | 4.9 |
| 443268 | AI800271 | Hs.129445 | hypothetical protein FLJ12496 | 4.9 |
| 446140 | AA356170 | Hs.26750 | Homo sapiens cDNA: FLJ21908 fis, clone HEP03830 | 4.9 |
| 452891 | N75582 | Hs.212875 | ESTs, Weakly similar to KIAA0357 [H. sapiens] | 4.9 |
| 431130 | NM_006103 | Hs.2719 | epididymis-specific; whey-acidic protein type; fou | 4.9 |
| 408938 | AA059013 | Hs.22607 | ESTs | 4.9 |
| 432842 | AW674093 | Hs.279525 | hypothetical protein PRO2605 | 4.9 |
| 436754 | AI061288 | Hs.133437 | ESTs, Moderately similar to gonadotropin inducible | 4.9 |
| 442573 | H93366 | Hs.7567 | Branched chain aminotransferase 1, cytosolic, U215 | 4.9 |
| 409049 | AI423132 | Hs.146343 | ESTs | 4.9 |
| 422475 | AL359938 | Hs.117313 | Meis (mouse) homolog 3 | 4.9 |
| 447112 | HI7800 | Hs.7154 | ESTs | 4.9 |
| 458627 | AW088642 | Hs.97984 | ESTs; Weakly similar to WASP-family protein [H. sap | 4.8 |
| 431689 | AA305688 | Hs.267695 | UDP-Gal:betaGlcNAc beta 1,3-galactosyltransferase, | 4.8 |
| 410530 | M25809 | Hs.64173 | ESTs, Highly similar to VAB1 HUMAN VACUOLAR AT | 4.8 |
| 429414 | AI783656 | Hs.202095 | empty spiracles (Drosophila) homolog 2 | 4.8 |
| 418882 | NM_004996 | Hs.89433 | ATP-binding cassette, sub-family C (CFTR/MRP), mem | 4.8 |
| 422505 | AL120862 | Hs.124165 | ESTs; (HSA)PAP protein (programmed cell death 9; | 4.8 |
| 425977 | R15138 | Hs.165570 | Homo sapiens clone 25052 mRNA sequence | 4.8 |
| 428555 | NM_002214 | Hs.184908 | integrin, beta 8 | 4.8 |
| 452909 | NM_015368 | Hs.30985 | pannexin 1 | 4.8 |
| 449535 | W15267 | Hs.23672 | low density lipoprotein receptor-related protein 6 | 4.8 |
| 452232 | AW020603 | Hs.271698 | ESTs | 4.8 |
| 409732 | NM_016122 | Hs.56148 | NY-REN-58 antigen | 4.8 |
| 415115 | AA214228 | Hs.127751 | hypothetical protein | 4.7 |
| 423161 | AL049227 | Hs.124776 | Homo sapiens mRNA; cDNA DKFZp564N1116 (from clon | 4.7 |
| 441085 | AW136551 | Hs.181245 | Homo sapiens cDNA FLJ12532 fis, clone NT2RM4000200 | 4.7 |
| 423575 | C18863 | Hs.163443 | ESTs | 4.7 |
| 415211 | R64730.comp | Hs.155986 | ESTs; Highly similar to SPERM SURFACE PROTEIN SP1 | 4.7 |
| 418804 | AA809632 | | gb:nz17h04.s1 NCI_CGAP_GCB1 Homo sapiens cDNA clo | 4.7 |
| 428405 | Y00762 | Hs.2266 | cholinergic receptor, nicotinic, alpha polypeptide | 4.7 |
| 432865 | AI753709 | Hs.152484 | ESTs | 4.7 |
| 433330 | AW207084 | Hs.132816 | ESTs | 4.7 |
| 453047 | AW023798 | Hs.286025 | ESTs | 4.7 |
| 421308 | AA687322 | Hs.192843 | ESTs | 4.7 |
| 456273 | AF154846 | Hs.1148 | zinc finger protein | 4.7 |
| 443933 | AI091631 | Hs.135501 | Homo sapiens two pore potassium channel KT3.3 | 4.7 |
| 434551 | BE387162 | Hs.280858 | ESTs, Highly similar to XPB_HUMAN DNA-REPAIR PRO | 4.7 |
| 440351 | AF030933 | Hs.7179 | RAD1 (S. pombe) homolog | 4.7 |
| 426300 | U15979 | Hs.169228 | delta-like homolog (Drosophila) | 4.7 |
| 453775 | NM_002916 | Hs.35120 | replication factor C (activator 1) 4 (37kD) | 4.7 |
| 446102 | AW168067 | Hs.252956 | ESTs | 4.7 |
| 420547 | AF155140 | Hs.98738 | gonadotropin-regulated testicular RNA helicase | 4.7 |
| 429486 | AF155827 | Hs.203963 | hypothetical protein FLJ10339 | 4.7 |
| 429944 | RI3949 | Hs.226440 | Homo sapiens clone 24881 mRNA sequence | 4.7 |
| 433042 | AW193534 | Hs.281895 | Homo sapiens cDNA FLJ11660 fis, clone HEMBA1004610 | 4.7 |
| 434988 | AI418055 | Hs.161160 | ESTs | 4.6 |
| 452571 | W31518 | Hs.34665 | ESTs | 4.6 |
| 434361 | AF129755 | Hs.117772 | ESTs | 4.6 |
| 406400 | #(NOCAT) | | 0 | 4.6 |
| 410227 | AB009284 | Hs.61152 | exostoses (multiple)-like 2 | 4.6 |
| 419945 | AW290975 | Hs.118923 | ESTs | 4.6 |
| 428301 | AW628666 | Hs.98440 | ESTs | 4.6 |
| 430153 | AW968128 | | gb:EST380338 MAGE resequences, MAGJ Homo sapiens c | 4.6 |
| 431349 | AA503653 | Hs.156942 | ESTs, Moderately similar to ALU2_HUMAN ALU SUBFA | 4.6 |
| 446254 | BE179829 | Hs.179852 | Homo sapiens cDNA FLJ12832 fis, clone NT2RP2003137 | 4.6 |
| 447505 | AL049266 | Hs.18724 | Homo sapiens mRNA; cDNA DKFZp564F093 (from clone | 4.6 |
| 448027 | AI458437 | Hs.177224 | ESTs | 4.6 |
| 449611 | AI970394 | Hs.197075 | ESTs | 4.6 |
| 459574 | AI741122 | Hs.101810 | Homo sapiens cDNA FLJ14232 fis, clone NT2RP4000035 | 4.6 |
| 409928 | AL137163 | Hs.57549 | hypothetical protein dJ473B4 | 4.6 |
| 409387 | AW384900 | Hs.123526 | ESTs | 4.6 |
| 424078 | AB006625 | Hs.139033 | paternally expressed gene 3 | 4.6 |
| 435244 | N77221 | Hs.187824 | ESTs | 4.6 |
| 404996 | #(NOCAT) | | 0 | 4.6 |
| 407905 | AW103655 | Hs.252905 | ESTs | 4.6 |
| 411560 | AW851186 | | gb:IL3-CT0220-150200-071-H05 CT0220 Homo sapiens c | 4.6 |
| 424341 | AA385074 | | gb:EST98673 Thyroid Homo sapiens cDNA 5′ end simil | 4.6 |
| 441675 | AI914329 | Hs.5461 | ESTs | 4.6 |
| 452172 | H00797 | Hs.133207 | Homo sapiens mRNA for KIAA1230 protein, partial cd | 4.6 |
| 420276 | AA290938 | Hs.190561 | ESTs, Highly similar to mosaic protein LR11 [H. sap | 4.5 |
| 402820 | #(NOCAT) | | 0 | 4.5 |
| 419699 | AA248998 | Hs.31246 | ESTs | 4.5 |
| 422529 | AW015128 | Hs.256703 | ESTs | 4.5 |
| 438018 | AK001160 | Hs.5999 | hypothetical protein FLJ10298 | 4.5 |
| 441826 | AW503603 | Hs.129915 | phosphotriesterase related | 4.5 |
| 453931 | AL121278 | Hs.25144 | ESTs | 4.5 |
| 435538 | AB011540 | Hs.4930 | low density lipoprotein receptor-related protein 4 | 4.5 |
| 457465 | AW301344 | Hs.195969 | ESTs | 4.5 |
| 418848 | AI820961 | Hs.193465 | ESTs | 4.5 |
| 408321 | AW405882 | Hs.44205 | cortistatin | 4.5 |
| 447499 | AW262580 | Hs.147674 | KIAA1621 protein | 4.5 |
| 424513 | BE385864 | Hs.149894 | mitochondrial translational initiation factor 2 | 4.5 |
| 432731 | R31178 | Hs.287820 | fibronectin 1 | 4.5 |
| 448275 | BE514434 | Hs.20830 | synaptic Ras GTPase activating protein 1 (homolog | 4.5 |
| 430371 | D87466 | Hs.240112 | KIAA0276 protein | 4.5 |
| 448693 | AW004854 | Hs.228320 | Homo sapiens cDNA: FLJ23537 fis, clone LNG07690 | 4.5 |
| 407289 | AA135159 | Hs.203349 | Homo sapiens cDNA FLJ12149 fis, clone MAMMA 100042 | 4.4 |
| 448141 | AI471598 | Hs.197531 | ESTs | 4.4 |
| 434699 | AA643687 | Hs.149425 | Homo sapiens cDNA FLJ1 1980 fis, clone HEMBB1001304 | 4.4 |
| 417718 | T86540 | Hs.193981 | ESTs | 4.4 |
| 436464 | AI016176 | Hs.269783 | ESTs, Weakly similar to ALU1_HUMAN ALU SUBFAMIL | 4.4 |
| 427528 | AU077143 | Hs.179565 | minichromosome maintenance deficient (S. cerevisia | 4.4 |
| 409092 | AI735283 | Hs.172608 | ESTs | 4.4 |
| 416241 | N52639 | Hs.32683 | ESTs | 4.4 |
| 432005 | AA524190 | Hs.120777 | ESTs, Weakly similar to ELL2_HUMAN RNA POLYMER | 4.4 |
| 440234 | AW117264 | Hs.126252 | ESTs | 4.4 |
| 448743 | AB032962 | Hs.21896 | KIAA1136 protein | 4.4 |
| 451389 | N73222 | Hs.21738 | KIAA1008 protein | 4.4 |
| 453331 | AI240665 | Hs.8895 | ESTs | 4.4 |
| 454036 | AA374756 | Hs.93560 | ESTs, Weakly similar to unnamed protein product [H | 4.4 |
| 448133 | AA723157 | Hs.73769 | folate receptor 1 (adult) | 4.4 |
| 429597 | NM_003816 | Hs.2442 | a disintegrin and metalloproteinase domain 9 (melt | 4.4 |
| 153279 | AW893940 | Hs.59698 | ESTs | 4.4 |
| 409459 | D86407 | Hs.54481 | low density lipoprotein receptor-related protein 8 | 4.4 |
| 431708 | AI698136 | Hs.108873 | ESTs | 4.4 |
| 433906 | AI167816 | Hs.43355 | ESTs | 4.4 |
| 437958 | BE139550 | Hs.121668 | ESTs | 4.4 |
| 141423 | AI793299 | Hs.126877 | ESTs | 4.4 |
| 429876 | AB028977 | Hs.225974 | KIAA1054 protein | 4.3 |
| 446770 | AV660309 | Hs.154986 | ESTs, Weakly similar to AF137386 1 plasmolipin [H. | 4.3 |
| 112078 | X69699 | Hs.73149 | paired box gene 8 | 4.3 |
| 422093 | AF151852 | Hs.111449 | CGI-94 protein | 4.3 |
| (23123 | NM_012247 | Hs.124027 | SELENOPHOSPHATE SYNTHETASE; Human selenium | 4.3 |
| 448390 | AL035414 | Hs.21068 | hypothetical protein | 4.3 |
| 453628 | AW243307 | Hs.170187 | ESTs | 4.3 |
| 449722 | BE280074 | Hs.23960 | cyclin B1 | 4.3 |
| 436679 | AI127483 | Hs.120451 | ESTs, Weakly similar to unnamed protein product [H | 4.3 |
| 431592 | R69016 | Hs.293871 | ESTs, Weakly similar to ALU1 HUMAN ALU SUBFAMIL | 4.3 |
| 432383 | AK000144 | Hs.274449 | Homo sapiens cDNA FLJ20137 fis, clone COL07137 | 4.3 |
| 419926 | AW900992 | Hs.93796 | DKFZP586D2223 protein | 4.3 |
| 452367 | U71207 | Hs.29279 | eyes absent (Drosophila) homolog 2 | 4.3 |
| 401644 | #(NOCAT) | | 0 | 4.3 |
| 410044 | BE566742 | Hs58169 | highly expressed in cancer, rich in leucine heptad | 4.3 |
| 413775 | AW409934 | Hs.75528 | nucleolar GTPase | 4.3 |
| 424296 | AI631874 | Hs.169391 | ESTs | 4.3 |
| 431118 | BE264901 | Hs.250502 | carbonic anhydrase VIII | 4.3 |
| 432201 | AI538613 | Hs.135657 | TMPRSS3a mRNA for senne protease (ECHOS1) (TADG-1 | 4.3 |
| 451073 | AI758905 | Hs.206063 | ESTs | 4.3 |
| 451592 | AI805416 | Hs.213897 | ESTs | 4.3 |
| 452453 | AI902519 | | gb:QV-BT009-101198-051 BT009 Homo sapiens cDNA, m | 4.3 |
| 441020 | W79283 | Hs.35962 | ESTs | 4.2 |
| 439024 | R96696 | Hs.35598 | ESTs | 4.2 |
| 453619 | H87648 | Hs.33922 | H. sapiens novel gene from PAC 11 7P20, chromosome 1 | 4.2 |
| 453459 | BE047032 | Hs.257789 | ESTs | 4.2 |
| 408427 | AW194270 | Hs.177236 | ESTs | 4.2 |
| 419311 | AA689591 | | gb.nv66a12.s1 NCI_CGAP_GCB1 Homo sapiens cDNA clo | 4.2 |
| 426460 | D79721 | Hs.183702 | Homo sapiens cDNA FLJ11752 fis, clone HEMBA1005582 | 4.2 |
| 444540 | AI693927 | Hs.265165 | ESTs | 4.2 |
| 452943 | BE247449 | Hs.31082 | hypothetical protein FLJ10525 | 4.2 |
| 453913 | AW004683 | Hs.233502 | ESTs | 4.2 |
| 417847 | AI521558 | Hs.288312 | Homo sapiens cDNA: FLJ22316 fis, clone HRC05262 | 4.1 |
| 428856 | AA436735 | Hs.183171 | Homo sapiens cDNA: FLJ22002 fis, clone HEP06638 | 4.1 |
| 428679 | AA431765 | | gb:zw80c03.s1 Soares_testis_NHT Homo sapiens cDNA | 4.1 |
| 441006 | AW605267 | Hs.7627 | CGI-60 protein | 4.1 |
| 436209 | AW850417 | Hs.254020 | ESTs, Moderately similar to unnamed protein produc | 4.1 |
| 446936 | HI0207 | Hs.47314 | ESTs | 4.1 |
| 406076 | AL390179 | Hs.137011 | Homo sapiens mRNA; cDNA DKFZp547P134 (from clone | 4.1 |
| 428819 | AL135623 | Hs.193914 | KIAA0575 gene product | 4.1 |
| 406671 | AA129547 | Hs.285754 | met proto-oncogene (hepatocyte growth factor recep | 4.1 |
| 418432 | M14156 | Hs.85112 | insulin-like growth factor 1 (somatomedia C) | 4.1 |
| 417048 | AI088775 | Hs.55498 | geranylgeranyl diphosphate synthase 1 | 4.1 |
| 431750 | AA514986 | Hs.283705 | ESTs | 4.1 |
| 439314 | AA382413 | Hs.178144 | ESTs | 4.1 |
| 448582 | AI538880 | Hs.94812 | ESTs | 4.1 |
| 449554 | AA682382 | Hs.59982 | ESTs | 4.1 |
| 455700 | BE068115 | | gb:CM1-BT0368-061299-060-g07 BT0368 Homo sapiens c | 4.1 |
| 409073 | AA063458 | | gb:zf71a07.sl Soares_pineal_gland N3HPG Homo sapie | 4.1 |
| 433929 | AI375499 | Hs.27379 | ESTs | 4.1 |
| 415457 | AW081710 | Hs.7369 | ESTs, Weakly similar to ALU1 HUMAN ALU SUBFAMIL | 4.1 |
| 444381 | BE387335 | Hs.283713 | ESTs | 4.1 |
| 451024 | AA442176 | | gb:zw63b08.rl Soares_total_fetus_Nb2HF8_9w Homo sa | 4.1 |
| 415539 | AI733881 | Hs.72472 | BMPR-Ib; bone morphogenetic protein receptor; typ | 4.1 |
| 421515 | Y11339 | Hs.105352 | GalNAc aIpha-2, 6-sialyltransferase I, long form | 4.1 |
| 420736 | AI263022 | Hs.82204 | ESTs | 4.1 |
| 453293 | AA382267 | Hs.10653 | ESTs | 4.1 |
| 409564 | AA045857 | Hs.54943 | fracture callus 1 (rat) homolog | 4.1 |
| 418378 | AW962081 | | gb:EST374154 MAGE resequences, MAGG Homo sapiens | 4.1 |
| 429628 | H09604 | Hs.13268 | ESTs | 4.1 |
| 439635 | AA477288 | Hs.94891 | Homo sapiens cDNA: FLJ22729 fis, clone HSI15685 | 4.1 |
| 440452 | AI925136 | Hs.55150 | ESTs, Weakly similar to CAYP_HUMAN CALCYPHOSIN | 4.1 |
| 443695 | AW204099 | Hs.112759 | ESTs, Weakly similar to AF 126780 1 retinal short-c | 4.1 |
| 448816 | AB033052 | Hs.22151 | KIAA1 226 protein | 4.1 |
| 452795 | AW392555 | Hs.18878 | hypothetical protein FLJ21620 | 4.1 |
| 443171 | BE281128 | Hs.9030 | TONDU | 4.1 |
| 425322 | U63630 | Hs.155637 | protein kinase; DNA-activated; catalytic polypepti | 4.1 |
| 442717 | R88362 | Hs.180591 | ESTs, Weakly similar to R06F6.5b [C. elegans] | 4.1 |
| 414747 | U30872 | Hs.77204 | centromere protein F (350/400kD, mitosin) | 4.1 |
| 417300 | AI765227 | Hs.55610 | solute carrier family 30 (zinc transporter), membe | 4.1 |
| 417389 | BE260964 | Hs.82045 | Midkine (neurite growth-promoting factor 2) | 4.1 |
| 448105 | AW591433 | Hs.170675 | ESTs, Weakly similar to TMS2_HUMAN TRANSMEMBR | 4.1 |
| 419131 | AA406293 | Hs.301622 | ESTs | 4.1 |
| 406348 | #(NOCAT) | | 0 | 4.1 |
| 419750 | AL079741 | Hs.183114 | Homo sapiens cDNA FLJ14236 fis, clone NT2RP4000515 | 4.1 |
| 419790 | U79250 | Hs.93201 | glycerol-3-phosphate dehydrogenase 2 (mitochondria | 4.1 |
| 420908 | AL049974 | Hs.100261 | Homo sapiens mRNA; cDNA DKFZp564B222 (from clone | 4.1 |
| 421039 | NM_003478 | Hs.101299 | cullin 5 | 4.1 |
| 426890 | AA393167 | Hs.41294 | ESTs | 4.1 |
| 428571 | NM_006531 | Hs.2291 | Probe hTg737 (polycystic kidney disease, autosomal | 4.1 |
| 452834 | AI638627 | Hs.105685 | ESTs | 4.1 |
| 428771 | AB028992 | Hs.193143 | KIAA 1069 protein | 4.0 |
| 437949 | U78519 | Hs.41654 | ESTs | 4.0 |
| 450568 | AL050078 | Hs.25159 | Homo sapiens cDNA FLJ10784 fis, clone NT2RP4000448 | 4.0 |
| 424081 | NM_006413 | Hs.139120 | ribonuclease P (30kD) | 4.0 |
| 418375 | NM_003081 | Hs.84389 | synaptosomal-associated protein, 25kD | 4.0 |
| 447204 | AI366881 | Hs.157897 | ESTs, Moderately similar to ALUC_HUMAN !!!! ALU CL | 4.0 |
| 407910 | AA650274 | Hs41296 | fibronectin leucine rich transmembrane protein 3 | 4.0 |
| 412314 | AA825247 | Hs.250899 | heat shock factor binding protein 1 | 4.0 |
| 436291 | BE568452 | Hs.5101 | ESTs; Highly similar to protein regulating cytokin | 4.0 |
| 450654 | AJ245587 | Hs.25275 | Kruppel-type zinc finger protein | 4.0 |
| 426991 | AK001536 | Hs.285803 | Homo sapiens cDNA FLJ12852 fis, clone NT2RP2003445 | 4.0 |
| 409365 | AA702376 | Hs.226440 | Homo sapiens clone 24881 mRNA sequence | 4.0 |
| 410784 | AW803201 | | gb:IL2-UM0077-070500-080-E06 UM0077 Homo sapiens c | 4.0 |
| 413374 | NM_001034 | Hs.75319 | ribonucleotide reductase M2 polypeptide | 4.0 |
| 413425 | F20956 | | gb:HSPD05390 HM3 Homo sapiens cDNA clone 032-X4-1 | 4.0 |
| 417655 | AA780791 | Hs.14014 | ESTs, Weakly similar to KIAA0973 protein [H. sapien | 4.0 |
| 424783 | AA913909 | Hs.153088 | TATA box binding protein (TBP)-associated factor, | 4.0 |
| 425024 | R39235 | Hs.12407 | ESTs | 4.0 |
| 445941 | AI267371 | Hs.172636 | ESTs | 4.0 |
| 448595 | AB014544 | Hs.21572 | KIAA0644 gene product | 4.0 |
| 453448 | AL036710 | Hs.209527 | ESTs | 4.0 |
| 458944 | N93227 | Hs.98403 | ESTs | 4.0 |
| 400284 | | | Estrogen receptor 1 | 4.0 |
| 441134 | W29092 | Hs.7678 | cellular retinoic acid-binding protein 1 | 4.0 |
| 408796 | AA688292 | Hs.118553 | ESTs | 4.0 |
| 408296 | AL117452 | Hs.44155 | DKFZP586G1517 protein | 4.0 |
| 438913 | AI380429 | Hs.172445 | ESTs | 4.0 |
| 402408 | | | 0 | 4.0 |
| 411630 | U42349 | Hs.71119 | Putative prostate cancer tumor suppressor | 4.0 |
| 450701 | H39960 | Hs.288467 | Homo sapiens cDNA FLJ12280 fis, clone MAMMA100174 | 4.0 |
| 439780 | AL109688 | | gb:Homo sapiens mRNA full length insert cDNA clone | 4.0 |
| 418301 | AW976201 | Hs.187618 | ESTs | 4.0 |
| 420077 | AW512260 | Hs.87767 | ESTs | 4.0 |
| 426572 | AB037783 | Hs.170623 | hypothetical protein FLJ11183 | 4.0 |
| 403721 | | | 0 | 4.0 |
| 411945 | AL033527 | Hs.92137 | v-myc avian myelocytomatosis viral oncogene homolo | 4.0 |
| 408684 | R61377 | Hs.12727 | hypothetical protein FLJ21610 | 4.0 |
| 414869 | AA157291 | Hs.72163 | ESTs | 4.0 |
| 437980 | R50393 | Hs.278436 | KIAA1474 protein | 4.0 |
| 451050 | AW937420 | Hs.69662 | ESTs | 4.0 |
| Table 1 shows 695 genes up-regulated in ovarian cancer compared to normal adult tissues. These were selected from 59680 probesets on the Affymetrix/Eos Hu03 GeneChip array such that the ratio of “average” | | | | |
| # ovarian cancer to “average” normal adult tissues was greater than or equal to 4.0. The “average” ovarian cancer level was set to the 90th percentile amongst 56 ovarian cancers obtained from the Garvan | | | | |
| # Institute for Molecular Research, Sydney, Australia. The “average” normal adult tissue level was set to the 90th percentile amongst 149 non- malignant tissues. In order to remove gene-specific background levels | | | | |
| # of non-specific hybridization, the 15th percentile value amongst the 149 non-malignant tissues was subtracted from both the numerator and the denominator before the ratio was evaluated. | | | | |

TABLE 2
499 UP-REGULATED GENES ENCODING EXTRACELLULAR/CELL SURFACE
PROTEINS, OVARIAN CANCER VERSUS NORMAL ADULT TISSUES

| | Exemplar | | | protein | ratio: |
|---|---|---|---|---|---|
| Primekey | Accession | UniGene | | structural | tumor |
| tissues | normal | ID | Title | domains | vs. |
| 415989 | AI267700 | Hs 111128 | ESTs | TM | 42.7 |
| 428579 | NM_005756 | Hs 184942 | G protein-coupled receptor 64 | TM | 30.5 |
| 428153 | AW513143 | Hs 98367 | similar to SRY-box containing gene 17 | TM | 30.1 |
| 436982 | AB018305 | Hs 5378 | spondin 1, (f-spondin) extracellular matrix | SS | 29.4 |
| 427585 | D31152 | Hs.179729 | collagen; type X; alpha 1 (Schmid metaphy | Clq, Collagen | 27.0 |
| 430691 | C14187 | Hs.103538 | ESTs | TM | 26.2 |
| 418007 | M13509 | Hs.83169 | Matrix metalloprotease 1 (interstitial collag | SS,, Peptidase_M10 | 20.6 |
| 400292 | AA250737 | Hs 72472 | BMPR-lb; bone morphogenetic protein rec | TM | 20.6 |
| 424086 | AI351010 | Hs.102267 | lysyl oxidase | Lysyl_oxidase | 17.7 |
| 424905 | NM_002497 | Hs.153704 | NIMA (never in mitosis gene a)-related km | pkise, pkinase | 17.4 |
| 427356 | AW023482 | Hs.97849 | ESTs | TM | 17.4 |
| 407638 | AJ404672 | Hs 288693 | EST | TM | 17.1 |
| 427469 | AA403084 | Hs.269347 | ESTs | TM | 17.0 |
| 438993 | AA828995 | | integrin; beta 8 | SS, integrin_B | 16.7 |
| 421155 | H87879 | Hs.102267 | lysyl oxidase | SS | 16.1 |
| 431989 | AW972870 | Hs 291069 | ESTs | SS | 15.9 |
| 428976 | AL037824 | Hs 194695 | ras homolog gene family, member 1 | ras | 15.1 |
| 416209 | AA236776 | Hs.79078 | MAD2 (mitotic arrest deficient, yeast, horn | TM | 15.0 |
| 413623 | AA825721 | Hs.246973 | ESTs | TM | 14.8 |
| 447350 | A1375572 | Hs.172634 | ESTs, HER4 (c-erb-B4) | SS, TM, Funn-like, pkinase | 14.2 |
| 428227 | AA321649 | Hs 2248 | INTERFERON-GAMMA INDUCED PRO | IL8 | 14.1 |
| 452461 | N78223 | Hs.108106 | transcription factor | G9a, PHD | 13.7 |
| 451106 | BE382701 | Hs 25960 | N-myc | Myc_N_term | 13.6 |
| 416208 | AW291168 | Hs.41295 | ESTs | TM | 13.5 |
| 452249 | BE394412 | Hs.61252 | ESTs | homeobox | 13.4 |
| 416566 | NM_003914 | Hs 79378 | cyclin A1 | cyclin | 12.8 |
| 416661 | AA634543 | Hs 79440 | IGF-II mRNA-binding protein 3 | TM | 12.6 |
| 431725 | X65724 | Hs 2839 | Norrie disease (pseudoglioma) | SS.Cys_knot | 12.3 |
| 458027 | L49054 | Hs.85195 | ESTs, Highly similar to t(3,5)(q25 1 ,p34) f | TM | 12.2 |
| 408460 | AA054726 | Hs.285574 | ESTs | TM | 12.2 |
| 415263 | AA948033 | Hs.130853 | ESTs | histone | 11.9 |
| 400298 | AA032279 | Hs.61635 | STEAP1 | TM | 11.8 |
| 421451 | AA291377 | Hs.50831 | ESTs | TM | 11 6 |
| 443715 | AI583187 | Hs.9700 | cyclin El | cyclin | 11.5 |
| 413472 | BE242870 | Hs.75379 | solute carrier family 1 (glial high affinity gl | TM.SDF | 11.5 |
| 410102 | AW248508 | Hs.279727 | ESTs, | SS | 11.4 |
| 408562 | A1436323 | Hs 31141 | Homo sapiens mRNA for KJAA1 568 prote | TM | 11.4 |
| 442353 | BE379594 | Hs 49136 | ESTs | TM | 11.3 |
| 427344 | NM_000869 | Hs 2142 | 5-hydroxytryptamme (serotonin) receptor 3 | TM, neur_chan | 11.2 |
| 453160 | A1263307 | Hs.146228 | ESTs | histone | 11.2 |
| 412723 | AA648459 | Hs 179912 | ESTs | TM | 11.1 |
| 400250 | | | 0 | Hist_deacetyl + F105 | 11.1 |
| 438167 | R28363 | Hs.24286 | ESTs | 7tm_1 | 11.1 |
| 434539 | AW748078 | Hs.214410 | ESTs | TM | 10.9 |
| 450375 | AA009647 | Hs 8850 | a dismtegrin and metalloproteinase domain | TM | 10.8 |
| 400289 | X07820 | Hs 2258 | Matrix Metalloproteinase 10 (Stromolysin 2 | SS.hemopexin | 10.8 |
| 446142 | A1754693 | Hs 145968 | ESTs | Cadhenn_C_term | 10.7 |
| 421285 | NM_000102 | Hs 1363 | cytochrome P450, subfamily XVII (steroid | TM, p450 | 10.6 |
| 433496 | AF064254 | Hs 49765 | VERY-LONG-CHAIN ACYL-COA SYNT | SS, TM | 10.6 |
| 418506 | AA084248 | Hs.85339 | G protein-coupled receptor 39 | TM | 10.5 |
| 433447 | U29195 | Hs.3281 | neuronal pentraxin 11 | SS | 10.4 |
| 414245 | BE148072 | Hs.75850 | WAS protein family, member 1 | TM | 10.3 |
| 426462 | U59111 | Hs.169993 | dermatan sulphate proteoglycan 3 | SS.LRRNT | 10.3 |
| 418601 | AA279490 | Hs 86368 | calmegin | SS | 10.3 |
| 415227 | AW821113 | Hs.72402 | ESTs | TM | 10.2 |
| 409269 | AA576953 | Hs 22972 | Homo sapiens cDNA FLJ13352 fis, clone O | TM | 10.1 |
| 426471 | M22440 | Hs.170009 | transforming growth factor, alpha | SS.EGF | 9.8 |
| 407881 | AW072003 | Hs.40968 | heparan sulfate (glucosamine) 3-O-sulfotran | SS | 9.7 |
| 445537 | AJ245671 | Hs 12844 | EGF-like-domain; multiple 6 | SS.EGF | 9.7 |
| 414972 | BE263782 | Hs.77695 | KIAA0008 gene product | TM | 9.4 |
| 435509 | AI458679 | Hs.181915 | ESTs | TM | 9.3 |
| 445413 | AA151342 | Hs.12677 | CG1- 147 protein | UPF0099 | 9.2 |
| 446999 | AA151520 | Hs 279525 | hypothetical protein PR02605 | TM | 9.1 |
| 414569 | AF109298 | Hs.118258 | Prostate cancer associated protein 1 | TM | 9.1 |
| 406687 | M31126 | Hs.272620 | pregnancy specific beta-1-glycoprotein 9 | hemopexin | 9.0 |
| 408908 | BE296227 | Hs.48915 | serine/threonine kinase 15 | pkise.TM | 9.0 |
| 451807 | W52854 | Hs.27099 | DKFZP564J0863 protein | TM | 8.8 |
| 420159 | AI572490 | Hs.99785 | ESTs | TM | 8.8 |
| 432677 | NM_004482 | Hs.278611 | UDP-N-acetyl-alpha-D-galactosamine.poly | TM, Ricin_B_lectm | 8.7 |
| 408829 | NM_006042 | Hs 48384 | heparan sulfate (glucosamine) 3-O-sulfotran | TM | 8.7 |
| 438885 | AI886558 | Hs.184987 | ESTs | TM | 8.7 |
| 447342 | AI199268 | Hs.19322 | ESTs; Weakly similar to !!!! ALU SUBFAM | TM | 8.6 |
| 437212 | A176502 1 | Hs.210775 | ESTs | UDPGT | 8.5 |
| 424717 | H03754 | Hs 152213 | wingless-type MMTV integration site fami | wnt | 8.4 |
| 450505 | NM_004572 | Hs 25051 | plakophilin 2 | TM | 8.4 |
| 436396 | A1683487 | Hs.299112 | Homo sapiens cDNA FLJ11441 fis, clone H | wnt | 8.3 |
| 425695 | NM_005401 | Hs.159238 | protein tyrosine phosphatase, non-receptor | Y_phosphatase | 8.3 |
| 447268 | A1370413 | Hs.36563 | Homo sapiens cDNA: FLJ22418 fis, clone | Ribosomal_S8 | 8.2 |
| 400195 | | | 0 | TM | 8.1 |
| 424906 | AI566086 | Hs 153716 | Homo sapiens mRNA for Hmob33 protein, | TM | 8.1 |
| 438202 | AW169287 | Hs.22588 | ESTs | TM | 8 1 |
| 439759 | AL359055 | Hs.67709 | Homo sapiens mRNA full length insert cDN | TM | 8.0 |
| 453102 | NM_007197 | Hs 31664 | frizzled (Drosophila) homolog 10 | TM, Fz, Frizzled | 8.0 |
| 424001 | W67883 | Hs 137476 | K1AA1051 protein | TM | 8.0 |
| 442655 | AW027457 | Hs 30323 | ESTs | TM | 7.8 |
| 445657 | AW612141 | Hs.279575 | ESTs | 7tm_1 | 7.8 |
| 426320 | W47595 | Hs.169300 | transforming growth factor, beta 2 | SSJGF-beta | 7.8 |
| 412170 | D16532 | Hs 73729 | very low density lipoprotein receptor | TM.ldl_recept_b, EGF | 7.6 |
| 436476 | AA326108 | Hs 53631 | ESTs | TM | 7.6 |
| 414132 | AI801235 | Hs.48480 | ESTs | TM | 7.6 |
| 437789 | A1581344 | Hs.127812 | ESTs, Weakly similar to AF141326 1 RNA | TM | 7.6 |
| 450192 | AA263143 | Hs.24596 | RAD51-interacting protein | TM | 7.6 |
| 408826 | AF216077 | Hs 48376 | Homo sapiens clone HB-2 mRNA sequence | TM | 7.5 |
| 413627 | BE182082 | Hs 246973 | ESTs | TM | 7.4 |
| 446293 | AI420213 | Hs.149722 | ESTs | LIM, homeobox | 7.4 |
| 409242 | AL080170 | Hs 51692 | DKFZP434C091 protein | TM, 7tm_1 | 7.3 |
| 450262 | AW409872 | Hs 271166 | ESTs, Moderately similar to ALU7_HUMA | TM | 7.3 |
| 451659 | BE379761 | Hs.14248 | ESTs, Weakly similar to ALU8_HUMAN A | TM | 7.3 |
| 444342 | NM_014398 | Hs.10887 | similar to lysosome-associated membrane g | TM | 7.2 |
| 429126 | AW172356 | Hs 99083 | ESTs | 7tm_1 | 7.1 |
| 421464 | AA291553 | Hs 190086 | ESTs | TM | 7.0 |
| 420362 | U79734 | Hs.97206 | huntingtin interacting protein 1 | TM | 7.0 |
| 444743 | AA045648 | Hs.11817 | nudix (nucleoside diphosphate linked moiet | TM | 7.0 |
| 415138 | C18356 | Hs.78045 | tissue factor pathway inhibitor 2 TFPI2 | Kunitz_BPTI.G-gamma | 6.9 |
| 429418 | AI381028 | Hs.99283 | ESTs | AAA | 6.9 |
| 409178 | BE393948 | Hs.50915 | Kallikrein 5 | SS, trypsin | 6.9 |
| 425905 | AB032959 | Hs.161700 | KIAA1133 protein | TM | 6.9 |
| 428532 | AF157326 | Hs.184786 | TBP-interacting protein | TM | 6.9 |
| 433426 | H69125 | Hs.133525 | ESTs | TM | 6.9 |
| 448674 | W31178 | Hs.154140 | ESTs | TM | 6.8 |
| 432415 | T16971 | Hs 289014 | ESTs | TM | 6.7 |
| 418203 | X54942 | Hs.83758 | CDC28 protein kinase 2 | TM | 6.6 |
| 438394 | BE379623 | Hs 27693 | CG1-124 protein | pro_isomerase | 6.6 |
| 452097 | AB002364 | Hs 27916 | ADAM-TS3; a dismtegrin-like and metal | Reprolysm | 6.6 |
| 453745 | AA952989 | Hs 63908 | Homo sapiens HSPC316 mRNA, partial cd | TGFb_propeptide | 6.6 |
| 423248 | AA380177 | Hs.125845 | ribulose-5-phosphate-3-epimerase | filament | 6.6 |
| 452281 | T93500 | Hs 28792 | ESTs | TGF-beta | 6.5 |
| 424620 | AA101043 | Hs 151254 | kallikrein 7 (chymotryptic; stratum corneum | SS.trypsin | 6.5 |
| 452594 | AU076405 | Hs.29981 | solute earner family 26 (sulfate transporter) | TM.Sulfate_transp | 6.5 |
| 434149 | Z43829 | Hs.19574 | ESTs, Weakly similar to katanin p80 subun | pkinase, fn3 | 6.5 |
| 425776 | U25128 | Hs 159499 | parathyroid hormone receptor 2 | TM,7tm_2 | 6.4 |
| 409517 | X90780 | Hs.54668 | troponin I, cardiac | Y_phosphatase | 6.4 |
| 432666 | AW204069 | Hs.129250 | ESTs, Weakly similar to unnamed protein p | TM | 6.4 |
| 448706 | AW291095 | Hs 21814 | class II cytokine receptor ZCYTOR7 | SS | 6.4 |
| 413582 | AW295647 | Hs.71331 | Homo sapiens cDNA FLJ21971 fis, clone | TM | 6.4 |
| 424153 | AA451737 | Hs.141496 | MAGE-like 2 | TM | 6.4 |
| 441081 | AI584019 | Hs.169006 | ESTs, Moderately similar to plakophilin 2b | PAX | 6.4 |
| 443539 | A1076182 | Hs.134074 | ESTs | TM | 6.4 |
| 418384 | AW149266 | Hs.25130 | ESTs | TM | 6.3 |
| 425371 | D49441 | Hs.155981 | mesothelin | SS | 6.3 |
| 449048 | Z45051 | Hs.22920 | similar to S68401 (cattle) glucose induced g | SS | 6.3 |
| 437117 | AL049256 | Hs 122593 | ESTs | TM | 6.3 |
| 453370 | AI470523 | Hs.182356 | ESTs, Moderately similar to translation init | ABC_tran | 6.3 |
| 426514 | BE616633 | Hs.301122 | bone morphogenetic protein 7 (osteogeric p | SS, TGF-beta | 6.3 |
| 452904 | AL157581 | Hs 30957 | Homo sapiens mRNA, cDNA DK.FZp434E | TM | 6.2 |
| 457030 | A1301740 | Hs 173381 | dihydropyrimidinase-like 2 | TM | 6.2 |
| 436281 | AW411194 | Hs.120051 | ESTs | TM | 6.1 |
| 415139 | AW975942 | Hs.48524 | ESTs | TM | 6.1 |
| 449448 | D60730 | Hs 57471 | ESTs | TM | 6.1 |
| 457979 | AA776655 | Hs.270942 | ESTs | TM | 6.1 |
| 422867 | L32137 | Hs.1584 | cartilage oligomeric matrix protein | SS, EGF, tsp_3 | 6.0 |
| 421502 | AF111856 | Hs.105039 | solute earner family 34 (sodium phosphate) | TM | 6.0 |
| 412733 | AA984472 | Hs.74554 | KIAA0080 protein | C2 | 6.0 |
| 422095 | A1868872 | Hs 288966 | ceruloplasmin (ferroxidase) | SS | 6.0 |
| 418845 | AA852985 | Hs.89232 | chromobox homolog 5 (Drosophila HP1 alp | Chromo_shadow | 6.0 |
| 410555 | U92649 | Hs.64311 | a disintegrin and metalloproteinase domain | TM,disintegrin, Reprolysin | 5.9 |
| 437099 | N77793 | Hs.48659 | ESTs, Highly similar to LMA1_HUMAN L | laminin_EGF | 5.9 |
| 453431 | AF094754 | Hs.32973 | glycine receptor, beta | TM.neur_chan | 5.9 |
| 417866 | AW067903 | Hs.82772 | “collagen, type XI, alpha 1” | TSPN, Collagen, COLF1 | 5.9 |
| 430291 | AV660345 | Hs 238126 | CGI-49 protein | TM | 5.9 |
| 405547 | #(NOCAT) | | 0 | TM, ABC_membrane | 5.9 |
| 435793 | AB037734 | Hs.4993 | ESTs | TM | 5.8 |
| 440138 | AB033023 | Hs.6982 | hypothetical protein FLJ10201 | TM | 5.8 |
| 425154 | NM_001851 | Hs 154850 | collagen, type IX, alpha 1 | SS, Collagen, TSPN | 5.7 |
| 419335 | AW960146 | Hs.284137 | Homo sapiens cDNA FLJ12888 fis, clone N | TM | 5.7 |
| 452971 | AI873878 | Hs 91789 | ESTs | TM | 5.7 |
| 428927 | AA441837 | Hs.90250 | ESTs | TM | 5.7 |
| 419247 | S65791 | Hs.89764 | fragile X mental retardation 1 | TM | 5.7 |
| 445640 | AW969626 | Hs 31704 | ESTs, Weakly similar to K1AA0227 [H. sap | TM | 5.7 |
| 447078 | AW885727 | Hs.301570 | ESTs | kazal | 5.6 |
| 421247 | BE391727 | Hs 102910 | general transcription factor IIH, polypeptid | TM | 5.6 |
| 432030 | AI908400 | Hs.143789 | ESTs | SS | 5.6 |
| 443270 | NM_004272 | Hs.9192 | Homer, neuronal immediate early gene, 1 B | TM | 5.5 |
| 411096 | U80034 | Hs.68583 | mitochondrial intermediate peptidase | Peptidase_M3 | 5.5 |
| 419558 | AW953679 | Hs 278394 | ESTs | SS | 5.5 |
| 427386 | AW836261 | Hs 177486 | amyloid beta (A4) precursor protein (protea | TM | 5.5 |
| 427961 | AW293165 | Hs.143134 | ESTs | TM | 5.5 |
| 407216 | N91773 | Hs 102267 | lysyl oxidase | TM | 5.5 |
| 413930 | M86153 | Hs 75618 | RAB11A, member RAS oncogene family | ras, TM | 5.5 |
| 414315 | Z24878 | | gb HSB65D052 STRATAGENE Human sk | TM | 5.5 |
| 441645 | AI222279 | Hs.201555 | ESTs | SS | 5.5 |
| 449318 | AW236021 | Hs.108788 | ESTs, Weakly similar to zeste [D. melanoga | TM | 5.4 |
| 441433 | AA933809 | Hs.42746 | ESTs | TM | 5.4 |
| 445495 | BE622641 | Hs.38489 | ESTs | I_LWEQ,ENTH | 5.4 |
| 410153 | BE311926 | Hs.15830 | Homo sapiens cDNA FLJ12691 fis, clone N | Glycos_transf_2 | 5.4 |
| 442611 | BE077155 | Hs.177537 | ESTs | TM | 5.4 |
| 452401 | NM_007115 | Hs 29352 | tumor necrosis factor, alpha-induced protein | Xlmk,CUB | 5.4 |
| 419948 | AB041035 | Hs 93847 | NADPH oxidase 4 | TM | 5.3 |
| 427718 | AI798680 | Hs.25933 | ESTs | histone | 5.3 |
| 453867 | AI929383 | Hs 108196 | HSPC037 protein | TM | 5.3 |
| 408298 | AI745325 | Hs 271923 | ESTs; Moderately similar to !!!! ALU SUB | Glycos_transf_2,DSPc | 5.3 |
| 448543 | AW897741 | Hs.21380 | Homo sapiens mRNA; cDNA DKFZp586P | TM | 5.3 |
| 433222 | AW514472 | Hs.238415 | ESTs, Moderately similar to ALU8_HUMA | TM | 5.3 |
| 449532 | W74653 | Hs 271593 | ESTs | TM | 5.3 |
| 452822 | X85689 | Hs 288617 | Homo sapiens cDNA-FLJ22621 fis, clone | TM, EGF, fn3 | 5.3 |
| 418379 | AA218940 | Hs 137516 | fidgetin-like 1 | AAA | 5.2 |
| 416530 | U62801 | Hs.79361 | kallikrein 6 (neurosin, zyme) | TM.trypsin | 5.2 |
| 413384 | NM_000401 | Hs.75334 | exostoses (multiple) 2 | TM | 5.2 |
| 445236 | AK001676 | Hs.12457 | hypothetical protein FLJ10814 | TM | 5.2 |
| 406367 | #(NOCAT) | | 0 | proteasome.trypsin | 5.2 |
| 442500 | AI819068 | Hs.209122 | ESTs | SS | 5.2 |
| 450101 | AV649989 | Hs 24385 | Human hbc647 mRNA sequence | TM | 5.2 |
| 419140 | AI982647 | Hs.2 15725 | ESTs | TM | 5.2 |
| 417791 | AW965339 | Hs.111471 | ESTs | Ald_Xan_dh_C | 5.1 |
| 437496 | AA452378 | Hs 170144 | Homo sapiens mRNA; cDNA DKFZp547Jl | TSPN, Folate_carrier | 5.1 |
| 418849 | AW474547 | Hs 53565 | ESTs, Weakly similar to B0491.1 [C. elegan | TM | 5.1 |
| 428093 | AW594506 | Hs.104830 | ESTs | TM | 5.1 |
| 408621 | AI970672 | Hs.46638 | chromosome 11 open reading frame 8; feta | TM | 5.1 |
| 418852 | BE537037 | Hs.273294 | hypothetical protein FLJ20069 | TM | 5.1 |
| 404939 | | | 0 | TM | 5.0 |
| 447020 | T27308 | Hs 16986 | hypothetical protein FLJ11046 | TM | 5.0 |
| 410824 | AW994813 | Hs.33264 | ESTs | TM | 5.0 |
| 417423 | AA197341 | Hs.111164 | ESTs | TM | 5.0 |
| 421477 | AI904743 | Hs 104650 | hypothetical protein FLJ10292 | TM | 5.0 |
| 443555 | N71710 | Hs 21398 | ESTs, Moderately similar to GNPI_HUMA | Glucosamine_iso | 5.0 |
| 424539 | L02911 | Hs 150402 | activin A receptor, type I | SS.Activin_recp.pkinase | 4.9 |
| 416565 | AW000960 | Hs.44970 | ESTs | TM | 4.9 |
| 431130 | NM_006103 | Hs 2719 | epididymis-specific; whey-acidic protein ty | SS | 4.9 |
| 408938 | AA059013 | Hs.22607 | ESTs | TM | 4.9 |
| 436754 | A1061288 | Hs.133437 | ESTs, Moderately similar to gonadotropin i | TM | 4.9 |
| 409049 | A1423132 | Hs.146343 | ESTs | TM | 4.9 |
| 458627 | AW088642 | Hs.97984 | ESTs; Weakly similar to WASP-family pro | TM | 4.8 |
| 418882 | NM_004996 | Hs.89433 | ATP-binding cassette, sub-family C (CFTR | TM.ABC_membrane | 4.8 |
| 422505 | AL120862 | Hs.124165 | ESTs; (HSA)PAP protein (programmed ce | TM | 4.8 |
| 428555 | NM_002214 | Hs 184908 | integrin, beta 8 | SS.integrin_B | 4.8 |
| 452909 | NM_015368 | Hs.30985 | pannexin 1 | TM | 4.8 |
| 449535 | W15267 | Hs.23672 | low density lipoprotein receptor-related pro | SS.ldl_recept_a.EGF | 4.8 |
| 452232 | AW020603 | Hs.271698 | ESTs | TM | 4.8 |
| 423161 | AL049227 | Hs 124776 | Homo sapiens mRNA; cDNA DKFZp564N | Cadherm_C_term | 4.7 |
| 428405 | Y00762 | Hs 2266 | cholinergic receptor, nicotinic, alpha polype | TM, neur_chan | 4.7 |
| 433330 | AW207084 | Hs 132816 | ESTs | TM | 4.7 |
| 443933 | AI091631 | Hs 135501 | Homo sapiens two pore potassium channel | TM | 4.7 |
| 440351 | AF030933 | Hs 7179 | RAD1 (S. pombe) homolog | TM | 4.7 |
| 426300 | U15979 | Hs.169228 | delta-like homolog (Drosophila) | TM,EGF | 4.7 |
| 453775 | NM_002916 | Hs.35120 | replication factor C (activator 1) 4 (37kD) | AAA, DEAD, hehcase_C | 4.7 |
| 429944 | R13949 | Hs.226440 | Homo sapiens clone 24881 mRNA sequenc | TM | 4.7 |
| 434988 | AI418055 | Hs.161160 | ESTs | TM | 4.6 |
| 406400 | #(NOCAT) | | 0 | trypsin.TM | 4.6 |
| 428301 | AW628666 | Hs.98440 | ESTs | TM | 4.6 |
| 446254 | BE179829 | Hs.179852 | Homo sapiens cDNA FLJ12832 fis, clone N | TM | 4.6 |
| 459574 | AI741122 | Hs.101810 | Homo sapiens cDNA FLJ14232 fis, clone N | TM | 4.6 |
| 409928 | AL137163 | Hs 57549 | hypothetical protein dJ473B4 | TM | 4.6 |
| 435244 | N77221 | Hs.187824 | ESTs | pkinase, fn3 | 4.6 |
| 404996 | #(NOCAT) | | 0 | Peptidase_Cl | 4.6 |
| 407905 | AW103655 | Hs.252905 | ESTs | SS.Ephrm | 4.6 |
| 441675 | AI914329 | Hs 5461 | ESTs | TM | 4.6 |
| 420276 | AA290938 | Hs 190561 | ESTs, Highly similar to mosaic protein LR1 | TM, fn3, ldl_recept_a | 4.5 |
| 422529 | AW015128 | Hs 256703 | ESTs | TM | 4.5 |
| 438018 | AK001160 | Hs.5999 | hypothetical protein FLJ10298 | TM | 4.5 |
| 457465 | AW301344 | Hs.195969 | ESTs | Pnbosyltran | 4.5 |
| 418848 | AI820961 | Hs.193465 | ESTs | TM.pkise | 4.5 |
| 447499 | AW262580 | Hs 147674 | KTAAI621 protein | TM | 4.5 |
| 432731 | R31178 | Hs 287820 | fibronectin 1 | SS | 4.5 |
| 434699 | AA643687 | Hs.149425 | Homo sapiens cDNA FLJ11980 fis, clone H | Nucleoside_tra2 | 4.4 |
| 427528 | AU077143 | Hs 179565 | minichromosome maintenance deficient (S. | TM | 4.4 |
| 409092 | AI735283 | Hs 172608 | ESTs | TM | 4.4 |
| 451389 | N73222 | Hs 21738 | KIAA 1008 protein | TM | 4.4 |
| 453331 | AI240665 | Hs.8895 | ESTs | TM | 4.4 |
| 448133 | AA723157 | Hs.73769 | folate receptor 1 (adult) | TM | 4.4 |
| 429597 | NM_003816 | Hs.2442 | a dismtegrin and metalloproteinase domain | TM | 4.4 |
| 453279 | AW893940 | Hs.59698 | ESTs | TM | 4.4 |
| 409459 | D86407 | Hs.54481 | low density lipoprotein receptor-related pro | TM, EGF, ldl_recept_a | 4.4 |
| 431708 | A1698136 | Hs.108873 | ESTs | TM | 4.4 |
| 433906 | AI167816 | Hs 43355 | ESTs | TM | 4.4 |
| 441423 | AI793299 | Hs.126877 | ESTs | TM | 4.4 |
| 446770 | AV660309 | Hs.154986 | ESTs, Weakly similar to AF137386 1 plasm | TM | 4.3 |
| 412078 | X69699 | Hs 73149 | paired box gene 8 | TM | 4.3 |
| 423123 | NM_012247 | Hs 124027 | SELENOPHOSPHATE SYNTHETASE; H | AIRS | 4.3 |
| 448390 | AL035414 | Hs.21068 | hypothetical protein | TM | 4.3 |
| 453628 | AW243307 | Hs 170187 | ESTs | TM | 4.3 |
| 452367 | U71207 | Hs.29279 | eyes absent (Drosophila) homolog 2 | TM | 4.3 |
| 413775 | AW409934 | Hs 75528 | nucleolar GTPase | MMR_HSR1 | 4.3 |
| 451592 | AI805416 | Hs 213897 | ESTs | TM | 4.3 |
| 419311 | AA689591 | | gb:nv66a12 s1 NCI_CGAP_GCB1 Homo s | TM | 4.2 |
| 452943 | BE247449 | Hs 31082 | hypothetical protein FLJ10525 | TM | 4.2 |
| 428679 | AA431765 | | gb:zw80c03 s1 Soares_testis_NHT Homo s | TM | 4.2 |
| 436209 | AW850417 | Hs.254020 | ESTs, Moderately similar to unnamed prote | TM | 4.2 |
| 406076 | AL390179 | Hs.137011 | Homo sapiens mRNA; cDNA DKFZp547P | TM | 4.2 |
| 428819 | AL135623 | Hs.193914 | KIAA0575 gene product | TM | 4.2 |
| 406671 | AA129547 | Hs.285754 | met proto-oncogene (hepatocyte growth fac | F-actin_cap_A | 4.2 |
| 431750 | AA514986 | Hs.283705 | ESTs | TM | 4.2 |
| 449554 | AA682382 | Hs.59982 | ESTs | TM | 4.2 |
| 409073 | AA063458 | | gb.zf71a07sl Soares_pineal gland N3HP | SEA | 4.1 |
| 433929 | AI375499 | Hs.27379 | ESTs | TM | 4.1 |
| 415457 | AW081710 | Hs 7369 | ESTs, Weakly similar to ALU1 HUMANA | TM | 4.1 |
| 444381 | BE387335 | Hs 283713 | ESTs | TM | 4.1 |
| 415539 | A1733881 | Hs.72472 | BMPR-Ib; bone morphogenetic protein rec | TM | 4.1 |
| 421515 | Y11339 | Hs 105352 | GalNAc alpha-2, 6-sialyltransferase I, long | TM | 4.1 |
| 453293 | AA382267 | Hs.10653 | ESTs | TM | 4.1 |
| 409564 | AA045857 | Hs.54943 | fracture callus 1 (rat) homolog | TM | 4.1 |
| 429628 | H09604 | Hs.13268 | ESTs | TM | 4.1 |
| 440452 | A1925136 | Hs.55150 | ESTs, Weakly similar to CAYP_HUMAN | TM | 4.1 |
| 443695 | AW204099 | Hs.112759 | ESTs, Weakly similar to AF126780 1 retina | TM | 4.1 |
| 425322 | U63630 | Hs.155637 | protein kinase, DNA-activated, catalytic po | TM | 4.1 |
| 417300 | AI765227 | Hs.55610 | solute earner family 30 (zinc transporter), m | TM | 4.1 |
| 417389 | BE260964 | Hs 82045 | Midkine (neurite growth-promoting factor 2 | SS, TM | 4.1 |
| 452834 | A1638627 | Hs.105685 | ESTs | kinesin | 4.1 |
| 428771 | AB028992 | Hs 193143 | KIAA1069 protein | PI-PLC-X.P1-PLC-Y | 4.0 |
| 412314 | AA825247 | Hs.250899 | heat shock factor binding protein 1 | TM | 4.0 |
| 436291 | BE568452 | Hs.5101 | ESTs; Highly similar to protein regulating c | TM | 4.0 |
| 450654 | AJ245587 | Hs 25275 | Kruppel-type zinc finger protein | KRAB | 4.0 |
| 409365 | AA702376 | Hs.226440 | Homo sapiens clone 24881 mRNA sequenc | TM | 4.0 |
| 413374 | NM_001034 | Hs.75319 | ribonucleotide reductase M2 polypeptide | ribonuc_red | 4.0 |
| 417655 | AA780791 | Hs 14014 | ESTs, Weakly similar to KIAA0973 protein | TM | 4.0 |
| 445941 | A1267371 | Hs 172636 | ESTs | TM,lectm_c | 4.0 |
| 441134 | W29092 | Hs.7678 | cellular retinoic acid-binding protein 1 | lipocalin | 4.0 |
| 411630 | U42349 | Hs.71119 | Putative prostate cancer tumor suppressor | TM | 4.0 |
| 418301 | AW976201 | Hs.187618 | ESTs | TM | 4.0 |
| 411945 | AL033527 | Hs 92137 | v-myc avian myelocytomatosis viral oncog | TGF-beta, Myc_N_term | 4.0 |
| 408684 | R61377 | Hs 12727 | hypothetical protein FLJ21610 | TM | 4.0 |
| 414869 | AA157291 | Hs.72163 | ESTs | TM | 4.0 |
| 420281 | AI623693 | Hs.191533 | ESTs | Cation_efflux | 3.9 |
| 416658 | U03272 | Hs.79432 | fibrillin 2 (congenital contractural arachnod | EGF.TB | 3.9 |
| 411274 | NM_002776 | Hs.69423 | kallikrein 10 | trypsin, TM | 3.9 |
| 437222 | AL117588 | Hs.299963 | ESTs | TM | 3.9 |
| 431958 | X63629 | Hs.2877 | Cadherin 3, P-cadherin (placental) | TM, cadherin, | 3.9 |
| 430634 | AI860651 | Hs 26685 | ESTs | TM | 3.9 |
| 415716 | N59294 | Hs.301141 | Homo sapiens cDNA FLJ11689 fis, clone H | NAP_family | 3.9 |
| 420179 | N74530 | Hs 21168 | ESTs | TM | 3.8 |
| 451250 | AA491275 | Hs 236940 | Homo sapiens cDNA FLJ12542 fis, clone N | TM | 3.8 |
| 429496 | AA453800 | Hs.192793 | ESTs | TM | 3.8 |
| 421764 | AI681535 | Hs.99342 | ESTs, Weakly similar to KCC1_HUMAN C | TM | 3.8 |
| 447197 | R36075 | | gb:yh88b01.sl Scares placenta Nb2HP Horn | TM, SDF | 3.8 |
| 422939 | AW394055 | Hs.98427 | ESTs | TM | 3.8 |
| 414737 | AI160386 | Hs.125087 | ESTs | TM | 3.8 |
| 411773 | NM_006799 | Hs.72026 | protease, serine, 21 (testisin) | SS.trypsin | 3.8 |
| 425247 | NM_005940 | Hs.155324 | matrix metalloproteinase 11 (stromelysin 3) | SS, Peptidase_M10 | 3.7 |
| 424433 | H04607 | Hs 9218 | ESTs | TM | 3.7 |
| 431846 | BE019924 | Hs.271580 | Uroplakin IB | TM_, transmembrane4 | 3.7 |
| 407792 | AI077715 | Hs.39384 | putative secreted ligand homologous to fjx1 | SS | 3.7 |
| 417531 | NM_003157 | Hs.1087 | serine/threonine kinase 2 | pkise, pkinase | 3.7 |
| 434836 | AA651629 | Hs.118088 | ESTs | TM | 3.7 |
| 439810 | AL109710 | Hs 85568 | EST | TM | 3.7 |
| 418693 | AI750878 | Hs.87409 | thrombospondin 1 | SS, EGF, TSPN | 3.7 |
| 407864 | AF069291 | Hs.40539 | chromosome 8 open reading frame 1 | TM | 3.7 |
| 436304 | AA339622 | Hs 108887 | ESTs | TM | 3.7 |
| 452259 | AA317439 | Hs.28707 | signal sequence receptor, gamma (transloco | TM | 3.7 |
| 453468 | W00712 | Hs.32990 | DK.FZP566F084 protein | TM | 3.6 |
| 428943 | AW086180 | Hs.37636 | ESTs, Weakly similar to KIAA1392 protein | TM | 3.6 |
| 411402 | BE297855 | Hs 69855 | NRAS-related gene | CSD, ras, CSD | 3.6 |
| 425176 | AW015644 | Hs.301430 | ESTs, Moderately similar to TEF1_HUMA | TM | 3.6 |
| 400296 | AA305627 | Hs.139336 | ATP-binding cassette, sub-family C (CFTR | ABC_tran | 3.6 |
| 407340 | AA810168 | Hs.232119 | ESTs | TM | 3.6 |
| 418524 | AA300576 | Hs.85769 | acidic 82 kDa protein mRNA | TM | 3.6 |
| 438279 | AA805166 | Hs.165165 | ESTs, Moderately similar to ALU8_HUMA | TM | 3.6 |
| 439453 | BE264974 | Hs 6566 | thyroid hormone receptor interactor 13 | AAA.AAA | 3.6 |
| 441111 | A1806867 | Hs.126594 | ESTs | TM | 3.6 |
| 451806 | NM_003729 | Hs.27076 | RNA 3′-terminal phosphate cyclase | TM | 3.6 |
| 409542 | AA503020 | Hs.36563 | ESTs | Ribosomal_S8 | 3.6 |
| 425441 | AA449644 | Hs.193063 | Homo sapiens cDNA FLJ14201 fis, clone N | Aa_trans | 3.6 |
| 428137 | AA421792 | Hs 170999 | ESTs | AAA | 3.6 |
| 433692 | AI805860 | Hs.208675 | ESTs, Weakly similar to neuronal thread pr | TM | 3.6 |
| 438689 | AW129261 | Hs.250565 | ESTs | TM | 3.6 |
| 443341 | AW631480 | Hs 8688 | ESTs | TM | 3.6 |
| 446261 | AA313893 | Hs 13399 | hypothetical protein FLJ12615 similar to m | ATP-synt_D, PH | 3.6 |
| 414343 | AL036166 | Hs.75914 | coated vesicle membrane protein | TM | 3.5 |
| 414812 | X72755 | Hs 77367 | monokine induced by gamma interferon | SS, IL8 | 3.5 |
| 410361 | BE391804 | Hs 62661 | guanylate binding protein 1, interferon-indu | TM | 3.5 |
| 415786 | AW419196 | Hs 257924 | ESTs | TM | 3.5 |
| 427177 | AB006537 | Hs 173880 | interleukin 1 receptor accessory protein | TM.ig | 3.5 |
| 427687 | AW003867 | Hs 112403 | ESTs | 7tm_1 | 3.5 |
| 444619 | BE538082 | Hs.8172 | ESTs | TM | 3.5 |
| 447336 | AW139383 | Hs.245437 | ESTs | AhpC-TSA | 3.5 |
| 412519 | AA196241 | Hs.73980 | troponin T1, skeletal, slow | TM | 3.5 |
| 418792 | AB037805 | Hs.88442 | K1AA1384 protein | TM | 3.5 |
| 408031 | AA081395 | Hs.42173 | Homo sapiens cDNA FLJ10366 fis, clone N | TM | 3.5 |
| 416892 | L24498 | Hs.80409 | growth arrest and DNA-damage-inducible, | TM | 3.5 |
| 418793 | AW382987 | Hs 88474 | prostaglandin-endoperoxide synthase 1 (pro | EGF | 3.5 |
| 448089 | AI467945 | Hs.173696 | ESTs | SS | 3.5 |
| 422278 | AF072873 | Hs 114218 | ESTs | TM, Fz, Frizzled | 3.5 |
| 442133 | AW874138 | Hs.129017 | ESTs | TM | 3.5 |
| 410908 | AA121686 | Hs.10592 | ESTs | GTP_EFTU | 3.5 |
| 452198 | AI097560 | Hs.61210 | ESTs | TM | 3.5 |
| 408730 | AV660717 | Hs.47I44 | DKFZP586N08 19 protein | pkinase | 3.4 |
| 436488 | BE620909 | Hs 261023 | hypothetical protein FLJ20958 | TM | 3.4 |
| 409745 | AA077391 | | gb′7B14E12 Chromosome 7 Fetal Brain cD | TM | 3.4 |
| 445870 | AW410053 | Hs 13406 | syntaxin 18 | TM | 3.4 |
| 451743 | AW074266 | Hs.23071 | ESTs | TM | 3.4 |
| 407846 | AA426202 | Hs.40403 | Cbp/p300-mteracting transactivator, with G | TM | 3.4 |
| 432350 | NM_005865 | Hs 274407 | protease, serine, 16 (thymus) | SS | 3.4 |
| 412848 | AA121514 | Hs.70832 | ESTs | TM | 3.4 |
| 413625 | AW451103 | Hs.71371 | ESTs | filament | 3.4 |
| 417801 | AA417383 | Hs 82582 | integrin, beta-like 1 (with EGF-like repeat d | SS | 3.4 |
| 422972 | N59319 | Hs 145404 | ESTs | TM | 3.4 |
| 429170 | NM_001394 | Hs.2359 | dual specificity phosphatase 4; MAP kinas | DSPc, Rhodanese | 3.4 |
| 450377 | AB033091 | Hs 24936 | ESTs | TM | 3.4 |
| 443475 | AI066470 | Hs.134482 | ESTs | TM | 3.4 |
| 419452 | U33635 | Hs.90572 | PTK.7 protein tyrosine kinase 7 | TM, pkise, ig, SRF-TF | 3.4 |
| 409744 | AW675258 | Hs.56265 | Homo sapiens mRNA; cDNA DKFZp586P | TM | 3.4 |
| 422789 | AK001113 | Hs.120842 | hypothetical protein FLJ10251 | TM | 3.4 |
| 404440 | #(NOCAT) | | 0 | TM.neur_chan | 3.4 |
| 417412 | X16896 | Hs.82112 | interleukin 1 receptor, type I | SS, TIR, ig | 3.4 |
| 411828 | AW161449 | Hs.72290 | wingless-type MMTV integration site fami | wnt | 3.4 |
| 417177 | NM_004458 | Hs.81452 | fatty-acid-Coenzyme A ligase, long-chain 4 | SS | 3.4 |
| 421013 | M62397 | Hs.1345 | mutated in colorectal cancers | TM | 3.4 |
| 427072 | H38046 | | gb yp58c10.r1 Scares fetal liver spleen INF | Ribosomal_L22e | 3.4 |
| 433703 | AA210863 | Hs.3532 | nemo-like kinase | pkinase | 3.4 |
| 434294 | AJ271379 | Hs.21175 | ESTs | TM | 3.4 |
| 444188 | AI393165 | Hs.19175 | ESTs | TM | 3.4 |
| 446109 | N67953 | Hs.145920 | ESTs | TM | 3.4 |
| 400881 | | | 0 | Asparaginase_2 | 3.3 |
| 450236 | AW162998 | Hs.24684 | KIAA1376 protein | TM | 3.3 |
| 418836 | AI655499 | Hs 161712 | ESTs | TM | 3.3 |
| 437951 | T34530 | Hs.4210 | Homo sapiens cDNA FLJ13069 fis, clone N | TM | 3.3 |
| 446896 | T15767 | Hs.22452 | Homo sapiens cDNA. FLJ21084 fis, clone | TM | 3.3 |
| 430687 | BE274217 | Hs 249247 | heterogeneous nuclear protein similar to rat | rrm | 3.3 |
| 410060 | NM_001448 | Hs 58367 | glypican-4 | SS | 3.3 |
| 419546 | AA244199 | | gb:nc06c05.sl NCl_CGAP_PrI Homo sapi | TM | 3.3 |
| 429609 | AF002246 | Hs.210863 | cell adhesion molecule with homology to L | TM, fn3, ig | 3.3 |
| 413289 | AA128061 | Hs 114992 | ESTs | TM | 3.3 |
| 440006 | AK000517 | Hs.6844 | hypothetical protein FLJ20510 | TM | 3.3 |
| 401435 | #(NOCAT) | | 0 | TM | 3.3 |
| 420072 | AW961196 | Hs.207725 | ESTs | TM | 3.3 |
| 421426 | AA291101 | Hs.33020 | Homo sapiens cDNA FLJ20434 fis, clone K | TM | 3.3 |
| 425851 | NM_001490 | Hs.159642 | glucosaminyl (N-acetyl) transferase 1 , core | SS | 3.3 |
| 443295 | AI049783 | Hs 241284 | ESTs | TM | 3.2 |
| 453116 | AI276680 | Hs.146086 | ESTs | Ribosomal_L5_C | 3.2 |
| 456546 | AI690321 | Hs.203845 | ESTs, Weakly similar to TWIK-related acid | TM | 3.2 |
| 430016 | NM_004736 | Hs 227656 | xenotropic and polytropic retrovirus recepto | TM | 3.2 |
| 418281 | U09550 | Hs 1154 | oviductal glycoprotein 1, 120kD (mucin 9, | asp, Glyco_hydro_18 | 3.2 |
| 433800 | A1034361 | Hs.135150 | lung type-I cell membrane-associated glyco | TM | 3.2 |
| 425159 | NM_004341 | Hs.154868 | carbamoyl-phosphate synthetase 2, aspartat | TM | 3.2 |
| 428882 | AA436915 | Hs.131748 | ESTs, Moderately similar to ALU7_HUMA | Carb_anhydrase | 3.2 |
| 409533 | AW969543 | Hs 21291 | mitogen-activated protein kinase kinase km | TM | 3.2 |
| 411248 | AA551538 | Hs 69321 | KIAA1359 protein | TM | 3.2 |
| 421379 | Y15221 | Hs.103982 | small inducible cytokine subfamily B (Cys- | SS, IL8 | 3.2 |
| 430259 | BE550182 | Hs 127826 | RalGEF-like protein 3, mouse homolog | TM | 3.2 |
| 414945 | BE076358 | Hs.77667 | lymphocyte antigen 6 complex, locus E | SS | 3.2 |
| 444471 | AB020684 | Hs 11217 | KIAA0877 protein | TM | 3.2 |
| 421674 | T10707 | Hs.296355 | neuronal PAS domain protein 2 | Ribosomal_L31e | 3.2 |
| 434163 | AW974720 | Hs 25206 | ESTs | TM | 3.2 |
| 421991 | NMJM4918 | Hs.110488 | KIAA0990 protein | SS | 3.2 |
| 409589 | AW439900 | Hs.256914 | ESTs | TM | 3.2 |
| 414147 | BE091634 | | gb:IL2-BT0731-240400-069-C03BT0731 | TM | 3.2 |
| 414661 | T97401 | Hs.21929 | ESTs | TM | 3.2 |
| 437537 | AA758974 | Hs 121417 | ESTs, Weakly similar to unnamed protein p | TM | 3.2 |
| 439702 | AW085525 | Hs.134182 | ESTs | A2M | 3.1 |
| 420552 | AK000492 | Hs 98806 | hypothetical protein | TM | 3.1 |
| 441028 | AI333660 | Hs.17558 | ESTs | ICE_p20, CARD | 3.1 |
| 425264 | AA353953 | Hs 20369 | ESTs, Weakly similar to gonadotropin indu | TM | 3.1 |
| 422109 | S73265 | Hs.1473 | gastrin-releasing peptide | SS, Bombesin | 3.1 |
| 441859 | AW194364 | Hs 128022 | ESTs, Weakly similar to FIG. 1 MOUSE FIG | TM | 3.1 |
| 415451 | H19415 | Hs 268720 | ESTs, Moderately similar to ALU1_HUMA | SS.Ephrm | 3.1 |
| 447866 | AW444754 | Hs.211517 | ESTs | homeobox | 3.1 |
| 419978 | NM_001454 | Hs.93974 | forkhead box J1 | Fork_head | 3.1 |
| 446219 | AI287344 | Hs 149827 | ESTs | M1P | 3.1 |
| 448428 | AF282874 | Hs 21201 | nectin 3; DKPZP566B0846 protein | TM, ig | 3.1 |
| 407615 | AW753085 | | gb:PM1-CT0247-151299-005-a03 CT0247 | TM | 3.1 |
| 410518 | AW976443 | Hs.285655 | ESTs | RasGEF, PH, RhoGEF | 3.1 |
| 418396 | A1765805 | Hs.26691 | ESTs | TM | 3.1 |
| 427855 | R61253 | Hs 98265 | ESTs | TM | 3.1 |
| 429272 | W25140 | Hs.110667 | ESTs | TM | 3.1 |
| 450171 | AL133661 | Hs.24583 | hypothetical protein DKFZp434C0328 | TM | 3.1 |
| 414774 | X02419 | Hs 77274 | plasminogen activator, urokinase | SS, kringle, trypsin | 3.1 |
| 422363 | T55979 | Hs.115474 | replication factor C (activator 1) 3 (38kD) | TM | 3.1 |
| 420062 | AW411096 | Hs.94785 | hypothetical protein LOC57163 | TM | 3 1 |
| 428698 | AA852773 | Hs 297939 | ESTs; Weakly similar to neogenin [H. sapie | TM | 3.1 |
| 427051 | BE178110 | Hs.173374 | ESTs | TM | 3.1 |
| 428242 | H55709 | Hs 2250 | leukemia inhibitory factor (cholinergic diffe | SS | 3.1 |
| 452906 | BE207039 | Hs.75621 | serine (or cysteine) proteinase inhibitor, cla | TM | 3.1 |
| 429419 | AB023226 | Hs 202276 | K.IA A 1009 protein | TM | 3.1 |
| 417517 | AF001176 | Hs 82238 | POP4 (processing of precursor , S. cerevisia | TM | 3 1 |
| 406137 | #(NOCAT) | | 0 | TM | 3.1 |
| 424800 | AL035588 | Hs.153203 | MyoD family inhibitor | TM | 3.1 |
| 410252 | AW821182 | Hs.61418 | microfibrillar-associated protein 1 | TM | 3.1 |
| 420392 | AI242930 | Hs.97393 | KIAA0328 protein | SS | 3.1 |
| 423629 | AW021173 | Hs.18612 | Homo sapiens cDNA: FLJ21909 fis, clone | voltage_CLC, CBS | 3.1 |
| 429334 | D63078 | Hs 186180 | Homo sapiens cDNA. FLJ23038 fis, clone | Glyco_hydro_2 | 3.1 |
| 449802 | AW901804 | Hs 23984 | hypothetical protein FU20147 | TM | 3 1 |
| 450506 | NM_004460 | Hs 418 | fibroblast activation protein; alpha | SS.Peptidase_S9 | 3.0 |
| 433849 | BE465884 | Hs.280728 | ESTs | TM | 3.0 |
| 411984 | NM_005419 | Hs.72988 | signal transducer and activator of transcript | SH2, STAT | 3.0 |
| 422530 | AW972300 | Hs 118110 | bone marrow stromal cell antigen 2 | TM | 3.0 |
| 422128 | AW881145 | | gb.QVO-OT0033-010400-182-a07 OT0033 | TM | 3.0 |
| 409757 | NM_001898 | Hs.123114 | cystatin SN | SS, cystatin | 3.0 |
| 418727 | AA227609 | Hs 94834 | ESTs | TM | 3.0 |
| 422244 | Y08890 | Hs.113503 | karyopherin (importin) beta 3 | TM | 3.0 |
| 456844 | AI264155 | Hs.152981 | CDP-diacylglycerol synthase (phosphatidat | TM | 3.0 |
| 432358 | AI093491 | Hs.72830 | ESTs | SS | 3.0 |
| 416896 | AI752862 | Hs.5638 | KIAA1572 protein | BTB | 3.0 |
| 447312 | A1434345 | Hs.36908 | activating transcription factor 1 | TM | 3.0 |
| 445021 | AK002025 | Hs.12251 | Homo sapiens cDNA FLJ1 1 163 fis, clone P | TM | 3.0 |
| 422611 | AA158177 | Hs.118722 | fucosyltransferase 8 (alpha (1,6) fucosyltran | SS | 3.0 |
| 453597 | BE281130 | Hs 33713 | myo-mositol 1-phosphate synthase Al | TM | 3.0 |
| 401197 | #(NOCAT) | | 0 | arf.Ets | 3.0 |
| 403000 | BE247275 | Hs.151787 | U5 snRNP-specific protein, 116 kD | TM | 3.0 |
| 410008 | AA079552 | | gb:zm20h12.s1 Stratagene pancreas (93720 | TM, FG-GAP | 3.0 |
| 413268 | AL039079 | Hs.75256 | regulator of G-protein signalling 1 | RGS | 3.0 |
| 414080 | AA135257 | Hs 47783 | ESTs, Weakly similar to T12540 hypotheti | TM | 3.0 |
| 426882 | AA393108 | Hs.97365 | ESTs | TM | 3.0 |
| 427651 | AW405731 | Hs.18498 | Homo sapiens cDNA FLJ12277 fis, clone M | TM | 3.0 |
| 439444 | A1277652 | Hs.54578 | ESTs | TM | 3.0 |
| 433001 | AF217513 | Hs.279905 | clone HQ0310 PRO0310pl | TM | 3.0 |
| 444895 | AI674383 | Hs 301192 | EST cluster (not in UniGene) | TM.ASC | 3.0 |
| 441962 | AW972542 | Hs.289008 | Homo sapiens cDNA: FLJ21814 fis, clone | TM | 3.0 |
| 414725 | AA769791 | Hs 120355 | Homo sapiens cDNA FLJ13148 fis, clone N | TM, 7tm_1 | 3.0 |
| 434241 | AP119913 | Hs 283607 | hypothetical protein PRO3077 | SS | 3.0 |
| 424962 | NM_012288 | Hs.153954 | TRAM-like protein | TM | 3.0 |
| 411987 | AA375975 | Hs.183380 | ESTs, Moderately similar to ALU7_HUMA | TM | 3.0 |
| 421977 | W94197 | Hs.110165 | ribosomal protein L26 homolog | TM | 3.0 |
| 436481 | AA379597 | Hs.5199 | HSPC150 protein similar to ubiquitin-conju | TM | 3.0 |
| 407872 | AB039723 | Hs.40735 | frizzled (Drosophila) homolog 3 | TM, 7tm_2, Fz, Frizzled | 3.0 |
| 442577 | AA292998 | Hs.163900 | ESTs | TM | 3.0 |
| 416120 | H46739 | | gb:yo14h02.sl Scares adult brain N2b5HB5 | TM | 3.0 |
| 443775 | AF291664 | Hs.204732 | matrix metalloproteinase 26 | TM.Peptidase_M10, 7tm_1 | 3.0 |
| 414664 | AA587775 | Hs 66295 | Homo sapiens HSPC3 11 mRNA, partial cd | TM | 3.0 |
| 457590 | AI612809 | Hs.5378 | spondin 1, (f-spondin) extracellular matrix | SS | 3.0 |
| 418946 | AI798841 | Hs.132103 | ESTs | TM | 3.0 |
| 457940 | AL360159 | Hs 30445 | Homo sapiens mRNA full length insert cDN | TM, SPRY, 7tm_1 | 3.0 |
| Table 2 shows 499 genes up-regulated in ovarian cancer compared to normal adult tissues that are likely to be extracellular or cell-surface proteins. These were selected as for Table 1, except that the ratio was greater | | | | | |
| # than or equal to 3.0, and the predicted protein contained a structural domain that is indicative of extracellular localization (e.g. ig, fh3, egf, 7tm domains). The predicted protein domains are noted. | | | | | |

TABLE 3
92 UP-REGULATED GENES, MUCINOUS OVARIAN
CANCER VERSUS NORMAL ADULT TISSUES

| | Exemplar | | | protein | ratio: |
|---|---|---|---|---|---|
| Primekey | Accession | UniGene | | structural | tumor |
| tissues | normal | ID | Title | domains | vs. |
| 430691 | C14187 | Hs.103538 | ESTs | | 34.9 |
| 432938 | T27013 | Hs.3132 | steroidogenic acute regulatory protein | START | 28.0 |
| 418007 | M13509 | Hs.83169 | Matrix metalloprotease 1 (interstitial collag | SS, Peptidase_M10 | 22.3 |
| 451181 | A1796330 | Hs 207461 | ESTs | | 10.8 |
| 452838 | U65011 | Hs 30743 | Preferentially expressed antigen in melanom | | 10.0 |
| 407638 | AJ404672 | Hs 288693 | EST | | 9.3 |
| 450159 | A1702416 | Hs 200771 | ESTs, Weakly similar to CAN2_HUMAN | | 9.2 |
| 426890 | AA393167 | Hs.41294 | ESTs | | 9.1 |
| 421155 | H87879 | Hs.102267 | lysyl oxidase | SS, Lysyl_oxidase | 8.9 |
| 437099 | N77793 | Hs.48659 | ESTs, Highly similar to LMA1 HUMAN L | laminin_EGF | 7.6 |
| 453866 | AW291498 | Hs.250557 | ESTs | | 7.6 |
| 435496 | AW840171 | Hs.265398 | ESTs, Weakly similar to transformation-rel | | 7.4 |
| 418738 | AW388633 | Hs 6682 | solute carrier family 7, member 11 | | 7.2 |
| 431956 | AK002032 | Hs.272245 | Homo sapiens cDNA FLJ11170 fis, clone P | RA | 7.0 |
| 449579 | AW207260 | Hs.134014 | prostate cancer associated protein 6 | | 6.7 |
| 424586 | NM_003401 | Hs.150930 | X-ray repair complementing defective repa | | 6.7 |
| 445891 | AW391342 | Hs.199460 | ESTs | | 6.2 |
| 424717 | H03754 | Hs.152213 | wingless-type MMTV integration site fami | wnt | 6.1 |
| 452705 | H49805 | Hs.246005 | ESTs | | 6.1 |
| 421285 | NM_000102 | Hs.1363 | cytochrome P450, subfamily XVII (steroid | TM, p450 | 5.5 |
| 408562 | AI436323 | Hs.31141 | Homo sapiens mRNA for KIAA1568 prote | | 5.3 |
| 420159 | AI572490 | Hs.99785 | ESTs | | 5.3 |
| 451105 | AI761324 | | gb:wi60b1.x1 NCI_CGAP_Col6 Homo s | | 5.2 |
| 409049 | AI423132 | Hs.146343 | ESTs | | 5.0 |
| 448674 | W31178 | Hs.154140 | ESTs | TM | 5.0 |
| 423811 | AW299598 | Hs 50895 | homeo box C4 | | 4.9 |
| 427469 | AA403084 | Hs.269347 | ESTs | | 4.9 |
| 447033 | AI357412 | Hs.157601 | EST - not in UniGene | PH | 4.9 |
| 424433 | H04607 | Hs 9218 | ESTs | | 4.9 |
| 448811 | AI590371 | Hs.174759 | ESTs | TM | 4.8 |
| 444330 | AI597655 | Hs.49265 | ESTs | | 4.8 |
| 409041 | AB033025 | Hs.50081 | KIAA1199 protein | | 4.7 |
| 418735 | N48769 | Hs.44609 | ESTs | | 4.5 |
| 416661 | AA634543 | Hs.79440 | IGF-II mRNA-binding protein 3 | KH-domain | 4.5 |
| 430073 | U86136 | Hs.232070 | telomerase-associated protein 1 | WD40 | 4.4 |
| 407881 | AW072003 | Hs.40968 | heparan sulfate (glucosamine) 3-O-sulfotran | SS | 4.4 |
| 422260 | AA315993 | Hs.105484 | ESTs; Weakly similar to LITHOSTATHIN | | 4.4 |
| 421110 | AJ250717 | Hs.1355 | cathepsin E | SS, asp | 4.3 |
| 445676 | AI247763 | Hs.16928 | ESTs | | 4.2 |
| 430704 | AW813091 | | gb:RC3-ST0186-240400-111-d07 ST0186 | Epimerase | 3.8 |
| 414569 | AP109298 | Hs 118258 | Prostate cancer associated protein 1 | TM | 3.8 |
| 438078 | AI016377 | Hs.131693 | ESTs | | 3.7 |
| 434032 | AW009951 | Hs.206892 | ESTs | | 3.7 |
| 445657 | AW612141 | Hs.279575 | ESTs | 7tm_1 | 3.6 |
| 439759 | AL359055 | Hs 67709 | Homo sapiens mRNA full length insert cDN | | 3.5 |
| 455666 | BE065813 | | gb RC2-BT0318-110100-012-a08 BT0318 | | 3.5 |
| 448844 | AI581519 | Hs.177164 | ESTs | | 3.5 |
| 449048 | Z45051 | Hs.22920 | similar to S68401 (cattle) glucose induced g | SS | 3.5 |
| 438018 | AK001160 | Hs.5999 | hypothetical protein FLJ10298 | TM | 3.4 |
| 458123 | AW892676 | | gb:CM3-NN0004-280300-131-cl2NN0004 | | 3.4 |
| 407385 | AA610150 | Hs 272072 | ESTs, Moderately similar to ALU7_HUMA | | 3.4 |
| 424894 | H83520 | Hs.153678 | reproduction 8 | SS, UBX | 3.3 |
| 424639 | AI917494 | Hs.131329 | ESTs | | 3.3 |
| 414083 | AL121282 | Hs.257786 | ESTs | | 3.2 |
| 426471 | M22440 | Hs.170009 | transforming growth factor, alpha | SS, EGF | 3.2 |
| 428927 | AA441837 | Hs 90250 | ESTs | | 3. |
| 406129 | #(NOCAT) | | 0 | TM, cNMP_binding | 3. |
| 452699 | AW295390 | Hs.213062 | ESTs | | 3. |
| 425842 | A1587490 | Hs.159623 | NK-2 (Drosophila) homolog B | homeobox | 3. |
| 428976 | AL037824 | Hs.194695 | ras homolog gene family, member I | ras | 3. |
| 436396 | AI683487 | Hs 299112 | Homo sapiens cDNA FLJ11441 fis, clone H | wnt | 3.0 |
| 454077 | AC005952 | Hs 37062 | insulin-like 3 (Leydig cell) | SS, Insulin, pkinase | 3.0 |
| 404253 | #(NOCAT) | | 0 | histone | 2.9 |
| 452461 | N78223 | Hs 108106 | transcription factor | G9a, PHD | 2.9 |
| 429597 | NM_003816 | Hs.2442 | a disintegrin and metalloproteinase domain | TM | 2.9 |
| 413289 | AA128061 | Hs.114992 | ESTs | | 2.9 |
| 429703 | T93154 | Hs 28705 | ESTs | | 2.9 |
| 407829 | AA045084 | Hs.29725 | Homo sapiens cDNA FLJ13197 fis, clone N | | 2.8 |
| 424796 | AW298244 | Hs.293507 | ESTs | | 2.8 |
| 424086 | AI351010 | Hs.102267 | lysyl oxidase | Lysyl_oxidase | 2.8 |
| 408427 | AW194270 | Hs.177236 | ESTs | | 2.7 |
| 450375 | AA009647 | Hs.8850 | a disintegrin and metalloproteinase domain | | 2.7 |
| 446999 | AA151520 | Hs 279525 | hypothetical protein PRO2605 | | 2.7 |
| 428819 | AL135623 | Hs.193914 | KIAA0575 gene product | | 2.7 |
| 422956 | BE545072 | Hs.122579 | ESTs | | 2.7 |
| 428949 | AA442153 | Hs 104744 | ESTs, Weakly similar to AF208855 1 BM-0 | | 2.7 |
| 426300 | U15979 | Hs.169228 | delta-like homolog (Drosophila) | TM, EGF | 2.6 |
| 420380 | AA640891 | Hs.102406 | ESTs | | 2.6 |
| 428651 | AF196478 | Hs.188401 | annexin A10 | TM, annexin | 2.6 |
| 417849 | AW291587 | Hs.82733 | Nidogen 2 | EGF, ldl_recept_b | 2.6 |
| 453700 | AB009426 | Hs.560 | apolipoprotein B mRNA editing enzyme, ca | TM | 2.6 |
| 417975 | AA641836 | Hs.30085 | Homo sapiens cDNA: FLJ23186 fis, clone | | 2.6 |
| 448756 | AI739241 | Hs.171480 | ESTs | | 2.6 |
| 425087 | R62424 | Hs.126059 | ESTs | | 2.5 |
| 444153 | AK001610 | Hs.10414 | hypothetical protein FLJ10748 | Kelch | 2.5 |
| 443211 | AI128388 | Hs.143655 | ESTs | | 2.5 |
| 415263 | AA948033 | Hs 130853 | ESTs | histone | 2.5 |
| 432867 | AW016936 | Hs.233364 | ESTs | GSHPx | 2.5 |
| 438639 | AI278360 | Hs.31409 | ESTs | | 2.5 |
| 455386 | AW935875 | | gb′QV3-DT0019-120100-055-d06DT0019 | | 2.5 |
| 419092 | J05581 | Hs.89603 | mucin 1, transmembrane | TM, SEA | 2.5 |
| 452055 | AI377431 | Hs.293772 | ESTs | | 2.5 |

Table 3 shows 92 genes up-regulated in mucinous-type ovarian cancer compared to normal adult tissues. These were selected as for TABLE 1, except that the “average” ovarian cancer level was set to the 75^{th} percentile amongst six mucinous-type ovarian cancers, and the tumor/normal tissue ratio was greater than or equal to 2.5.

TABLE 4
183 UP-REGULATED GENES, ENDOMETRIOID OVARIAN CANCER VERSUS NORMAL ADULT TISSUES

| | Exemplar | | | protein | ratio, |
|---|---|---|---|---|---|
| Primekey | Accession | UniGene | | structural | tumor |
| tissues | normal | ID | Title | domains | vs. |
| 452838 | U65011 | Hs.30743 | Preferentially expressed antigen in melanom | | 38.9 |
| 435094 | AI560129 | Hs 277523 | EST | | 28.8 |
| 428153 | AW513143 | Hs.98367 | hypothetical protein FLJ22252 similar to SR | | 24.1 |
| 428187 | AI687303 | Hs.285529 | ESTs | | 23.9 |
| 449034 | AI624049 | | gb:ts41a09.x1 NCI_CGAP_Ut1 Homo sapi | | 19.9 |
| 453102 | NM_007197 | Hs.31664 | frizzled (Drosophila) homolog 10 | TM, Fz, Frizzled | 15.7 |
| 412925 | AI089319 | Hs 179243 | ESTs | | 15.7 |
| 438817 | AI023799 | Hs.163242 | ESTs | | 13.6 |
| 447033 | AI357412 | Hs 157601 | EST - not in UniGene | PH | 13.5 |
| 433222 | AW514472 | Hs.238415 | ESTs, Moderately similar to ALU8_HUMA | | 13.1 |
| 422956 | BE545072 | Hs.122579 | ESTs | | 12.9 |
| 450451 | AW591528 | Hs 202072 | ESTs | | 11.9 |
| 453964 | AI961486 | Hs 12744 | ESTs | homeobox | 11.5 |
| 442438 | AA995998 | | gb:os26b03.s1 NCI_CGAP_KidS Homo sa | | 11.4 |
| 431989 | AW972870 | Hs 291069 | ESTs | SS | 10.3 |
| 413623 | AA825721 | Hs 246973 | ESTs | | 9.7 |
| 440901 | AA909358 | Hs 128612 | ESTs | | 9.6 |
| 416661 | AA634543 | Hs.79440 | IGF-II mRNA-binding protein 3 | KH-domain | 9.6 |
| 421478 | AI683243 | Hs.97258 | ESTs | | 9.3 |
| 448706 | AW291095 | Hs.21814 | class II cytokine receptor ZCYTOR7 | SS, Tissue_fac | 9.2 |
| 410566 | AA373210 | Hs 43047 | Homo sapiens cDNA FLJ13585 fis, clone P | | 8.7 |
| 438993 | AA828995 | | integrin; beta 8 | SS, integrin_B | 8.7 |
| 427121 | AI272815 | Hs 173656 | KIAA0941 protein | C2, | 8.4 |
| 420610 | AI683183 | Hs 99348 | distal-less homeo box 5 | homeobox | 8.1 |
| 427356 | AW023482 | Hs.97849 | ESTs | | 8.0 |
| 446577 | AB040933 | Hs 15420 | K1AA 1500 protein | | 8.0 |
| 431118 | BE264901 | Hs.250502 | carbonic anhydrase VIII | carb_anhydrase | 7.5 |
| 448112 | AW245919 | Hs 301018 | ESTs, Weakly similar to ALUB_HUMAN | | 6.9 |
| 451106 | BE382701 | Hs.25960 | N-myc | HLH, Myc_N_term | 6.6 |
| 449433 | AI672096 | Hs.9012 | ESTs | | 6.3 |
| 453922 | AF053306 | Hs.36708 | budding uninhibited by benzimidazoles 1 (y | | 6.3 |
| 434636 | AA083764 | Hs 241334 | ESTs | | 6.1 |
| 453688 | AW381270 | Hs.194110 | Homo sapiens mRNA; cDNA DKFZp434C | | 5.9 |
| 422805 | AA436989 | Hs 121017 | H2A histone family, member A | histone | 5.8 |
| 400292 | AA250737 | Hs 72472 | BMPR-Ib; bone morphogenetic protein rec | | 5.7 |
| 443179 | AI928402 | Hs.6933 | Homo sapiens cDNA FLJ12684 fis, clone N | | 5.6 |
| 418134 | AA397769 | Hs.86617 | ESTs | | 5.5 |
| 452249 | BE394412 | Hs 61252 | ESTs | homeobox | 5.5 |
| 409269 | AA576953 | Hs 22972 | Homo sapiens cDNA FLJ13352 fis, clone O | TM, UPF0016 | 5.5 |
| 413335 | AI613318 | Hs.48442 | ESTs | | 5.4 |
| 441081 | AI584019 | Hs 169006 | ESTs, Moderately similar to plakophilin 2b | PAX | 5.4 |
| 428029 | H05840 | Hs.293071 | ESTs | | 5.3 |
| 419183 | U60669 | Hs 89663 | cytochrome P450, subfamily XXIV (vitami | p450 | 5.3 |
| 409094 | AW337237 | | gb:xw82ffll x1 NCI_CGAP_Pan 1 Homo sa | | 5.2 |
| 432938 | T27013 | Hs.3132 | steroidogenic acute regulatory protein | START | 5.1 |
| 410102 | AW248508 | Hs 279727 | ESTs; | SS | 5.1 |
| 447835 | AW591623 | Hs.164129 | ESTs | | 5.1 |
| 438202 | AW1 69287 | Hs 22588 | ESTs | | 5.0 |
| 423992 | AW898292 | Hs 137206 | Homo sapiens mRNA; cDNA DKFZp564H | | 5.0 |
| 425905 | AB032959 | Hs.161700 | KIAA1133 protein | TM | 5.0 |
| 452461 | N78223 | Hs 108106 | transcription factor | G9a, PHD | 4.9 |
| 430691 | C14187 | Hs.103538 | ESTs | | 4.8 |
| 441675 | AI914329 | Hs.5461 | ESTs | | 4.7 |
| 425695 | NM_005401 | Hs.159238 | protein tyrosine phosphatase, non-receptor | Band_41, Y_phosphatase | 4.6 |
| 440340 | AW895503 | Hs.125276 | ESTs | | 4.5 |
| 428579 | NM_005756 | Hs.184942 | G protein-coupled receptor 64 | TM | 4.5 |
| 444783 | AK001468 | Hs 62180 | ESTs | PH | 4.4 |
| 451459 | AI797515 | Hs.270560 | ESTs, Moderately similar to ALU7 HUMA | | 4.4 |
| 413395 | AI266507 | Hs.145689 | ESTs | | 4.3 |
| 415263 | AA948033 | Hs 130853 | ESTs | histone | 4.2 |
| 413988 | M81883 | Hs 75668 | glutamate decarboxylase 1 (brain, 67 kD) | pyridoxal_deC | 4.2 |
| 452030 | AL137578 | Hs.27607 | Homo sapiens mRNA; cDNA DKFZp564N | | 4.1 |
| 418852 | BE537037 | Hs.273294 | hypothetical protein FLJ20069 | | 4.1 |
| 446431 | R45652 | Hs.153486 | ESTs | | 4.1 |
| 434891 | AA814309 | Hs 123583 | ESTs | | 4.0 |
| 415139 | AW975942 | Hs 48524 | ESTs | G-patch | 4.0 |
| 453197 | AI916269 | Hs.109057 | ESTs, Weakly similar to ALU5_HUMAN A | | 4.0 |
| 447112 | H17800 | Hs.7154 | ESTs | | 3.9 |
| 420633 | NM_014581 | Hs.99526 | odorant-binding protein 2B | TM.lipocalin | 3.9 |
| 459574 | AI741122 | Hs.101810 | Homo sapiens cDNA FLJ14232 fis, clone N | | 3.9 |
| 415138 | C18356 | Hs 78045 | tissue factor pathway inhibitor 2 TFPI2 | Kumtz_BPTI, G-gamma | 3.9 |
| 414083 | AL121282 | Hs 257786 | ESTs | | 3.7 |
| 442006 | AW975183 | Hs.292663 | ESTs | | 3.7 |
| 409731 | AA125985 | Hs 56145 | thymosin, beta, identified in neuroblastoma | Thymosin | 3.7 |
| 424906 | AI566086 | Hs 153716 | Homo sapiens mRNA for Hmob33 protein, | | 3.7 |
| 456662 | NM_002448 | Hs.1494 | msh (Drosophila) homeo box homolog 1 (fo | homeobox | 3.7 |
| 429125 | AA446854 | Hs.271004 | ESTs | | 3.6 |
| 435538 | AB011540 | Hs.4930 | low density lipoprotein receptor-related pro | | 3.6 |
| 458861 | AI630223 | | gb:ad06g08.r1 Proliferating Erythroid Cells | PHD | 3.5 |
| 418506 | AA084248 | Hs.85339 | G protein-coupled receptor 39 | | 3.5 |
| 423123 | NM_012247 | Hs.124027 | SELENOPHOSPHATE SYNTHETASE; H | AIRS.AIRS | 3.4 |
| 437960 | AI669586 | Hs.222194 | ESTs | | 3.4 |
| 400298 | AA032279 | Hs.61635 | STEAP1 | TM | 3.4 |
| 407162 | N63855 | Hs.142634 | zinc finger protein | | 3.4 |
| 408621 | AI970672 | Hs 46638 | chromosome 11 open reading frame 8; feta | | 3.3 |
| 445829 | AI452457 | Hs.145526 | ESTs | | 3.3 |
| 450262 | AW409872 | Hs 271166 | ESTs, Moderately similar to ALU7 HUMA | | 3.3 |
| 457979 | AA776655 | Hs.270942 | ESTs | TM | 3.3 |
| 402606 | #(NOCAT) | | | | 3.2 |
| 426471 | M22440 | Hs.170009 | transforming growth factor, alpha | SS.EGF | 3.2 |
| 430294 | AI538226 | Hs.135184 | ESTs | polyprenyl_synt | 3.2 |
| 448027 | AI458437 | Hs.177224 | ESTs | | 3.2 |
| 432619 | AW291722 | Hs 278526 | related to the N terminus of tre | TBC | 3.2 |
| 413627 | BE182082 | Hs 246973 | ESTs | | 3.2 |
| 441377 | BE218239 | Hs.202656 | ESTs | | 3.2 |
| 441085 | AW136551 | Hs.181245 | Homo sapiens cDNA FLJ12532 fis, clone N | | 3.2 |
| 433527 | AW235613 | Hs 133020 | ESTs | | 3.2 |
| 450171 | AL133661 | Hs.24583 | hypothetical protein DKFZp434C0328 | TM | 3.2 |
| 419807 | R77402 | | gb-yi75fl1.s1 Scares placenta Nb2HP Horn | | 3.1 |
| 418867 | D31771 | Hs 89404 | msh (Drosophila) homeo box homolog 2 | homeobox | 3.1 |
| 419335 | AW960146 | Hs.284137 | Homo sapiens cDNA FLJ12888 fis, clone N | | 3.1 |
| 450480 | X82125 | Hs 25040 | zinc finger protein 239 | zf-C2H2 | 3.1 |
| 420149 | AA255920 | Hs.88095 | ESTs | | 3.1 |
| 413415 | AA829282 | Hs 34969 | ESTs | | 3.1 |
| 438966 | AW979074 | | gb.EST391 184 MAGE resequences, MAGP | | 3.1 |
| 431041 | AA490967 | Hs.105276 | ESTs | Oxysterol_BP | 3.1 |
| 415245 | N59650 | Hs.27252 | ESTs | | 3.0 |
| 412140 | AA219691 | Hs.73625 | RAB6 interacting, kinesin-like (rabkmesm6 | kinesin | 3.0 |
| 431707 | R21326 | Hs 267905 | hypothetical protein FLJ10422 | | 3.0 |
| 448816 | AB033052 | Hs.22151 | KIAA1226 protein | | 3.0 |
| 447866 | AW444754 | Hs.21I5I7 | ESTs | homeobox | 3.0 |
| 450221 | AA328102 | Hs.24641 | cytoskeleton associated protein 2 | | 3.0 |
| 406997 | U07807 | Hs.194762 | Human metallothionein IV (MTIV) gene, c | | 3.0 |
| 433426 | H69125 | Hs.133525 | ESTs | TM | 3.0 |
| 420440 | NM_002407 | Hs.97644 | mammaglobin 2 | Uteroglobin | 3.0 |
| 420181 | AI380089 | Hs.158951 | ESTs | | 3.0 |
| 458627 | AW088642 | Hs 97984 | ESTs; Weakly similar to WASP-family pro | | 2.9 |
| 452055 | AI377431 | Hs.293772 | ESTs | | 2.9 |
| 429663 | M68874 | Hs 211587 | Human phosphatidylcholine 2-acylhydrolas | C2, PLA2_B | 2.9 |
| 415125 | AF061198 | Hs.301941 | Homo sapiens mRNA for norepmephrine tr | TM, SNF | 2.9 |
| 412708 | R26830 | Hs 106137 | ESTs | TM, 7tm_2, Rho_GDI | 2.9 |
| 451389 | N73222 | Hs21738 | KIAA1008 protein | | 2.9 |
| 423337 | NM_004655 | Hs 127337 | axin 2 (conductin, axil) | DIX.RGS | 2.9 |
| 435185 | AA669490 | Hs 289109 | dimethylarginine dimethylaminohydrolase | | 2.9 |
| 428054 | AI948688 | Hs.266619 | ESTs | | 2.9 |
| 448243 | AW369771 | Hs 77496 | ESTs | | 2.9 |
| 425723 | NM_014420 | Hs.159311 | dickkopf (Xenopus laevis) homolog 4 | SS | 2.9 |
| 432415 | T16971 | Hs 289014 | ESTs | | 2.9 |
| 414747 | U30872 | Hs.77204 | centromere protein F (350/400kD, mitosin) | | 2.9 |
| 400195 | | | 0 | | 2.9 |
| 449874 | AA 135688 | Hs.10083 | ESTs | | 2.8 |
| 452367 | U71207 | Hs.29279 | eyes absent (Drosophila) homolog 2 | Hydrolase | 2.8 |
| 428093 | AW594506 | Hs.104830 | ESTs | | 2.8 |
| 409640 | U78722 | Hs 55481 | zinc finger protein 165 | TM, zf-C2H2, SCAN | 2.8 |
| 424169 | AA336399 | Hs.153797 | ESTs | mito_carr | 2.8 |
| 409638 | AW450420 | Hs.21335 | ESTs | | 2.8 |
| 440048 | AA897461 | Hs 158469 | ESTs, Weakly similar to envelope protein [ | | 2.8 |
| 426890 | AA393167 | Hs 41294 | ESTs | | 2.8 |
| 452771 | T05477 | | gb:EST03366 Fetal brain, Stratagene (cat93 | | 2.8 |
| 422505 | AL120862 | Hs 124165 | ESTs; (HSA)PAP protein (programmed ce | | 2.8 |
| 416624 | H69044 | | gb.yr77h05 s1 Scares fetal liver spleen INF | zf-C3HC4 | 2.8 |
| 445870 | AW410053 | Hs 13406 | syntaxin 18 | TM | 2.7 |
| 441962 | AW972542 | Hs.289008 | Homo sapiens cDNA FLJ21814 fis, clone | | 2.7 |
| 447342 | AI199268 | Hs.19322 | ESTs; Weakly similar to !!!! ALU SUBFAM | | 2.7 |
| 421247 | BE391727 | Hs.102910 | general transcription factor IIH, polypeptid | | 2.7 |
| 419752 | AA249573 | Hs.152618 | ESTs | | 2.7 |
| 410658 | AW105231 | Hs 192035 | ESTs | | 2.7 |
| 437698 | R61837 | Hs.7990 | ESTs | | 2.7 |
| 458027 | L49054 | Hs 85195 | ESTs, Highly similar to t(3, 5)(q25.1; p34) f | | 2.7 |
| 438689 | AW129261 | Hs.250565 | ESTs | | 2.7 |
| 439876 | AI376278 | Hs.100921 | ESTs, Weakly similar to ALU7_HUMAN A | SCAN | 2.7 |
| 428479 | Y00272 | Hs 184572 | cell division cycle 2, G1 to S and G2 to M | pkinase | 2.7 |
| 436406 | AW105723 | Hs.125346 | ESTs | | 2.7 |
| 437938 | AI950087 | | ESTs, Weakly similar to Gag-Pol polyprote | | 2.7 |
| 419917 | AA320068 | Hs 93701 | Homo sapiens mRNA; cDNA DKFZp434E | | 2.7 |
| 434836 | AA651629 | Hs.118088 | ESTs | | 2.7 |
| 448404 | BE089973 | | gb:RC6-BT0709-310300-021-G07 BT0709 | | 2.7 |
| 444078 | BE246919 | Hs.10290 | U5 snRNP-specific 40 kDa protein (hPrp8- | WD40 | 2.7 |
| 409757 | NM_001898 | Hs.123114 | cystatin SN | SS.cystatin | 2.6 |
| 443775 | AF291664 | Hs.204732 | matrix metalloproteinase 26 | TM_, Peptidase_M10, 7tm_1 | 2.6 |
| 427961 | AW293165 | Hs 143134 | ESTs | | 2.6 |
| 426668 | AW136934 | Hs 97162 | ESTs | | 2.6 |
| 424717 | H03754 | Hs 152213 | wingless-type MMTV integration site fami | wnt | 2.6 |
| 434669 | AF151534 | Hs 92023 | core histone macroH2A2 2 | histone, Alpp, DUF27 | 2.6 |
| 417389 | BE260964 | Hs.82045 | Midkine (neurite growth-promoting factor 2 | SS, TM, PTN_MK | 2.6 |
| 451009 | AA013140 | Hs.115707 | ESTs | | 2.6 |
| 429774 | AI522215 | Hs.50883 | ESTs | pkinase | 2.6 |
| 439951 | AI347067 | Hs.124636 | ESTs | TM | 2.6 |
| 417576 | AA339449 | Hs 82285 | phosphoribosylglycinamide formyltransfera | AIRS, formyl_transf | 2.5 |
| 416806 | NM_000288 | Hs 79993 | peroxisomal biogenesis factor 7 | WD40 | 2.5 |
| 420900 | AL045633 | Hs.44269 | ESTs | Ald_Xan_dh_C | 2.5 |
| 457030 | AI301740 | Hs.173381 | dihydropyrimidinase-like 2 | Dihydroorotase | 2.5 |
| 459583 | AI907673 | | gb:IL-BT152-080399-004 BT152 Homo sa | | 2.5 |
| 440870 | AI687284 | Hs 150539 | Homo sapiens cDNA FLJ13793 fis, clone T | PAX, | 2.5 |
| 446693 | AW750373 | Hs.42315 | Homo sapiens cDNA FLJ13036 fis, clone N | TM | 2.5 |
| 407289 | AA135159 | Hs.203349 | Homo sapiens cDNA FLJ12149 fis, clone M | | 2.5 |
| 400882 | | | 0 | | 2.5 |
| 431322 | AW970622 | | gb.EST382704 MAGE resequences, MAGK | | 2.5 |
| 424081 | NM_006413 | Hs.139120 | ribonuclease P (30 kD) | | 2.5 |
| 451996 | AW514021 | Hs.245510 | ESTs | | 2.5 |
| 403381 | #(NOCAT) | | 0 | | 2.5 |
| 419488 | AA316241 | Hs.90691 | nucleophosmin/nucleoplasmin 3 | SS | 2.5 |
| 418882 | NM_004996 | Hs.89433 | ATP-binding cassette, sub-family C (CFTR | TM_, ABC_membrane | 2.5 |

Table 4 shows 183 genes up-regulated in endometrioid-type ovarian cancer compared to normal adult tissues. These were selected as for TABLE 1, except that the “average” ovarian cancer level was set to the 75^{th} percentile amongst seven endometrioid-type ovarian cancers, and the tumor/normal tissue ratio was greater than or equal to 2.5.

TABLE 5
178 UP-REGULATED GENES ENCODING SECRETED PROTEINS,
OVARIAN CANCER VERSUS NORMAL ADULT TISSUES

| | | | | ratio: |
|---|---|---|---|---|
| | | | | tumor |
| | | | | vs. |
| | Exemplar | UniGene | | normal |
| Primekey | Accession | ID | Title | tissues |
| 428579 | NM_005756 | Hs.184942 | G protein-coupled receptor 64 | 30.5 |
| 436982 | AB018305 | Hs.5378 | spondin 1, (f-spondin) extracellular mat | 29.4 |
| 427585 | D31152 | Hs.179729 | collagen; type X; alpha 1 (Schmid metaph | 27.0 |
| 423739 | AA398155 | Hs.97600 | ESTs | 22.7 |
| 418007 | M13509 | Hs.83169 | Matrix metalloprotease 1 (interstitial c | 20.6 |
| 438993 | M73780 | Hs.52620 | integrm; beta 8 | 16.7 |
| 428664 | AK001666 | Hs.189095 | similar to SALL1 (sal (Drosophila)-like | 16.5 |
| 439820 | AL360204 | Hs.283853 | Homo sapiens mRNA full length insert cDN | 16.5 |
| 400289 | X07820 | Hs.2258 | Matrix Metalloproteinase 10 (Stromolysin | 16.2 |
| 421155 | H87879 | Hs.102267 | lysyl oxidase | 16.1 |
| 431989 | AW972870 | Hs.291069 | ESTs | 15.9 |
| 426635 | BE395109 | Hs.129327 | ESTs | 15.9 |
| 424581 | M62062 | Hs.150917 | catenin (cadherin-associated protein), a | 15.7 |
| 428976 | AL037824 | Hs.194695 | ras homolog gene family, member I | 15.1 |
| 416209 | AA236776 | Hs.79078 | MAD2 (mitotic arrest deficient, yeast, h | 15.0 |
| 439706 | AW872527 | Hs.59761 | ESTs | 14.7 |
| 452055 | AI377431 | Hs.293772 | ESTs | 13.2 |
| 410102 | AW248508 | Hs.279727 | ESTs; | 12.5 |
| 428392 | H10233 | Hs.2265 | secretory granule, neuroendocrine protei | 12.4 |
| 402606 | AA434329 | Hs.36563 | hypothetical protein FLJ22418 | 11.5 |
| 443715 | A1583187 | Hs.9700 | cyclin El | 10.7 |
| 433496 | AF064254 | Hs.49765 | VLCS-H1 protein | 10.6 |
| 418601 | AA279490 | Hs.86368 | calmegin | 10.3 |
| 409269 | AA576953 | Hs.22972 | Homo sapiens cDNA FLJ13352 fis, | 10.1 |
| 445537 | AJ245671 | Hs.12844 | EGF-like-domain; multiple 6 | 9.9 |
| 427344 | NM000869 | Hs.2142 | 5-hydroxytryptamine (serotonin) receptor | 9.7 |
| 428479 | Y00272 | Hs.184572 | cell division cycle 2, G1 to S and G2 to | 9.7 |
| 429782 | NM005754 | Hs.220689 | Ras-GTPase-activating protein SH3-domain | 9.5 |
| 412140 | AA219691 | Hs.73625 | RAB6 interacting, kinesin-like (rabkines | 9.4 |
| 407881 | AW072003 | Hs.40968 | heparan sulfate (glucosamine) 3-O-sulfot | 9.4 |
| 435509 | AI458679 | Hs.181915 | ESTs | 9.3 |
| 408908 | BE296227 | Hs.48915 | serine/threonine kinase 15 | 9.0 |
| 433764 | AW753676 | Hs.39982 | ESTs | 9.0 |
| 445413 | AA151342 | Hs.12677 | CGI- 147 protein | 8.7 |
| 438078 | AI016377 | Hs.131693 | ESTs | 8.6 |
| 447342 | AI199268 | Hs.19322 | ESTs; Weakly similar to !!!! ALU SUBFA | 8.1 |
| 415138 | C18356 | Hs.78045 | tissue factor pathway inhibitor 2 TFPI2 | 7.7 |
| 418478 | U38945 | Hs.1174 | cyclin-dependent kinase inhibitor 2A (me | 7.5 |
| 426320 | W47595 | Hs.169300 | transforming growth factor, beta 2 | 7.5 |
| 424001 | W67883 | Hs.137476 | KIAA1051 protein | 7.4 |
| 458861 | NM007358 | Hs.31016 | DNA-BINDING PROTEIN M96 | 7.3 |
| 425465 | L18964 | Hs.1904 | protein kinase C; iota | 7.2 |
| 425776 | U25128 | Hs.159499 | parathyroid hormone receptor 2 | 7.1 |
| 424620 | AA101043 | Hs.151254 | kallikrein 7 (chymotryptic; stratum corn | 7.0 |
| 409178 | BE393948 | Hs.50915 | kallikrein 5 | 6.8 |
| 433159 | AB035898 | Hs.150587 | kinesin-like protein 2 | 6.6 |
| 410530 | M25809 | Hs.64173 | ESTs, Highly similar to VAB1 | 6.5 |
| 449048 | Z45051 | Hs.22920 | similar to S68401 (cattle) glucose induc | 6.5 |
| 422095 | A1868872 | Hs.288966 | ceruloplasmin (ferroxidase) | 6.4 |
| 425371 | D49441 | Hs.155981 | mesothelin | 6.4 |
| 448706 | AW291095 | Hs.21814 | class II cytokine receptor ZCYTOR7 | 6.4 |
| 441081 | AI584019 | Hs.169006 | ESTs, Moderately similar to plakophilin | 6.4 |
| 447207 | AA442233 | Hs.17731 | hypothetical protein FLJ 12892 | 6.3 |
| 420440 | NM_002407 | Hs.97644 | mammaglobin 2 | 6.2 |
| 457030 | AI301740 | Hs.173381 | dihydropyrimidinase-like 2 | 6.2 |
| 415139 | AW975942 | Hs.48524 | ESTs | 6.1 |
| 440870 | AI687284 | Hs.150539 | Homo sapiens cDNA FLJ 13793 fis, clone TH | 6.0 |
| 417866 | AW067903 | Hs.82772 | “collagen, type XI, alpha 1” | 6.0 |
| 437960 | AI669586 | Hs.222194 | ESTs | 6.0 |
| 410555 | U92649 | Hs.64311 | a disintegrin and metalloproteinase doma | 5.9 |
| 433447 | U29195 | Hs.3281 | neuronal pentraxin II | 5.9 |
| 437099 | N77793 | Hs.48659 | ESTs, Highly similar to LMA1 | 5.9 |
| 427510 | Z47542 | Hs.179312 | small nuclear RNA activating complex, po | 5.9 |
| 422867 | L32137 | Hs.1584 | cartilage oligomeric matrix protein | 5.8 |
| 444478 | W07318 | Hs.240 | M-phase phosphoprotein 1 | 5.7 |
| 445640 | AW969626 | Hs.31704 | ESTs, Weakly similar to K1AA0227 [H.sapi | 5.7 |
| 453775 | NM_002916 | Hs.35120 | replication factor C (activator 1) 4 (37 | 5.6 |
| 419917 | AA320068 | Hs.93701 | Homo sapiens mRNA; cDNA DKFZp434E232 | 5.6 |
| 424539 | L02911 | Hs.150402 | activin A receptor, type I | 5.5 |
| 441645 | AI222279 | Hs.201555 | ESTs | 5.5 |
| 424345 | AK001380 | Hs.145479 | Homo sapiens cDNA FLJ 105 18 fis, clone NT | 5.4 |
| 426514 | BE616633 | Hs.301122 | bone morphogenetic protein 7 (osteogenic | 5.4 |
| 425154 | NM 001851 | Hs.154850 | collagen, type IX, alpha 1 | 5.4 |
| 416530 | U62801 | Hs.79361 | kallikrein 6 (neurosin, zyme) | 5.3 |
| 445236 | AK001676 | Hs.12457 | hypothetical protein FLJ10814 | 5.2 |
| 452930 | AW195285 | Hs.194097 | ESTs | 5.2 |
| 431130 | NM_006103 | Hs.2719 | epididymis-specific; whey-acidic protein | 5.1 |
| 411571 | AA122393 | Hs.70811 | hypothetical protein FLJ20516 | 5.1 |
| 432158 | W33165 | Hs.55548 | ESTs, Weakly similar to unknown protein | 5.0 |
| 447020 | T27308 | Hs.16986 | hypothetical protein FLJ11046 | 5.0 |
| 443268 | AI800271 | Hs.129445 | hypothetical protein FLJ12496 | 4.9 |
| 448133 | AA723157 | Hs.73769 | folate receptor 1 (adult) | 4.9 |
| 418882 | NM_004996 | Hs.89433 | ATP-binding cassette, sub-family C (CFTR | 4.8 |
| 428555 | NM_002214 | Hs.184908 | integrin, beta 8 | 4.8 |
| 427528 | AU077143 | Hs.179565 | minichromosome maintenance deficient (S. | 4.7 |
| 406400 | AA343629 | Hs.104570 | kallikrein 8 (neuropsin/ovasin) | 4.7 |
| 439024 | R96696 | Hs 35598 | ESTs | 4.6 |
| 426300 | U15979 | Hs.169228 | delta-like homolog (Drosophila) | 4.6 |
| 448027 | AI458437 | Hs.177224 | ESTs | 4.6 |
| 404996 | NM_001333 | Hs.87417 | Cathepsin L2 | 4.6 |
| 443933 | AI091631 | Hs.135501 | ESTs | 4.5 |
| 409459 | D86407 | Hs.54481 | low density lipoprotein receptor-related | 4.4 |
| 414747 | U30872 | Hs.77204 | centromere protein F (350/400kD, mitosin | 4.3 |
| 423123 | NM_012247 | Hs.124027 | SELENOPHOSPHATE SYNTHETASE | 4.3 |
| 448275 | BE514434 | Hs.20830 | synaptic Ras GTPase activating protein 1 | 4.2 |
| 419926 | AW900992 | Hs.93796 | DKFZP586D2223 protein | 4.1 |
| 420736 | A1263022 | Hs.82204 | ESTs | 4.1 |
| 419790 | U79250 | Hs.93201 | glycerol-3-phosphate dehydrogenase 2 (mi | 4.1 |
| 414343 | AL036166 | Hs.75914 | coated vesicle membrane protein | 4.0 |
| 450654 | AJ245587 | Hs.25275 | Kruppel-type zinc finger protein | 4.0 |
| 445808 | AV655234 | Hs.298083 | ESTs | 3.9 |
| 417389 | BE260964 | Hs.82045 | Midkine (neurite growth-promoting factor | 3.9 |
| 425247 | NM_005940 | Hs.155324 | matrix metalloproteinase 11 (stromelysin | 3.8 |
| 430634 | AI860651 | Hs.26685 | ESTs | 3.8 |
| 431846 | BE019924 | Hs.271580 | Uroplakin 1B | 3.7 |
| 416658 | U03272 | Hs.79432 | fibrillin 2 (congenital contractural ara | 3.7 |
| 407792 | AI077715 | Hs.39384 | putative secreted ligand homologous to f | 3.7 |
| 420585 | AW505139 | Hs.279844 | hypothetical protein FLJ 10033 | 3.7 |
| 407756 | AA116021 | Hs.38260 | ubiquitin specific protease 1 8 | 3.6 |
| 411773 | NM_006799 | Hs.72026 | protease, serine, 21 (testisin) | 3.6 |
| 421928 | AF013758 | Hs.109643 | polyadenylate binding protein-interactin | 3.5 |
| 431958 | X63629 | Hs.2877 | Cadherin 3, P-cadherin (placental) | 3.5 |
| 410467 | AF102546 | Hs.63931 | dachshund (Drosophila) homolog | 3.5 |
| 418793 | AW382987 | Hs.88474 | prostaglandin-endoperoxide synthase 1 (p | 3.5 |
| 422278 | AF072873 | Hs.114218 | ESTs | 3.5 |
| 431840 | AA534908 | Hs.2860 | POU domain, class 5, transcription facto | 3.4 |
| 408730 | AV660717 | Hs.47144 | DKFZP586N0819 protein | 3.4 |
| 419452 | U33635 | Hs.90572 | PTK7 protein tyrosine kinase 7 | 3.3 |
| 421841 | AA908197 | Hs.108850 | KIAA0936 protein | 3.3 |
| 439864 | AI720078 | Hs.291997 | ESTs | 3.3 |
| 456546 | AI690321 | Hs.203845 | ESTs, Weakly similar to TWIK-related aci | 3.2 |
| 410687 | U24389 | Hs.65436 | lysyl oxidase-like 1 | 3.2 |
| 414774 | X02419 | Hs.77274 | plasminogen activator, urokinase | 3.2 |
| 420552 | AK000492 | Hs.98806 | hypothetical protein | 3.1 |
| 421991 | NM_014918 | Hs.110488 | KIAA0990 protein | 3.1 |
| 418140 | BE613836 | Hs.83551 | microfibrillar-associated protein 2 | 3.1 |
| 458924 | BE242158 | Hs.24427 | DKFZP5660 1646 protein | 3.1 |
| 411789 | AF245505 | Hs.72157 | Homo sapiens mRNA; cDNA DKFZp564I19 | 3.1 |
| 434241 | AF119913 | Hs.283607 | hypothetical protein PRO3077 | 3.1 |
| 422611 | AA158177 | Hs.118722 | fucosyltransferase 8 (alpha (1,6) fucosy | 3.1 |
| 409533 | AW969543 | Hs.21291 | mitogen-activated protein kinase kinase | 3.1 |
| 416391 | AI878927 | Hs.79284 | mesoderm specific transcript (mouse) hom | 3.1 |
| 412604 | AW978324 | Hs.47144 | DKFZP586N0819 protein | 3.1 |
| 425851 | NM_001490 | Hs.159642 | glucosaminyl (N-acetyl) transferase 1, c | 3.0 |
| 431259 | NM_006580 | Hs.251391 | claudin 16 | 3.0 |
| 418557 | BE140602 | Hs.246645 | ESTs | 3.0 |
| 428242 | H55709 | Hs.2250 | leukemia inhibitory factor (cholinergic | 3.0 |
| 419359 | AL043202 | Hs.90073 | chromosome segregation 1 (yeast homolog) | 3.0 |
| 457590 | AI612809 | Hs.5378 | spondin 1, (f-spondin) extracellular mat | 2.9 |
| 419741 | NM 007019 | Hs.93002 | ubiquitin carrier protein E2-C | 2.9 |
| 428330 | L22524 | Hs.2256 | matrix metalloproteinase 7 (matrilysin, | 2.9 |
| 417315 | AI080042 | Hs.180450 | ribosomal protein S24 | 2.9 |
| 438777 | AA825487 | Hs 142179 | ESTs, Weakly similar to ORF2 [M.musculus | 2.9 |
| 442295 | AI827248 | Hs.224398 | ESTs | 2.9 |
| 428248 | AI126772 | Hs.40479 | ESTs | 2.9 |
| 403019 | AA834626 | Hs.66718 | RAD54 (S.cerevisiac)-like | 2.8 |
| 436252 | AI539519 | Hs.120969 | Homo sapiens cDNA FLJ11562 fis | 2.8 |
| 419488 | AA316241 | Hs.90691 | nucleophosmin/nucleoplasmin 3 | 2.8 |
| 434288 | AW189075 | Hs.116265 | ESTs | 2.7 |
| 407872 | AB039723 | Hs.40735 | frizzled (Drosophila) homolog 3 | 2.7 |
| 431611 | U58766 | Hs.264428 | tissue specific transplantation antigen | 2.7 |
| 443881 | R64512 | Hs.237146 | Homo sapiens cDNA FLJ 14234 fis, clone NT | 2.7 |
| 453779 | N35187 | Hs.43388 | ESTs | 2.7 |
| 433068 | NM_006456 | Hs.288215 | sialyltransferase | 2.7 |
| 426841 | AI052358 | Hs.193726 | ESTs | 2.7 |
| 428778 | AK000530 | Hs.193326 | fibroblast growth factor receptor-like 1 | 2.7 |
| 451346 | NM_006338 | Hs.26312 | glioma amplified on chromosome 1 protein | 2.6 |
| 443883 | AA114212 | Hs.9930 | serine (or cysteine) protemase inhibito | 2.6 |
| 420162 | BE378432 | Hs.95577 | cyclin-dependent kinase 4 | 2.6 |
| 447149 | BE299857 | Hs.326 | TAR (HIV) RNA-binding protein 2 | 2.6 |
| 433656 | AW974941 | Hs.292385 | ESTs | 2.6 |
| 408210 | N81189 | Hs.43104 | ESTs | 2.6 |
| 430651 | AA961694 | Hs.105187 | kinesin protein 9 gene | 2.5 |
| 422599 | BE387202 | Hs.118638 | non-metastatic cells 1, protein (NM23A) | 2.5 |
| 421802 | BE261458 | Hs.108408 | CGI-78 protein | 2.5 |
| 446211 | A1021993 | Hs.14331 | SI 00 calcium-binding protein A13 | 2.5 |
| 404029 | W72881 | Hs.266470 | protocadherin beta 2 | 2.5 |
| 453012 | T95804 | Hs.31334 | putative mitochondrial outer membrane pr | 2.5 |
| 419981 | AA897581 | Hs.128773 | ESTs | 2.5 |
| 448153 | Y10805 | Hs.20521 | HMT1 (hnRNP methyltransferase, S. cerevi | 2.5 |
| 419220 | AA811938 | Hs.291759 | ESTs | 2.5 |
| 432180 | Y18418 | Hs.272822 | RuvB (Ecoli homolog)-like 1 | 2.4 |
| 406850 | AI624300 | Hs.172928 | collagen, type I, alpha 1 | 2.4 |
| 409893 | AW247090 | Hs.57101 | minichromosome maintenance deficient (S. | 2.4 |
| 421654 | AW163267 | Hs.106469 | suppressor of var1 (S.cerevisiae) 3-like | 2.4 |
| 409956 | AW103364 | Hs.727 | H. sapiens activin beta-A subunit (exon 2 | 2.4 |
| 407584 | W25945 | Hs.18745 | ESTs | 2.4 |
| 448796 | AA147829 | Hs.33193 | ESTs, Highly similar to AC007228 3 BC372 | 2.4 |
| Table 5 shows 178 genes up-regulated in ovarian cancer compared to normal adult tissues that are likely to encode proteins that are secreted into blood, lymph, or other bodily fluids. These genes, and/or their protein products, | | | | |
| # in combination or alone, are ideal candidates for the early diagnosis of ovarian cancer. These were selected from 59680 probesets on the Affymetrix/Eos Hu03 GeneChip array such that the ratio of “average” ovarian cancer to | | | | |
| # “average” normal adult tissues was greater than or equal to 2.4, and that are likely to encode secreted or extracellularly-shed proteins. The “average” ovarian cancer level was set to the 90^{th} percentile amongst | | | | |
| # 56 ovarian cancers obtained from the Garvan Institute for Molecular Research, Sydney, Australia. The “average” normal adult tissue level was set to the 90^{th} percentile amongst 149 non-malignant tissues. In order to remove | | | | |
| # gene-specific background levels of non-specific hybridization, the 15^{th} percentile value amongst the 149 non-malignant tissues was subtracted from both the numerator and the denominator before the ratio was evaluated. | | | | |

TABLE 6
17 GENES, AND COMBINATIONS THEREOF, USEFUL
FOR DIAGNOSIS OF OVARIAN CANCER

| | | |
|---|---|---|
| | | percent of tumors |
| UniGene ID | Title | detected (n = 56) |

Single genes:

| | | |
|---|---|---|
| Hs.5378 | spondin 1, (f-spondin) extracellular matrix protein | 77 |
| Hs.12844 | EGF-like-domain 6 | 86 |
| Hs.151254 | kallikrein 7 (chymotryptic; stratum corneum) | 66 |
| Hs.97644 | mammaglobin 2 | 73 |
| Hs.155981 | mesothelin (cytokine) | 57 |
| Hs.2258 | Matrix Metalloproteinase 10 (Stromolysin 2) | 21 |
| Hs.50915 | kallikrein 5 | 27 |
| Hs.301122 | bone morphogenetic protein 7 (osteogenic protein 1) (BMP7) | 54 |
| Hs.79361 | kallikrein 6 (neurosin, zyme) | 38 |
| Hs.83169 | MMP 1 (interstitial collagenase) | 23 |
| Hs.72026 | protease, serine, 2 1 (testisin) | 16 |
| Hs.39384 | putative secreted ligand homologous to fjx1 | 46 |
| Hs.2719 | epididymis-specific; whey-acidic protein type; four-disulfide core | 91 |
| Hs.155324 | matrix metalloproteinase 11 (stromelysin 3) | 11 |
| Hs.1584 | cartilage oligomeric matrix protein | 25 |
| Hs.169300 | TGF beta 2 | 21 |
| Hs.2250 | leukemia inhibitory factor (cholinergic differentiation factor) | 23 |

Exemplary Combinations:

| | EGF-like-domain 6 + mammaglobin 2 | 93 |
|---|---|---|
| | kallikrein 7 + mesothelin | 71 |
| | mammaglobin 2 + bone morphogenic protein 7 | 88 |
| | EGF-like-domain 6 + bone morphogenic protein 7 | 91 |
| | kallikrein 7 + bone morphogenic protein 7 + testisin | 75 |
| | kallikrein 7 + mammaglobin 2 + mesothelin | 84 |
| | mammaglobin 2 + bone morphogenic protein 7 + TGF beta 2 | 91 |
| | EGF-like-domain 6 + bone morphogenic protein 7 + MMP 1 | 95 |
| Table 6 shows 17 genes up-regulated in ovarian cancer compared to normal adult tissues that are likely to encode proteins that are secreted into blood, lymph, or other bodily fluids. These genes, and/or their protein products, in combination | | |
| # or alone, are ideal candidates for the early diagnosis of ovarian cancer. These were selected from 59680 probesets on the Affymetrix/Eos Hu03 GeneChip array such that the ratio of “average” ovarian cancer to “average” normal adult | | |
| # tissues was greater than or equal to 2.4, and that are likely to encode secreted or extracellularly-shed proteins. The “average” ovarian cancer level was set to the 90^{th} percentile amongst 56 ovarian cancers obtained from the Garvan | | |
| # Institute for Molecular Research, Sydney, Australia. The “average” normal adult tissue level was set to the 90^{th} percentile amongst 149 non-malignant tissues. In order to remove gene-specific background levels of non-specific hybridization, | | |
| # the 15^{th} percentile value amongst the 149 non-malignant tissues was subtracted from both the numerator and the denominator before the ratio was evaluated. | | |

It is understood that the examples described above in no way serve to limit the true scope of this invention, but rather are presented for illustrative purposes. All publications, sequences of accession numbers, and patent applications cited in this specification are herein incorporated by reference as if each individual publication or patent application were specifically and individually indicated to be incorporated by reference.

What is claimed is:

1. A method of detecting a ovarian cancer-associated transcript in a cell from a patient, the method comprising contacting a biological sample from the patient with a polynucleotide that selectively hybridizes to a sequence at least 80% identical to a sequence as shown in Tables 1-6.

2. The method of claim 1, wherein the biological sample comprises isolated nucleic acids.

3. The method of claim 2, wherein the nucleic acids are mRNA.

4. The method of claim 2, further comprising the step of amplifying nucleic acids before the step of contacting the biological sample with the polynucleotide.

5. The method of claim 1, wherein the polynucleotide comprises a sequence as shown in Tables 1-6.

6. The method of claim 1, wherein the polynucleotide is immobilized on a solid surface.

7. The method of claim 1, wherein the patient is undergoing a therapeutic regimen to treat ovarian cancer.

8. The method of claim 1, wherein the patient is suspected of having ovarian cancer.

9. An isolated nucleic acid molecule consisting of a polynucleotide sequence as shown in Tables 1-6.

10. The nucleic acid molecule of claim 9, which is labeled.

11. An expression vector comprising the nucleic acid of claim 9.

12. A host cell comprising the expression vector of claim 11.

13. An isolated polypeptide which is encoded by a nucleic acid molecule having polynucleotide sequence as shown in Tables 1-6.

14. An antibody that specifically binds a polypeptide of claim 13.

15. The antibody of claim 14, further conjugated to an effector component.

16. The antibody of claim 15, wherein the effector component is a fluorescent label.

17. The antibody of claim 15, wherein the effector component is a radioisotope or a cytotoxic chemical.

18. The antibody of claim 15, which is an antibody fragment.

19. The antibody of claim 15, which is a humanized antibody

20. A method of detecting a ovarian cancer cell in a biological sample from a patient, the method comprising contacting the biological sample with an antibody of claim 14.

21. The method of claim 20, wherein the antibody is further conjugated to an effector component.

22. The method of claim 21, wherein the effector component is a fluorescent label.

23. A method for identifying a compound that modulates a ovarian cancer-associated polypeptide, the method comprising the steps of:
(i) contacting the compound with a ovarian cancer-associated polypeptide, the polypeptide encoded by a polynucleotide that selectively hybridizes to a sequence at least 80% identical to a sequence as shown in Tables 1-6; and
(ii) determining the functional effect of the compound upon the polypeptide.

24. A drug screening assay comprising the steps of
(i) administering a test compound to a mammal having ovarian cancer or a cell isolated therefrom;
(ii) comparing the level of gene expression of a polynucleotide that selectively hybridizes to a sequence at least 80% identical to a sequence as shown in Tables 1-6 in a treated cell or mammal with the level of gene expression of the polynucleotide in a control cell or mammal, wherein a test compound that modulates the level of expression of the polynucleotide is a candidate for the treatment of ovarian cancer.
